(19) United States
(12) Patent Application Publication
(10) Pub. No.: US 2024/0277791 A1
(43) Pub. Date: Aug. 22, 2024

(54) DEVICE AND METHODS FOR ACNE THERAPEUTICS: ANTIBACTERIAL BACTERIOPHAGES AND ENGINEERED LYSINS

(71) Applicant: Integrated Pharma Services, LLC, Rockville, MD (US)

(72) Inventors: Mina Izadjoo, Rockville, MD (US); Hosan Kim, Ellicott City, MD (US); Salman Izadjoo, Rockville, MD (US)

(73) Assignee: Integrated Pharma Services, LLC, Rockville, MD (US)

(21) Appl. No.: 18/568,574

(22) PCT Filed: Jun. 10, 2022
(86) PCT No.: PCT/US22/33106

§ 371 Date: Dec. 8, 2023

Related U.S. Application Data
(60) Provisional application No. 63/209,469, filed on Jun. 11, 2021.

(51) Int. Cl. A61K 35/76; A61K 9/00; A61K 9/06; A61K 38/16; A61K 38/47; A61K 47/34; A61K 47/38; A61P 17/10; A61P 31/04; C07K 14/005; C12N 7/00; C12N 9/36

(52) U.S. Cl. CPC A61K 35/76; A61K 9/0014; A61K 9/06; A61K 38/162; A61K 38/47; A61K 47/34; A61K 47/38; A61P 17/10; A61P 31/04; C07K 14/005; C12N 7/00; C12N 9/2462; C12Y 302/01017; C12N 2795/00021; C12N 2795/00022; C12N 2795/00032

(57) ABSTRACT

Recombinantly-modified Cutibacterium acnes-specific bacteriophage, and recombinantly modified bacteriophage-derived endolysin enzyme are provided. Such compositions, alone or in combination with other antimicrobial agents or immunomodulators, may be used in pharmaceutical compositions, particularly pharmaceutical compositions to treat acne, promote wound healing, inhibit the growth of biofilms and to decrease the likelihood of, prevent or treat surgical related infections. The present invention is particularly directed to such formulations that are compounded for topical administration to a subject or used for application to medical devices. In particular, such pharmaceutical compositions may be compounded for topical administration to a subject, or compounded for application to a medical device.

### CROSS-REFERENCE TO RELATED APPLICATIONS

This Application is a national stage application under 35 U.S.C. 371 of International Patent Application No. PCT/US22/33106, which claims priority to U.S. Provisional Patent Appln. Ser. No. 63/209,469 (filed on Jun. 11, 2021), which applications are hereby incorporated by reference herein in their entirety.

### REFERENCE TO SEQUENCE LISTING

This application includes one or more Sequence Listings pursuant to 37 C.F.R. 1.821 et seq., which are disclosed in computer-readable media (file name: 1470_0002PCT_ST25.tx, created on Jun. 10, 2022, and having a size of 102,718 bytes), which file is herein incorporated by reference in its entirety.

### FIELD OF THE INVENTION

The present invention is directed to recombinantly-modified Cutibacterium acnes-specific bacteriophages, and to recombinantly modified variants of the bacteriophage-encoded endolysin enzyme. The present invention is further directed to pharmaceutical compositions that comprise therapeutically effective amounts of such compositions, alone, or more preferably, in combination with other antimicrobial agents or with immunomodulators, to treat acne, promote wound healing, inhibit the growth of biofilms and/or prevent (i.e., decrease the likelihood of) or treat surgical related infections. The present invention is particularly directed to pharmaceutical compositions that are compounded for topical administration to a subject or for application to a medical device.

### BACKGROUND OF THE INVENTION

### I. The Etiology of Acne

Acne is a widespread chronic disease on the face, neck and upper torso. Its pathogenesis due to increased sebum production, altered maturation and migration of keratinocytes, and inflammatory response and follicular inhabitation by the skin bacterium Cutibacterium acnes (C. acnes) formerly known as Propionibacterium acnes, which is Gram-positive and non-motile facultative anaerobic bacteria. These circumstances allow C. acnes to increase in numbers and trigger skin inflammation. This bacterial strain is known to elicit an inflammatory response and contribute to formation of acne lesions. This organism is also responsible for other infections including surgical procedures such as knee replacements. hip replacement, rotator cuff repairs and shoulder surgeries. C. acnes is responsible for more than 50% infections associated with such shoulder surgeries.

Acne vulgaris (or simply acne) is a common human skin disease, whose main effects, aside from causing scarring, are psychological, such as reduced self-esteem and in very extreme cases, depression or suicide. Acne is estimated to affect 9.4% of the global population, making it the eighth most prevalent disease worldwide. The global acne drugs market size is expected to reach USD 5.9 billion by 2025.

### II. Antibiotic Treatments for Acne

Antibiotics have been used to treat acne infections. Commonly topical antimicrobial chemicals, oral antibiotics or retinoids are applied for mild and moderate acnes. Both topical agents and oral antibiotics share serious side effects. Oral retinoids may be prescribed for more severe cases, but it could cause adverse effects such as dyslipidemia, altered blood glucose levels, eye and skin disorders and mood disorders. Furthermore, there has been an increase in emergence of antibiotic-resistant C. acnes strain mostly due to prolonged and overuse of antibiotics.

Commonly topical antimicrobial chemicals, oral antibiotics or retinoids are applied for mild and moderate acnes. Both topical agents and oral antibiotics share serious side effects. Moreover, the use of antibiotics to treat C. acnes infection is complicated by the increasing emergence of antibiotic resistant strains (Xu, H. et al. (2019) “Acne, the Skin Microbiome, and Antibiotic Treatment,” Am. J. Clin. Dermatol. 20(3):335-344). Non-antibiotic therapies for treating acne and surgical infections caused by C. acnes are urgently needed.

### III. Use of Bacteriophage in the Treatments of Acne

Bacteriophages (also known as “phages,”) are viruses that infect bacterial cells (Abril, A. G. et al. (2022) “The Use of Bacteriophages in Biotechnology and Recent Insights into Proteomics,” Antibiotics (Basel) 11(5):653:1-31; Venturini, C. et al. (2022) “Biological Foundations Of Successful Bacteriophage Therapy,” EMBO Mol Med. e12435:1-20; Aranaga, C. et al. (2022) “Phage Therapy in the Era of Multidrug Resistance in Bacteria: A Systematic Review,” Int. J. Mol. Sci. 23(9):4577:1-20; Azam, A. H. et al. (2021) “Bacteriophage Technology and Modern Medicine,” Antibiotics (Basel) 10(8):999:1-13). Bacteriophages attach to bacterial cells, and inject their viral genome into the cells. The infected bacterial cell expresses the encoded viral proteins, replicates the viral genome and ultimately kills the infected host cell, releasing additional bacteriophage particles that are then able to propagate the infection of additional bacterial cells.

Phages are often compared to antibiotics since both can be used as bacteriostatic or bacteriocidal agents. However, approximately three quarters of all bacterial infections are associated with bacteria that are embedded within self-produced extracellular matrices (“biofilms”) that antibiotics cannot typically penetrate (Visnapuu, A. et al. (2022) “Deconstructing the Phage-Bacterial Biofilm Interaction as a Basis to Establish New Antibiofilm Strategies,” Viruses 14(5):1057:1-19). Phages tend to be more successful than antibiotics in treating such infections (Chang, C. et al. “Bacteriophage-Mediated Control of Biofilm: A Promising New Dawn for the Future,” Front. Microbiol. 13:825828:1-14). Bacteriophages are much more specific than antibiotics. They are typically harmless not only to the host organism but also to other beneficial bacteria, such as the gut flora, thus reducing the chances of opportunistic infections. They have a high therapeutic index, that is, phage therapy would be expected to give rise to few side effects, even at higher-than therapeutic levels. Because phages replicate in vivo (in cells of a living organism), a smaller effective dose can be used.

Bacteriophage capable of infecting C. acnes have been described and proposed for use in the treatment of acne (Castillo, D. E. et al. (2019) “Propionibacterium (Cutibacterium) acnes Bacteriophage Therapy in Acne: Current Evidence and Future Perspectives,” Dermatol. Ther. (Heidelb). 9(1): 19-31; Jończyk-Matysiak, E. et al. (2017) “Prospects of Phage Application in the Treatment of Acne Caused by Propionibacterium acnes,” Front. Microbiol. 8:164:1-11; Brüggemann, H. et al. (2013) “Bacteriophages Infecting Propionibacterium acnes,” Biomed. Res. Int. 705741; Marinelli, L. J. et al. (2012) “Propionibacterium acnes Bacteriophages Display Limited Genetic Diversity And Broad Killing Activity Against Bacterial Skin Isolates,” mBio.3(5):e00279-12:1-13; Liu, J. et al. (2015) “The Diversity And Host Interactions Of Propionibacterium acnes Bacteriophages On Human Skin,” ISME J. 9(9):2078-2093; Meister, H. et al. (2022) “The Potential Role For Phage Therapy For Genetic Modification Of Cutaneous Diseases,” Clin. Dermatol. S0738-081X(22)00026-8:1-5; U.S. Pat. No. 9,068,159; US Patent Appln. Publn. No. 2021/0338569).

However, despite all such prior efforts, a need remains for pharmaceutical compositions capable of treating acne, promoting wound healing associated with C. acnes infection, inhibiting the growth of biofilms that comprise C. acnes, and/or preventing or treating surgical related C. acnes infections. The present invention is directed to these and other goals.

### SUMMARY OF THE INVENTION

The present invention is directed to recombinantly-modified Cutibacterium acnes-specific bacteriophage, and to recombinantly modified variants of the bacteriophage-encoded endolysin enzyme and holin proteins. The present invention is further directed to pharmaceutical compositions that comprise therapeutically effective amounts of such compositions, alone, or more preferably, in combination with other antimicrobial agents or with immunomodulators, to treat acne, promote wound healing, inhibit the growth of biofilms and/or prevent (i.e., decrease the likelihood of) or treat surgical related infections. The present invention is particularly directed to pharmaceutical compositions that are compounded for topical administration to a subject or for application to a medical device.

A recombinantly-modified Cutibacterium acnes-specific bacteriophage, and/or a recombinantly modified, bacteriophage-derived endolysin enzyme is provided. Such compositions, alone or in combination with other antimicrobial agents or immunomodulators, may be used in the pharmaceutical compositions of the present invention, particularly in pharmaceutical compositions to inhibit or kill acne-causing bacteria or to inhibit the growth of Cutibacterium acnes biofilms.

In detail, the invention provides a bacteriophage capable of inhibiting the growth of Cutibacterium acnes (C. acnes), wherein said bacteriophage comprises:
(A) bacteriophage φ50S, whose genome comprises the sequence of SEQ ID NO:1; or
(B) bacteriophage φ50M, whose genome comprises the sequence of SEQ ID NO:2;
or a variant bacteriophage capable of inhibiting the growth of C. acnes derived therefrom.

The invention further provides a bacteriophage endolysin capable of inhibiting the growth of C. acnes, wherein the endolysin comprises the amino acid sequence of SEQ ID NO:4 or SEQ ID NO:6, or is a variant endolysin derived therefrom.

The invention further provides a bacteriophage holin protein, wherein said holin protein comprises the amino acid sequence of SEQ ID NO:9, or is a variant holin protein derived therefrom.

The invention further provides a pharmaceutical composition that comprises a therapeutically effective amount of such bacteriophage, such bacteriophage endolysin, or such bacteriophage holin protein.

The invention further provides a pharmaceutical composition that comprises a prophylactically effective amount of such bacteriophage, such bacteriophage endolysin, or such bacteriophage holin protein.

The invention further provides a pharmaceutical composition that comprises prophylactically effective amounts or therapeutically effective amounts of such bacteriophage endolysin and of such bacteriophage holin protein.

The invention further provides the embodiment of such pharmaceutical compositions wherein the composition is a cream, gel, spray or liquid.

The invention further provides the embodiment of such pharmaceutical compositions wherein the composition comprises a hydrogel.

The invention further provides a bandage, medical device, or medical implant that comprises a prophylactically effective amount of any of such pharmaceutical compositions.

The invention further provides a bandage, medical device, or medical implant that comprises a therapeutically effective amount of any of such pharmaceutical compositions.

The invention further provides a method of treating a C. acnes infection, which comprises administering a therapeutically effective amount of any of such pharmaceutical compositions to a subject in need thereof.

The invention further provides the embodiment of such method wherein such pharmaceutical composition is administered topically to such subject.

The invention further provides the embodiment of such method wherein such C. acnes infection is a C. acnes biofilm infection.

The invention further provides a method of treating a C. acnes infection, which comprises administering a bandage, medical device, or medical implant that comprises a therapeutically effective amount of any of such pharmaceutical compositions to a subject in need thereof.

The invention further provides the embodiment of such method wherein such bandage, medical device, or medical implant is applied to a wound or surgical site of such subject, and particularly, wherein such wound or surgical site is a shoulder wound or shoulder surgical site.

The invention further provides a method of preventing (i.e., decreasing the likelihood of) a C. acnes infection, which comprises administering a prophylactically effective amount of any of such pharmaceutical compositions to a subject at risk of such infection.

The invention further provides the embodiment of such method wherein such pharmaceutical composition is administered topically to such subject.

The invention further provides the embodiment of such method wherein such pharmaceutical composition is administered as an aerosol.

The invention further provides a method of preventing (i.e. decreasing the likelihood of a C. acnes infection, which comprises administering a bandage, medical device, or medical implant that comprises a prophylactically effective amount of any of such pharmaceutical compositions to a subject at risk of such infection.

The invention further provides the embodiment of such method wherein such bandage, medical device, or medical implant is applied to a wound or surgical site of such subject.

The invention further provides the embodiment of such method wherein such wound or surgical site is a shoulder wound or shoulder surgical site.

The invention further provides a method of delivering therapeutic genes of the above-described bacteriophages, or of C. acnes, that comprises providing such above-described bacteriophages to subjects in need thereof. The invention thus further provides a use of C. acne bacteriophages phages to deliver therapeutic genes of such bacteriophages or of C. acnes to subjects in need thereof.

### BRIEF DESCRIPTION OF THE DRAWINGS

FIG. 1 shows the orientation and positioning of the PCR primers used to clone and modify the phiPA50S endolysin gene. In FIG. 1, a contiguous portion (SEQ ID NO:20) of SEQ ID NO:1 (nucleotides 15,297-16,766) is shown comprising the endolysin gene with a GTG start codon and a TGA stop codon. The start and stop codons of the polynucleotide encoding the endolysin gene are shown boxed and in uppercase letters. The start and stop codons of the polynucleotide encoding the holin gene are shown in uppercase letters.

FIG. 2 shows the orientation and positioning of the PCR primers Primer UniF, 50S_1 F, 50S_2 F and Primer 50S_3R used to clone and modify the phiPA50S endolysin gene. In FIG. 2, a contiguous portion (SEQ ID NO:20) of SEQ ID NO:1 (nucleotides 15,297-16,766) is shown which comprises the endolysin gene with an GTG start codon and a TGA stop codon and the holin gene with a GTG start codon and a TAA stop codon. The start and stop codons of the polynucleotide encoding the endolysin gene are shown boxed and in uppercase letters. The start and stop codons of the polynucleotide encoding the holin gene are shown in uppercase letters.

FIG. 3 shows the orientation and positioning of the PCR primers Primer 50S_2 F-NcA and Primer 50S_3R-St used to clone and modify the phiPA50S endolysin gene into the pBAD-TOTO TA vector. In FIG. 3, a contiguous portion (SEQ ID NO:21) of the genome of a recombinantly-produced phiPA50S variant is shown which comprises nucleotides 15,297-16,766 of SEQ ID NO:1, modified so that the endolysin gene starts with an ATG start codon and a GGA stop codon. The sequence includes the holin gene with its native GTG start codon and TAA stop codon. The start and stop codons of the polynucleotide encoding the endolysin gene are shown boxed and in uppercase letters. The start and stop codons of the polynucleotide encoding the holin gene are shown in uppercase letters.

FIG. 4 shows the orientation and positioning of the PCR primers Primer 50S_2 F-NcB and Primer 50S_3R-St, Primer 50S_1R or Primer 50S_2R used to clone and modify the phiPA50S endolysin gene into the pBAD-TOTO TA vector. In FIG. 4, a contiguous portion (SEQ ID NO:21) of the genome of a recombinantly-produced phiPA50S variant is shown which comprises nucleotides 15,297-16,766 of SEQ ID NO:1, modified so that the endolysin gene starts with an ATG start codon and a GGA stop codon. The sequence includes the holin gene with its native GTG start codon and TAA stop codon. The start and stop codons of the polynucleotide encoding the endolysin gene are shown boxed and in uppercase letters. The start and stop codons of the polynucleotide encoding the holin gene are shown in uppercase letters.

FIG. 5 shows the orientation and positioning of the PCR primers used to clone and modify the phiPA50S holin gene. In FIG. 5, a contiguous portion (SEQ ID NO:29) of SEQ ID NO:1 (nucleotides 16,137-16,766) is shown which comprises a portion of the endolysin gene and the holin gene with its native GTG start codon and TAA stop codon. The start and stop codons of the polynucleotide encoding the holin gene are shown boxed and in uppercase letters.

FIG. 6 shows the orientation and positioning of PCR primers used to subclone and modify the phiPA50S holin gene. In FIG. 6, a contiguous portion (SEQ ID NO:29) of SEQ ID NO:1 (nucleotides 16,137-16,766) is shown which comprises a portion of the endolysin gene and the holin gene with a GTG start codon and a TAA stop codon. The start and stop codons of the polynucleotide encoding the holin gene are shown boxed and in uppercase letters.

FIG. 7 shows the orientation and positioning of PCR primers used to subclone and modify the phiPA50S holin gene. In FIG. 7, a contiguous portion (SEQ ID NO:30) of the genome of a recombinantly-produced phiPA50S variant is shown which comprises nucleotides 16,137-16,766 of SEQ ID NO:1, modified so that the holin gene starts with an ATG start codon and terminates with a GAA stop codon. The start and stop codons of the polynucleotide encoding the holin gene are shown boxed and in uppercase letters.

FIG. 8 shows the results of a spot assay of 9 bacteriophages against C. acnes 6919 strain. 20 μl of a preparation of each phage was spotted in triplicate onto RCM (reinforced clostridium medium) agar plates having plated C. acnes 6901 cultures (approximately 10⁸ CFU/ml). About 2 inches of zone of inhibition was observed for 7 bacteriophages (Ø044, Ø86, Ø11828, Ø59, Ø50M, Ø50S, Ø86L) against C. acnes 6919 strains. Three phages (Ø86L, Ø86S, and Ø50L) showed different zone of inhibition.

FIG. 9 shows the results of a Top Agar Overlay Assay of 3 Bacteriophages against host strain C. acnes ATCC 6919. 300 μl of each phage was mixed with C. acnes ATCC 6901 cultures (150 μl of approximately 10⁸ CFU/ml) and poured onto RCA plates. C. acnes ATCC 6919 cultures (MOI=2) were mostly eradicated by Ø50L & Ø044 and were completely eradicated by Ø86.

FIGS. 10A and 10B show the expression of the endolysin of C. acnes-specific phage. The potential E. coli transformants including the full endolysin gene, LPA50S was expressed and verified with SDS-PAGE (FIG. 10A) and western blot (FIG. 10B) analysis. Two E. coli clones (PLys41 and PLys54) including (′. acnes-specific phage endolysin were successfully expressed and detected by western blot analysis with 6× His Tag epitope antibody. Marker: Pre-stained Protein Markers, E. coli (−): E. coli host strain as negative control, LacZ (+): LacZ gene expressed as positive control, PLys35: Cloned endolysin, PLys41: Cloned endolysin, PLys54: Cloned endolysin.

FIGS. 11A and 11B show the preparation of a semi-solid cream formulation of the pharmaceutical compositions of the present invention (FIG. 11A) and the lytic capacity of such formulation (FIG. 11B). Two C. acnes-specific bacteriophages (phiPA50M and phiPA50S) were selected to make cream formulations. One gram of Cetomacrogol cream (non-ionic) on a sterile glass slab (FIG. 11A, 1) was mixed with each phage preparation by gradual serial addition using a sterile stainless-steel blade (FIG. 11A, 2) to the cream to make a final concentration of 1.2 ×10⁸ PFU per gram for phiPA50M and 3.0×10⁶ PFU per gram for phiPA50S until all the fresh cream has been incorporated, and the phage were evenly dispersed throughout the cream. The phage cream was added to the 3 ml syringe wrapped the end with a parafilm for efficacy testing (FIG. 11A, 3). In order to assess lytic capacity of the C. acnes phage cream formulations, first (′. acnes ATCC 6919 culture was plated on RCM (reinforced clostridial medium) agar plate, secondly, each of the phage creams applied onto the surface, thirdly, the plates were incubated at 37ºC for 2 days under anaerobic condition to observe any lysis of the bacteria in the presence of the cream by a clear zone (FIG. 11B). Both phage phiPA50M and phiPA50S cream formulations showed strong activity against (′. acnes ATCC 6919.

FIG. 12 shows three (′. acnes phage formulation and their lytic capacity. Three types of C. acnes phage formulations using base materials such as hydroxyethylcellulose (HEC, nonionic gelling agent, 1.5% w/v), cetomacrogol cream (a non-ionic base), and poloxamer 407 (16% w/v) were prepared with (′. acnes-specific bacteriophage (phiPA50M). The (′. acnes phage formulations (approximately 1.0×10⁸ PFU per gram) were evaluated for their lytic capacity against (′. acnes ATCC 6919. Each of the phage formulations as marked with a black circle or white cream was applied onto the surface. The plates were incubated at 37ºC for 2 days under anaerobic condition to observe any lysis of the bacteria in the presence of the formulation by a clear zone. All phiPA50M phage formulations showed strong activity against C. acnes 6919.

### DETAILED DESCRIPTION OF THE INVENTION

The present invention is directed to recombinantly-modified Cutibacterium acnes-specific bacteriophage, and to recombinantly modified variants of the bacteriophage-encoded endolysin enzyme. The present invention is further directed to pharmaceutical compositions that comprise therapeutically effective amounts of such compositions, alone, or more preferably, in combination with other antimicrobial agents or with immunomodulators, to treat acne, promote wound healing, inhibit the growth of biofilms and/or prevent (i.e., decrease the likelihood of) or treat surgical related infections. The present invention is particularly directed to pharmaceutical compositions that are compounded for topical administration to a subject or for application to a medical device. The bacteriophages of the present invention can also be used as a diagnostic tool for detecting the presence of C. acnes, for screening for C. acnes and for surveilling a C. acnes infection.

As used herein, the term “acne” denotes a skin condition that occurs when hair follicles become plugged with oil and dead skin cells causing whiteheads, blackheads or pimples. It is most common among teenagers, but it affects people of all ages.

As used herein, the term “anaerobic” denotes in biology relatedness or requirement of an absence of free oxygen.

As used herein, the term “antibacterial” denotes a substance with prevention or inhibition of the growth or spread of bacteria.

As used herein, the term “antimicrobial agent” denotes a natural or synthetic substance that kills or inhibits or limit the growth of microorganisms such as bacteria, fungi, and algae.

As used herein, the term “bacteriophage” denotes a virus, which also called phage or bacterial virus, that infects and replicates within bacteria and archaea.

As used herein, the term “biofilm” denotes a complex structure of microbiome having different microbial colonies or single type of cells in a group.

As used herein, the term “chronic disease” denotes a disease or condition that usually lasts for more than 3 months and may get worse over time. It can usually be controlled but not cured.

As used herein, the term “clinical” denotes the observation and treatment of actual patients rather than theoretical or laboratory studies.

As used herein, the term “Cutibacterium acnes” denotes formerly Propionibacterium acnes, typically aerotolerant anaerobic gram-positive bacterium causing the skin condition of acne.

As used herein, the term “endolysin” denotes hydrolytic or peptidoglycan-degrading enzymes produced by bacteriophages to cleave bacterial host's cell wall. It is also known as lysin or murein hydrolase.

As used herein, the term “formulation” denotes a term used in various applications for making a material or mixture prepared in appropriate relationships or structures, according to a particular formula.

As used herein, the term “immunomodulator” denotes substances that can help support immune response or function by modifying, generally in a beneficial way.

As used herein, the term ‘inflammation’ denotes a localized physical condition in which part of the body becomes reddened, swollen, hot, and often painful, especially as a reaction to injury or infection.

As used herein, the term “lytic activity” denotes destruction of the infected cell and its membrane by bacterial viruses or bacteriophages.

As used herein, the term “optical density” denotes absorbance of a material that is a logarithmic intensity ratio of the light falling upon the material, to the light transmitted through the material.

As used in herein, the term “plaque” denotes a clear area in a bacterial culture produced by viral destruction of cells.

As used in herein, the term “preventing infection,” denotes reducing or eliminating the likelihood of infection of a subject at risk of such an infection.

As used herein, the term “propagation” denotes the action of widely spreading and amplifying viruses in biology.

As used herein, the term “recombinantly-modified” denotes [e.g., a variant or derivative of a natural isolate or enzyme that was created using recombinant DNA technology and that differs in amino acid or nucleotide sequence from such natural isolate.]

As used herein, the term “antibiotic resistant” denotes the ability of a bacterium to resist the bacteriocidal or bacteriostatic activity of an antimicrobial agent.

As used herein, the term “specificity” denotes in biology the narrowness of the range of substances with which an antibiotic or other agent acts or is effective. In medicine denotes the extent to which a diagnostic test is specific for a particular condition, trait, etc.

As used herein, the term “surgical site” denotes the part of a subject (e.g., an arm, leg, shoulder, head, chest, abdomen, etc.) that has been subjected to surgery. As used herein, the term “surgical site infection” denotes an infection that occurs, or that may occur, after surgery at a surgical site.

As used herein, the term “therapeutic” denotes the branch of medicine concerned with the treatment of disease and the action of remedial agents.

As used herein, the term “titer” denotes the strength of a solution or the concentration of a substance in solution as determined by titration.

As used herein, the term “top agar overlay” denotes that the cooling, liquid agar (usually less than 0.7%) is then poured evenly over a surface of a solid agar plate. It is a technique to evaluate surface and subsurface growth of a culture or create a lawn of microorganism for viral plaque assays or in some antibiotic (antimicrobial) sensitivity assays.

As used herein, the term “virion” denotes a complete virus particle that consists of an RNA or DNA core with a protein coat sometimes with external envelopes and that is the extracellular infectious form of a virus.

As used herein, the term “zone of inhibition” denotes a test, also called a Kirby-Bauer Test, which is a qualitative method used clinically to measure antibiotic or antimicrobial resistance and in industry to evaluate the ability of solids and textiles to inhibit microbial growth.

Aspects disclosed herein include methods and materials that are available to make various types of topical formulations to control or prevent acnes-causing bacteria and underlying biofilms on the skin. In addition, the formulation/s can be used to prevent surgical infections. The invention particularly contemplates the use of pharmaceutical compositions in which the C. acnes-specific bacteriophage, or its endolysin is encapsulated. “Encapsulation” represents the action of enclosing something in or as if in capsule, or the like. Encapsulation is designed for an efficient and stable delivery to deep skin or target area of a human body, and for protection of activity or efficacy of the active ingredients. Encapsulation as used herein encloses either phage(s), or an endolysin, or a mixture of phages and an endolysin or other antimicrobial agents with other enclosing materials such liposome, noisome or other vesicles. Liposome-encapsulated phage is available for biocontrol of C. acnes in skins and biofilms (Singla, S. et al. (2016) “Encapsulation Of Bacteriophage In Liposome Accentuates Its Entry Into Macrophage And Shields It From Neutralizing Antibodies,” PLOS One 11(4):e0153777:1-16).

Aspects disclosed herein include application to skin, medical implants (as a rinse or coating), pre- and post-surgical sites.

The invention also particularly contemplates the use of pharmaceutical compositions that comprise the use of hydrogels that comprise the (′. acnes-specific bacteriophage, or its endolysin is encapsulated. As used herein, the term “hydrogel” as used herein can be any type of a hydrogel, such as but not limited to gelatin, hydroxyethylylcellulose, hydroxypropylcellulose, methylcellulose, poloxamer, dextran, alginate, chitosan, collagen, dextran sulfate, PEG-PLA-PEG, poly(vinyl alcohol, PVA), poly(dimethylaminoethyl methacrylate, PDMAEMA) and polymer based. Hydrogel represents a three-dimensional network of hydrophilic polymers that can swell in water and hold a large amount of water while maintain the structure due to chemical or physical cross-linking of individual polymer chains. A preferred hydrogel is hydroxyethylcellulose (HEC) (Yang, K. et al. (218) “Antimicrobial Hydrogels: Promising Materials For Medical Application,” Int. J. Nanomedicine 13:2217-2263). Suitable compositions may comprise hydroxyethylcellulose (HEC, 1 g of 1.5%) or poloxamer 407 (1 g of 16%) and a C. acnes-specific phage (approximately 10⁹ PFU/ml).

Gelatin represents a translucent, colorless, flavorless food ingredient, derived from collagen taken from animal body parts. It is brittle when dry and gummy when moist. It may also be referred to as hydrolyzed collagen, collagen hydrolysate, gelatin hydrolysate, hydrolyzed gelatin, and collagen peptides after it has undergone hydrolysis. It is commonly used as a gelling agent in food, medications, drug and vitamin capsules, photographic films and papers, and cosmetics.

Hydroxypropyl cellulose (HPC) represents a derivative of cellulose with both water solubility and organic solubility. It is used as an excipient, and topical ophthalmic protectant and lubricant. HPC is an ether of cellulose in which some of the hydroxyl groups in the repeating glucose units have been hydroxypropylated forming —OCH₂CH(OH)CH₃ groups using propylene oxide. Because cellulose is very crystalline, HPC must have an MS about 4 in order to reach a good solubility in water. HPC has a combination of hydrophobic and hydrophilic groups, so it has a lower critical solution temperature (LCST) at 45° C. At temperatures below the LCST, HPC is readily soluble in water; above the LCST, HPC is not soluble.

Methyl cellulose (or methylcellulose) is a chemical compound derived from cellulose. Methyl cellulose is used in the manufacture of drug capsules; it is edible and its nontoxic properties provide a vegetarian alternative to the use of gelatin. Methyl cellulose is very occasionally added to hair shampoos, toothpastes and liquid soaps, to generate their characteristic thick consistency. This is also done for foods, for example ice cream or croquette. Methyl cellulose is also an important emulsifier, preventing the separation of two mixed liquids because it is an emulsion stabilizer. Methyl cellulose, as a gel, has the unique property of setting when hot and melting when cold. Methyl cellulose has a lower critical solution temperature (LCST) between 40° C. and 50° C. At temperatures below the LCST, it is readily soluble in water; above the LCST, it is not soluble, which has a paradoxical effect that heating a saturated solution of methyl cellulose will turn it solid, because methyl cellulose will precipitate out.

Hydrogel is preferably used with poloxamer 407 as a synthetic polymer. Poloxamer 407 (BASF) represents a hydrophilic non-ionic surfactant of the more general class of copolymers known as poloxamers. Poloxamer 407 is a triblock copolymer consisting of a central hydrophobic block of polypropylene glycol flanked by two hydrophilic blocks of polyethylene glycol (PEG). The approximate lengths of the two PEG blocks is 101 repeat units while the approximate length of the propylene glycol block is 56 repeat units. Poloxamer 407 represents a synthetic polymer that a safety assessment published in the International Journal of Toxicology found no evidence to suggest it is unsafe for use in cosmetic products. Poloxamer 407 are related to its surfactant properties in most of the common uses. For example, it is widely used in cosmetics for dissolving oily ingredients in water. It can also be found in multi-purpose contact lens cleaning solutions, where its purpose there is to help remove lipid films from the lens. It can also be found in some mouthwashes. Poloxamer 407 in a 30% solution by weight forms a gel solid at room temperature but liquifies when chilled to 4ºC. This allows poloxamer 407 to serve as a removable support material, particularly for creating hollow channels or cavities inside hydrogels.

Each type of hydrogels based on hydroxyethylcellulose (HEC, 1 g of 1.5%) or poloxamer 407 (1 g of 16%) was prepared and mixed with (′. acnes-specific phages phiPA50M (approximately 10⁹ PFU/ml) or phiPA50S (approximately 10⁹ PFU/ml).

The formulation can also be used with other components such as Crystal Violet and/or Methylene Blue.

I. Exemplary Therapeutic Compositions Comprising the C. acnes-Specific Bacteriophages φ50S and φ50M and/or their Endolysin

Aspects disclosed herein include novel methods, enzyme and formulations for isolating acne bacteriophages, for obtaining phage-derived endolysin enzyme, and for generating various types of bacteriophage-derived and lysin-derived topical formulations (e.g., creams, gels, sprays, aerosols, mists, liquids, encapsulated formulations, etc.). These formulated products inhibit or kill acne-causing bacteria, particularly C. acnes on face, neck, shoulder, back, and other body parts and surgical sites and medical implants, or may be used as fumigants (e.g., aerosolized fumigants or misted fumigants to inhibit or kill acne-causing bacteria in a space, such as a room (e.g., a hospital room, operating room, etc.), or in a wound. These formulated products may be combined with antimicrobial silver nanoparticles, copper nanoparticles, zinc nanoparticles, chitosan, graphene, peptides, lysozyme, CBD, an antimicrobial agent, or an immunomodulator, to increase antibacterial efficacy against C. acnes and/or to inhibit C. acnes causing infections.

In a first preferred embodiment, the compositions of the present invention will comprise a prophylactically effective amount or a therapeutically effective amount of the (′. acnes-specific bacteriophage φ50S (also referred to as “PhiPA50S”) and/or the C. acnes-specific bacteriophage φ50M (also referred to as “PhiPA50M”). Bacteriophage φ50S is a double-stranded DNA virus, whose 29,502 nucleotides comprise the sequence of SEQ ID NO:1. A initial genomic sequence of the (′. acnes phage φ50S was annotated and deposited in the GenBank database (Accession Number MT647607), which is herein incorporated by reference in its entirety. Bacteriophage φ50M is a double-stranded DNA virus, whose 29,491 nucleotides comprise the sequence of SEQ ID NO:2. The sequence of bacteriophage φ50M is similar to that of bacteriophage φ50S.

C. acnes-specific bacteriophage φ50S and C. acnes-specific bacteriophage φ50M encode identical endolysins. In a second preferred embodiment, the compositions of the present invention will comprise a prophylactically effective amount or a therapeutically effective amount of the endolysin encoded by C. acnes-specific bacteriophage φ50S or C. acnes-specific bacteriophage φ50M.

C. acnes, and other bacterial cells, may be induced to produce bacteriophage φ50M or φ50S by delivering the DNA of such bacteriophage (e.g., SEQ ID NO:1 or SEQ ID NO:2) into such cells using any of a variety of methods, such as electroporation, microprecipitation, microinjection, liposomal transfection, particle bombardment, sonoporation, laser-induced poration, bead transfection, magnetofection, etc. (Neshat, S. Y. et al. (2020) “Gene Delivery For Immunoengineering,” Curr. Opin. Biotechnol. 66:1-10; Lagarce, F. et al. (2016) “Nucleic-Acid Delivery Using Lipid Nanocapsules,” Curr. Pharm. Biotechnol. 17(8): 723-727; Miller, D. L. et al. (2002) “Sonoporation: Mechanical DNA Delivery By Ultrasonic Cavitation,” Somat. Cell Mol. Genet. 27(1-6): 115-134). Electroporation is a preferred method for delivering bacteriophage φ50M or φ50S genomic DNA into cells to produce infectious bacteriophage φ50M or φ50S particles. Methods for performing electroporation are described by Cheong, D. E. et al. (2007) “Optimization Of Electrotransformation Conditions For Propionibacterium Acnes,” J. Microbiol. Methods 72(1):38-41, by Janež, N. et al. (2019) “Introduction of Phage Genome into Escherichia coli by Electroporation,” Methods Mol. Biol. 1898:51-56:1-6, and by Drury, L. (1994) “Transformation of Bacteria by Electroporation,” In: METHODS ON MOLECULAR BIOLOGY, Humana Press (Harwood, A.J., Ed.), Volume 31, Chapter 1, Pages 1-8.

The endolysin of C. acnes-specific bacteriophage φ50S may be encoded starting from the start codon at position 15,414 of SEQ ID NO:1, to the stop codon which begins at position 16,269 of SEQ ID NO:1 (SEQ ID NO:3):

dtgagataca ttccagcggc gcatcactcg gccggatcaa ataagccggt
gaaccgtgtt gtgattcacg cgacatgccc ggatgtgggg tttccgtccg
cttcccgtaa ggggcgggcg gtgtctacag caaactattt tgcttcccca
tcgtcgggtg gttctgccca ttatgtgtgt gatgttgggg agacggtgca
gtgcttgtcg gagtctacga ttggttggca tgccccgccg aatccgcatt
ctttgggtat agagatttgc gcggatgggg gttcgcacgc ctcgttccgt
gtgccagggc atgcttacac gagggagcag tggctggatc ctcgggtgtg
gcccgccgtg gagcgtgccg ccatcctgtg tcggcagttg tgtgacaagc
atggtgttcc gaaaaggaag cttagcgtat ccgatttgaa ggccggtaaa
cgtggtgttt gcgggcatgc ggatgttacg gatgcgtggc atcagtcgga
tcatgacgat ccggggccgt ggtttccgtg ggacaggttt atggccgtag
tctgcggcgg tagtggagag agtgaggagt taactgtggc tgatgtgaaa
gccttgcatg atcagattaa acaattgtct gctcagctta gtggttcggt
gaataagctg caccatgatg ttggtgtggt tcaggtacag aatggtgatt
tgggtaagcg tgttgacgcc ctgtcgtggg tgaagaatcc ggtgacgggg
aagctgtggc gcaccaagga tgctttgtgg agtgtctggt attacgtgtt
ggagtgtcgt agccgtcttg acaggcttga gtctgctgtt aacggtttga
aaaagtrr

wherein: d at position 1 is either the native g, or may be a or t; and trr at positions 856-858 is either the native tga, or may be taa or tag.

More specifically, the endolysin of C. acnes-specific bacteriophage φ50S may be encoded by the embodiment of SEQ ID NO:3 in which the start codon for the encoded endolysin (nucleotide residues 1-3 of SEQ ID NO:3) is ttg, and the termination codon for the encoded endolysin (nucleotide residues 856-858 of SEQ ID NO:3) is tga, taa or tag. Alternatively, the endolysin of C. acnes-specific bacteriophage φ50S may be encoded by the embodiment of SEQ ID NO:3 in which the start codon (nucleotide residues 1-3 of SEQ ID NO:3) for the encoded endolysin is atg, and the termination codon for the encoded endolysin (nucleotide residues 856-858 of SEQ ID NO:3) is tga, taa or tag. Alternatively, the endolysin of C. acnes-specific bacteriophage φ50S may be encoded by the embodiment of SEQ ID NO:3 in which the start codon (nucleotide residues 1-3 thereof) for the encoded endolysin is gtg, and the termination codon for the encoded endolysin (nucleotide residues 856-858 of SEQ ID NO:3) is tga, taa or tag.

The endolysin protein encoded by SEQ ID NO:3 comprises the amino acid sequence (SEQ ID NO:4):

XRYIPAAHHS AGSNKPVNRV VIHATCPDVG FPSASRKGRA VSTANYFASP
SSGGSAHYVC DVGETVQCLS ESTIGWHAPP NPHSLGIEIC ADGGSHASFR
VPGHAYTREQ WLDPRVWPAV ERAAILCRQL CDKHGVPKRK LSVSDLKAGK
RGVCGHADVT DAWHQSDHDD PGPWFPWDRF MAVVCGGSGE SEELTVADVK
ALHDQIKQLS AQLSGSVNKL HHDVGVVQVQ NGDLGKRVDA LSWVKNPVTG
KLWRTKDALW SVWYYVLECR SRLDRLESAV NGLKK

wherein X at position 1 may be either the native valine, or may be methionine or leucine.

The endolysin of C. acnes-specific bacteriophage φ50S may alternatively be encoded without a precursor portion, so as to start from the start codon at position 15,666 of SEQ ID NO:1, and continue to the stop codon which begins at position 16,269 of SEQ ID NO:1 (as shown in underline above) (SEQ ID NO:5):

dtgggtatag agatttgcgc ggatgggggt tcgcacgcct cgttccgtgt
gccagggcat gcttacacga gggagcagtg gctggatcct cgggtgtggc
ccgccgtgga gcgtgccgcc atcctgtgtc ggcagttgtg tgacaagcat
ggtgttccga aaaggaagct tagcgtatcc gatttgaagg ccggtaaacg
tggtgtttgc gggcatgcgg atgttacgga tgcgtggcat cagtcggatc
atgacgatcc ggggccgtgg tttccgtggg acaggtttat ggccgtagtc
tgcggcggta gtggagagag tgaggagtta actgtggctg atgtgaaagc
cttgcatgat cagattaaac aattgtctgc tcagcttagt ggttcggtga
ataagctgca ccatgatgtt ggtgtggttc aggtacagaa tggtgatttg
ggtaagcgtg ttgacgccct gtcgtgggtg aagaatccgg tgacggggaa
gctgtggcgc accaaggatg ctttgtggag tgtctggtat tacgtgttgg
agtgtcgtag ccgtcttgac aggcttgagt ctgctgttaa cggtttgaaa
aagtrr

wherein: d at position 1 is either the native t, or may be a or g; and trr at positions 604-606 is either taa, tag, or tga.

More specifically, the endolysin of C. acnes-specific bacteriophage φ50S may be encoded by the embodiment of SEQ ID NO:5 in which the start codon for the encoded endolysin (nucleotide residues 1-3 of SEQ ID NO:5) is ttg, and the termination codon for the encoded endolysin (nucleotide residues 604-606 of SEQ ID NO:5) is tga, taa or tag. Alternatively, the endolysin of C. acnes-specific bacteriophage φ50S may be encoded by the embodiment of SEQ ID NO:5 in which the start codon (nucleotide residues 1-3 of SEQ ID NO:5) for the encoded endolysin is atg, and the termination codon for the encoded endolysin (nucleotide residues 604-606 of SEQ ID NO:5) is tga, taa or tag. Alternatively, the endolysin of C. acnes-specific bacteriophage φ50S may be encoded by the embodiment of SEQ ID NO:5 in which the start codon (nucleotide residues 1-3 thereof) for the encoded endolysin is gtg, and the termination codon for the encoded endolysin (nucleotide residues 604-606 of SEQ ID NO:5) is tga, taa or tag.

The endolysin protein encoded by SEQ ID NO:5 comprises the amino acid sequence (SEQ ID NO:6):

XGIEICADGG SHASFRVPGH AYTREQWLDP RVWPAVERAA ILCRQLCDKH
GVPKRKLSVS DLKAGKRGVC GHADVTDAWH QSDHDDPGPW FPWDRFMAVV
CGGSGESEEL TVADVKALHD QIKQLSAQLS GSVNKLHHDV GVVQVQNGDL
GKRVDALSWV KNPVTGKLWR TKDALWSVWY YVLECRSRLD RLESAVNGLK
K

wherein: X at position 1 may be either the native leucine, or may be methionine or valine.

SEQ ID NO:5 thus corresponds to residues 253-858 of SEQ ID NO:3. SEQ ID NO:6 thus corresponds to residues 86-285 of SEQ ID NO:4, with methionine replacing glycine at position 86 (underlined above).

The endolysin of C. acnes-specific bacteriophage φ50M is encoded starting from the start codon at position 15,414 of SEQ ID NO:2, to the stop codon which begins at position 16,269 of SEQ ID NO:2 (SEQ ID NO:7):

dtgagataca ttccagcggc gcatcactcg gccggatcaa ataagccggt
gaaccgtgtt gtgattcacg cgacatgccc ggatgtgggg tttccgtccg
cttcccgtaa ggggcgggcg gtgtctacag caaactattt tgcttcccca
tcgtcgggtg gttctgccca ttatgtgtgt gatgttgggg agacggtgca
gtgcttgtcg gagtctacga ttggttggca tgccccgccg aatccgcatt
ctttgggtat agagatttgc gcggatgggg gttcgcacgc ctcgttccgt
gtgccagggc atgcttacac gagggagcag tggctggatc ctcgggtgtg
gcccgccgtg gagcgtgccg ccatcctgtg tcggcagttg tgtgacaagc
atggtgttcc gaaaaggaag cttagcgtat ccgatttgaa ggccggtaaa
cgtggtgttt gcgggcatgc ggatgttacg gatgcgtggc atcagtcgga
tcatgacgat ccggggccgt ggtttccgtg ggacaggttt atggccgtag
tctgcggcgg tagtggagag agtgaggagt taactgtggc tgatgtgaaa
gccttgcatg atcagattaa acaattgtct gctcagctta gtggttcggt
gaataagctg caccatgatg ttggtgtggt tcaggtacag aatggtgatt
tgggtaagcg tgttgacgcc ctgtcgtggg tgaagaatcc ggtgacgggg
aagctgtggc gcaccaagga tgctttgtgg agtgtctggt attacgtgtt
ggagtgtcgt agccgtcttg acaggcttga gtctgctgtt aacggtttga
aaaagtrr

wherein: d at position 1 is either the native g, or may be a or t; and trr at positions 856-858 is either the native tga, or may be taa or tag.

More specifically, the endolysin of C. acnes-specific bacteriophage φ50M may be encoded by the embodiment of SEQ ID NO:7 in which the start codon for the encoded endolysin (nucleotide residues 1-3 of SEQ ID NO:7) is ttg, and the termination codon for the encoded endolysin (nucleotide residues 856-858 of SEQ ID NO:7) is tga, taa or tag. Alternatively, the endolysin of C. acnes-specific bacteriophage φ50M may be encoded by the embodiment of SEQ ID NO:7 in which the start codon (nucleotide residues 1-3 of SEQ ID NO:7) for the encoded endolysin is atg, and the termination codon for the encoded endolysin (nucleotide residues 856-858 of SEQ ID NO:7) is tga, taa or tag. Alternatively, the endolysin of C. acnes-specific bacteriophage φ50M may be encoded by the embodiment of SEQ ID NO:7 in which the start codon (nucleotide residues 1-3 thereof) for the encoded endolysin is gtg, and the termination codon for the encoded endolysin (nucleotide residues 856-858 of SEQ ID NO:7) is tga, taa or tag.

The endolysin protein encoded by SEQ ID NO:7 comprises the amino acid sequence of SEQ ID NO:4.

The endolysin of C. acnes-specific bacteriophage φ50M may alternatively be encoded starting from the start codon at position 15,666 of SEQ ID NO:2, to the stop codon which begins at position 16,269 of SEQ ID NO:2 (SEQ ID NO:8):

dtgggtatag agatttgcgc ggatgggggt tcgcacgcct cgttccgtgt
gccagggcat gcttacacga gggagcagtg gctggatcct cgggtgtggc
ccgccgtgga gcgtgccgcc atcctgtgtc ggcagttgtg tgacaagcat
ggtgttccga aaaggaagct tagcgtatcc gatttgaagg ccggtaaacg
tggtgtttgc gggcatgcgg atgttacgga tgcgtggcat cagtcggatc
atgacgatcc ggggccgtgg tttccgtggg acaggtttat ggccgtagtc
tgcggcggta gtggagagag tgaggagtta actgtggctg atgtgaaagc
cttgcatgat cagattaaac aattgtctgc tcagcttagt ggttcggtga
ataagctgca ccatgatgtt ggtgtggttc aggtacagaa tggtgatttg
ggtaagcgtg ttgacgccct gtcgtgggtg aagaatccgg tgacggggaa
gctgtggcgc accaaggatg ctttgtggag tgtctggtat tacgtgttgg
agtgtcgtag ccgtcttgac aggcttgagt ctgctgttaa cggtttgaaa
aagtrr

wherein: d at position 1 is either the native t, or may be a or g; and trr at positions 604-606 is either the native tga, or may be taa or tag.

More specifically, the endolysin of C. acnes-specific bacteriophage φ50M may be encoded by the embodiment of SEQ ID NO:8 in which the start codon for the encoded endolysin (nucleotide residues 1-3 of SEQ ID NO:8) is ttg, and the termination codon for the encoded endolysin (nucleotide residues 604-606 of SEQ ID NO:8) is tga, taa or tag. Alternatively, the endolysin of C. acnes-specific bacteriophage φ50M may be encoded by the embodiment of SEQ ID NO:8 in which the start codon (nucleotide residues 1-3 of SEQ ID NO:8) for the encoded endolysin is atg, and the termination codon for the encoded endolysin (nucleotide residues 604-606 of SEQ ID NO:8) is tga, taa or tag. Alternatively, the endolysin of C. acnes-specific bacteriophage φ50M may be encoded by the embodiment of SEQ ID NO:8 in which the start codon (nucleotide residues 1-3 thereof) for the encoded endolysin is gtg, and the termination codon for the encoded endolysin (nucleotide residues 604-606 of SEQ ID NO:8) is tga, taa or tag.

The endolysin protein encoded by SEQ ID NO:8 comprises the amino acid sequence of SEQ ID NO:6.

A preferred polynucleotide for expressing the endolysin of the C. acnes-specific bacteriophages φ50M and φ50S is (SEQ ID NO:30):

gcgggttcgg tggcctgtga gggtgtgaaa ccatcaccgg tggttaccgt
atcatcccac aagtaaaaga ggaagtgtgt tactagtgtt gatagtagtg

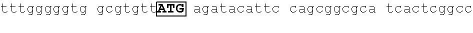

ggatcaaata agccggtgaa ccgtgttgtg attcacgcga catgcccgga
tgtggggttt ccgtccgctt cccgtaaggg gcgggcggtg tctacagcaa
actattttgc ttccccatcg tcgggtggtt ctgcccatta tgtgtgtgat
gttggggaga cggtgcagtg cttgtcggag tctacgattg gttggcatgc
cccgccgaat ccgcattctt tgggtataga gatttgcgcg gatgggggtt
cgcacgcctc gttccgtgtg ccagggcatg cttacacgag ggagcagtgg
ctggatcctc gggtgtggcc cgccgtggag cgtgccgcca tcctgtgtcg
gcagttgtgt gacaagcatg gtgttccgaa aaggaagctt agcgtatccg
atttgaaggc cggtaaacgt ggtgtttgcg ggcatgcgga tgttacggat
gcagttgtgt agtcggatca tgacgatccg gggccgtggt ttccgtggga
caggtttatg gccgtagtct gcggcggtag tggagagagt gaggagttaa
ctgtggctga tgtgaaagcc ttgcatgatc agattaaaca attgtctgct
cagcttagtg gttcggtgaa taagctgcac catgatgttg gtgtggttca
ggtacagaat ggtgatttgg gtaagcgtgt tgacgccctg tcgtgggtga
agaatccggt gacggggaag ctgtggcgca ccaaggatgc tttgtggagt
gtctggtatt acgtgttgga gtgtcgtagc cgtcttgaca ggcttgagtc

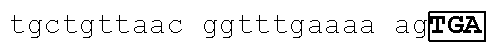

Optionally, the polynucleotide of SEQ ID NO:30 may additionally comprise a polynucleotide linker, such as tggtggtttgtt (SEQ ID NO:31) after the 3′ terminus thereof. The presence of such linker facilitates the expression of polynucleotides that may be positioned 3′ to an endolysin-encoding polynucleotide.

Holin proteins assemble to form pores in the cellular membrane, and thereby facilitate the lytic activity of the phage endolysin (Farrar, M. D. et al. (2007) “Genome Sequence and Analysis of a Propionibacterium acnes Bacteriophage,” J. Bacteriol. 189(11):4161-4167). Thus, the present invention additionally comprises pharmaceutical compositions that comprise the C. acnes-specific bacteriophages φ50M and φ50S holin protein. The C. acnes-specific bacteriophages φ50M and φ50S additionally encode an identical holin protein (SEQ ID NO:9):

XGKQFWLGLL ERAAKTFVQT FVAVLGVTAG VTYTAESFRG LPWESALITA
TVAAVLSVAT SFGSPSFVAG KPGKPQLDAG LVPPDDPGIV EPHSVDVSDP
GMIEPMDEAD VAGYVPKRAA ESEVGTVEST VA

wherein: X at position 1 may be either the native valine or may be methionine or leucine.

The C. acnes-specific bacteriophage φ50M and φ50S holin protein (SEQ ID NO:9) may be encoded by (SEQ ID NO:10):

dtgggtaaac agttttggtt aggtttactg gagcgggcgg ctaagacttt
tgtgcaaacg tttgttgctg tgttgggggt gacggcgggt gtcacctata
cggcggagtc gtttcgcggt ttgccgtggg agtcggccct gataacagct
acggtggctg cggtgttgtc ggtggctaca tcgtttggta gcccgtcgtt
tgtggccggc aagcccggca agcctcagct ggatgcgggt ttggttccac
cggatgatcc cggaatagtg gagcctcatt cggtggatgt gtcggatcct
ggcatgatcg agccgatgga tgaggctgat gttgccggct atgtgccgaa
gcgtgccgcc gagtcggagg ttggcacggt agagtctact gttgcatrr

wherein: d at position 1 is either the native g, or may be a or t; and trr at positions 397-399 is either the native taa, or may be tag or tga.

More specifically, the holin protein of C. acnes-specific bacteriophage φ50S may be encoded by the embodiment of SEQ ID NO: 10 in which the start codon for the encoded holin protein (nucleotide residues 1-3 of SEQ ID NO:10) is ttg, and the termination codon for the encoded endolysin (nucleotide residues 397-399 of SEQ ID NO:10) is tga, taa or tag. Alternatively, the holin protein of C. acnes-specific bacteriophage φ50S may be encoded by the embodiment of SEQ ID NO:10 in which the start codon (nucleotide residues 1-3 of SEQ ID NO:10) for the encoded holin protein is atg, and the termination codon for the encoded holin protein (nucleotide residues 397-399 of SEQ ID NO:10) is tga, taa or tag. Alternatively, the holin protein of C. acnes-specific bacteriophage φ50S may be encoded by the embodiment of SEQ ID NO:10 in which the start codon (nucleotide residues 1-3 thereof) for the encoded holin protein is gtg, and the termination codon for the encoded holin protein (nucleotide residues 397-399 of SEQ ID NO:10) is tga, taa or tag.

In one embodiment, the invention contemplates therapeutic compositions that comprise the above-described endolysin, the above-described holin protein or both such proteins. Such proteins can be produced through the recombinant expression of the aboe-described polynucleotides. In one embodiment, such proteins can be produced through the recombinant expression of a single polynucleotide that encodes both such proteins. An example of such a polynucleotide is (SEQ ID NO:32):

gcgggttcgg tggcctgtga gggtgtgaaa ccatcaccgg tggttaccgt
atcatcccac aagtaaaaga ggaagtgtgt tactagtgtt gatagtagtg

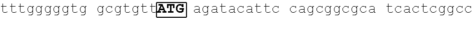

ggatcaaata agccggtgaa ccgtgttgtg attcacgcga catgcccgga
tgtggggttt ccgtccgctt cccgtaaggg gcgggcggtg tctacagcaa
actattttgc ttccccatcg tcgggtggtt ctgcccatta tgtgtgtgat
gttggggaga cggtgcagtg cttgtcggag tctacgattg gttggcatgc
cccgccgaat ccgcattctt tgggtataga gatttgcgcg gatgggggtt
cgcacgcctc gttccgtgtg ccagggcatg cttacacgag ggagcagtgg
ctggatcctc gggtgtggcc cgccgtggag cgtgccgcca tcctgtgtcg
gcagttgtgt gacaagcatg gtgttccgaa aaggaagctt agcgtatccg
atttgaaggc cggtaaacgt ggtgtttgcg ggcatgcgga tgttacggat
gcgtggcatc agtcggatca tgacgatccg gggccgtggt ttccgtggga
caggtttatg gccgtagtct gcggcggtag tggagagagt gaggagttaa
ctgtggctga tgtgaaagcc ttgcatgatc agattaaaca attgtctgct
cagcttagtg gttcggtgaa taagctgcac catgatgttg gtgtggttca
ggtacagaat ggtgatttgg gtaagcgtgt tgacgccctg tcgtgggtga
agaatccggt gacggggaag ctgtggcgca ccaaggatgc tttgtggagt
gtctggtatt acgtgttgga gtgtcgtagc cgtcttgaca ggcttgagtc

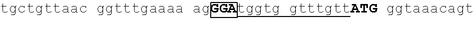

tttggttagg tttactggag cgggcggcta agacttttgt gcaaacgttt
gttgctgtgt tgggggtgac ggcgggtgtc acctatacgg cggagtcgtt
tcgcggtttg ccgtgggagt cggccctgat aacagctacg gtggctgcgg
tgttgtcggt ggctacatcg tttggtagcc cgtcgtttgt ggccggcaag
cccggcaagc ctcagctgga tgcgggtttg gttccaccgg atgatcccgg
aatagtggag cctcattcgg tggatgtgtc ggatcctggc atgatcgagc
cgatggatga ggctgatgtt gccggctatg tgccgaagcg tgccgccgag
tcggaggttg gcacggtaga gtctactgtt gcaTAA

In SEQ ID NO:32, the linker betweeen the endolysin gene and the holin gene (shown underlined) may be the endogenous linker: tggtggtttgtt (SEQ ID NO:31), or may be an alternative polynucleotide of similar length.

Optionally, the polynucleotide of SEQ ID NO:32 may additionally comprise a further polynucleotide, such as SEQ ID NO:33 after the 3′ terminus thereof. The presence of such further polynucelotide facilitates the expression of the encoded polynucleotides and additional polynucleotides that may be positioned 3′ thereto.

SEQ ID NO: 33:
gtgaatatag atgtgtgccc cagcggtgct gccacgatcg tgtggtggtt
gccgctgggg cactattttt gtgtctatag tatt

### II. Pharmaceutical Compositions

The pharmaceutical compositions of the present invention comprise prophylactically effective or therapeutically effective amounts of C. acnes-specific bacteriophage φ50S and/or (′. acnes-specific bacteriophage φ50M, or the endolysin of such bacteriophages. In a further embodiment, pharmaceutical compositions of the present invention may comprise C. acnes-specific bacteriophage φ50S and/or C. acnes-specific bacteriophage φ50M in combination with the endolysin of such bacteriophages. Such compositions may be used alone or may be further combined with other antimicrobial agents or with immunomodulators.

As used herein, a “prophylactically effective amount” is an amount of such bacteriophage-containing and/or endolysin-containing compositions capable of decreasing the probability of future infection in a subject (e.g., a human) at risk of a C. acnes infection. The bacteriophage compositions of the present invention may be used prophylactically as a prebiotic or probiotic additive. As used herein, a “therapeutically effective amount” is an amount of such bacteriophage-containing compositions and/or endolysin-containing compositions capable of treating acne, promoting wound healing, inhibiting the growth of biofilms or preventing or treating surgical related infections in a subject.

As used herein, the term “treating” denotes lessening the severity or duration of an infection, including by resolving or eliminating such infection. As used herein, the term “promoting wound healing” denotes accelerating the process of wound healing so as to decrease the severity of a wound or its duration. As used herein, the term “inhibiting the growth of biofilms” denotes slowing the rate of biofilm progression or the size, location or invasiveness of a biofilm.

The bacteriophage compositions of the present invention can be prepared in sterile, preserved, buffered suspensions to form “therapeutic compositions” or “prophylactic compositions, as the case may be. Such compositions may be lyophilized for extended storage and reconstituted before use, and formulated for administration as a pharmaceutical composition.

The pharmaceutical compositions of the present invention may further comprise one or more of a variety of additional pharmaceutically acceptable components. See REMINGTON: THE SCIENCE AND PRACTICE OF PHARMACY (21st Edition) (2005) (Troy, D. B. et al. (Eds.) Lippincott Williams & Wilkins (Publs.), Baltimore MD), which is hereby incorporated by reference in its entirety. The preferred pharmaceutical compositions of the present invention depend on the intended mode of administration and therapeutic application. The pharmaceutical compositions of the present invention can also include, depending on the intended mode of administration and therapeutic application, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition of the present invention, and which are vehicles commonly used to formulate pharmaceutical compositions for animal or human administration. The diluent is selected to not to affect the biological activity of the combination. Examples of such diluents are distilled water, physiological phosphate-buffered saline, Ringer's solutions, dextrose solution, and Hank's solution. In addition, the pharmaceutical compositions of the present invention may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The pharmaceutical compositions of the present invention may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of the pharmaceutical compositions of the present invention is determined based on the lack of significant negative impact on the desired biological properties of the chosen compound on the bacteriophage of the pharmaceutical compositions of the present invention (e.g., less than a substantial impact (e.g., 10% or less relative inhibition, 5% or less relative inhibition, etc.).

The pharmaceutical compositions of the present invention may also comprise pharmaceutically acceptable antioxidants, for example: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more preservatives, wetting agents, emulsifying agents, dispersing agents, preservatives or buffers, which may enhance the shelf life or effectiveness of the pharmaceutical compositions of the present invention. The therapeutic compositions of the present invention may be prepared with carriers that will protect the composition against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the pharmaceutical compositions of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, microemulsion, liposome, or other ordered structure suitable to high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions may comprise petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient. An exemplary composition comprises an scFv at about 5 mg/mL, formulated in aqueous buffer consisting of 50 mM L-histidine, 150 mM NaCl, adjusted to pH 6.0 with HCl.

The preparation also can be emulsified or encapsulated in liposomes or micro particles, such as polylactide, polyglycolide, or copolymer, for enhanced adjuvant effect (Langer R. (1990) “New Methods Of Drug Delivery,” Science 249(4976): 1527-1533; Hanes, J. et al. (1997) “New Advances In Microsphere-Based Single-Dose Vaccines,” Adv. Drug Del. Rev. 28(1):97-119, which are hereby incorporated by reference in their entirety).

### III. Methods of Administration

The pharmaceutical compositions of the present invention can be administered to a subject by parenteral, topical, transdermal, intramuscular, intravenous, intraarterial, infusion, subcutaneous, perfusion, oral or other means for prophylactic and/or therapeutic treatment. In some methods, the therapeutic compositions of such formulations are administered as a sustained release composition or device (including a bandage, patch, etc. In some methods, the pharmaceutical compositions of the present invention are applied to, or injected directly into, a particular tissue where C. acnes infection is observed, suspected or considered likely to occur. In some methods, the therapeutic compositions of the present invention are applied (as, for example, by a rinse or coating) to medical devices, implant devices, stents, bandages, medical gauze, suturing materials, clamps, etc., which have been introduced, or are subsequently introduced, into a subject in the course of a surgery or other procedure (i.e., at a pre- or post-surgical site). The C. acnes therapeutic genes may also be expressed using various vectors including viral vectors.

Effective doses of the pharmaceutical compositions of the present invention, for the treatment of the above-described conditions may vary depending upon many different factors, including means of administration, target site, physiological state of the patient, other medications administered, and whether treatment is prophylactic or therapeutic. For example, a prophylactically effective amount of a cream or ointment for topical administration may comprise from about 1×10⁶ to about 1×10⁹ or more PFU per gram of such pharmaceutical composition. A therapeutically effective amount of a cream or ointment for topical administration may, for example, comprise from about 1×10⁷ to about 1×10¹⁰ or more PFU per gram of such pharmaceutical composition. Such treatment dosages are typically adjusted to optimize their safety and efficacy. A physician having ordinary skill in the art may readily determine and prescribe the effective amount of the pharmaceutical compositions required. For example, treatment may be initiated with an initial dosage of a pharmaceutical composition of the present invention that is increased or decreased until the desired effect is achieved. Likewise, treatment may be initiated with an initial administration frequency that is increased or decreased until the desired effect is achieved. Such modifications of dosage and of frequency of administration may be combined, for example, to provide higher or lower dosages at increased frequency, or at decreased frequency as desired to provide effective treatment or prophylaxis. In general, a suitable dose of a composition of the present invention will be daily, every 2 days, every 3 days, weekly, every 2 weeks, monthly, every 2 to 6 months, etc., although more frequent or less frequent administrations may be employed. The dosage and frequency of administration can vary depending on whether the treatment is prophylactic or therapeutic. In prophylactic applications, a relatively low dosage of a pharmaceutical composition of the present invention may be administered at relatively infrequent intervals over a long period of time. Some patients may continue to receive treatment for the rest of their lives. In therapeutic applications, a relatively higher dosage of a pharmaceutical composition of the present invention at greater frequency is sometimes desired until progression of disease is reduced or terminated, and preferably until the patient shows partial or complete amelioration of symptoms of the disease. Thereafter, the patient can be administered such pharmaceutical composition using a prophylactic dosage regime.

The pharmaceutical composition of the present invention may comprise a bacteriophage or a bacteriophage endolysin, endolysin and holin and one or more additional therapeutic agents (e.g., a second bacteriophage, an antibiotic, an anti-steroidal agent, a second endolysin, etc.), so as to provide a combination therapy.

### EXAMPLES

Having now generally described the invention, the same will be more readily understood through reference to the following examples, which are provided by way of illustration and are not intended to be limiting of the present invention unless specified.

### Example 1

### Isolation of C. acnes-specific Bacteriophages

Cutibacterium acnes-specific bacteriophages were isolated from Cutibacterium acnes nine of seventeen clinical sample collections of Integrated Pharma Services (IPS) Biorepository. Each strain from the Biorepository's glycerol stocks was streaked onto reinforced clostridial medium (RCM, Oxoid) agar plates and incubated at 37ºC for 48 hours under anaerobic condition to form a seed plate. Using an inoculating loop, 10 colonies from the seed plate were inoculated into 5 ml of double-strength RCM broth including sodium thioglycolate (2× RCM broth with 1× sodium thioglycolate) and grown anaerobically at 37° C. for 48 hours at 200 rpm. The cultures were centrifuged (10,000×g for 10 minutes) to remove bacterial ells and the supernatants were filtered through 0.22 μm filter (Millipore). A top agar overlay assay was conducted with the filtrates to isolate plaques. The 2-day old culture (optical density at 600 nm=>1.5) of clindamycin-sensitive (′. acnes ATCC 6919 as a host strain was mixed with 50 μl of the filtrate and then added to 5 ml of RCM top agar (0.7%). The tube was inverted 20 times to mix well and was then poured onto the surface of an RCM agar plate. These steps were repeated for each filtrate. All plates were then incubated anaerobically for 48 hours at 37° C. The presence of phage was identified by plaque formation on the bacterial lawn.

### Example 2

### Purification and Propagation of C. acnes-specific Bacteriophages

Individual plaques were excised carefully to avoid possible contamination and were placed into 500 μl of RCM broth and vortexed to solubilize the phage into the broth. The supernatant was centrifuged at 10,000×g for 10 minutes, and then serially 10-fold diluted, introduced into 5 ml of RCM top agar (0.7%) and applied to an RCM agar plate with C. acnes, and cultured as above in order to obtain single plaques. This purification process was repeated 4 or 5 times to ensure that each plaque came from one virion infection. Each of the isolated phages was propagated on the host strain to achieve a phage stock having a concentration of approximately 10¹⁰ PFU/ml. Through this purification and propagation process, the lytic activity was determined by a spot assay in which 10 or 20 μl of the filtrate or supernatant was placed onto a lawn of the C. acnes ATCC 6919 strain that was cultured for 48 hours (optical density at 600 nm=>1.5). Nine different bacteriophage isolates, designated: φ044, φ86, φ11828, φ59, φ50M, 50S, φ86L, φ86S, and φ50L, were successfully purified and propagated to produce preparations harboring approximately 10⁹ PFU/ml (Table 1). Phage titer was determined by both spot assay and top agar overlay assay.

TABLE 1
Cutibacterium acnes-Specific Bacteriophage Isolates

| No. | Bacteriophages |
|---|---|
| 1 | φ044 |
| 2 | φ86 |
| 3 | φ11828 |
| 4 | φ59 |
| 5 | φ50M |
| 6 | φ50S |
| 7 | φ86L |
| 8 | φ86S |
| 9 | φ50L |

### Example 3

### Cloning and Modification of the Bacteriophage Ø50S Endolysin Gene

The endolysin gene of the C. acnes-specific bacteriophage Ø50S was cloned and mutated using nested PCR Primer Sets that were designed over Ø50S (“phiPA50S”) genomic sequences flanking N-terminal 116 nucleotides (nt) and C-terminal 209 nt of the endolysin gene, which starts at 117 nt and ends 974 nt. The employed primers are described in Table 2A and Table 2B.

TABLE 2A
Sequences of Primers for Cloning of Endolysin Gene (phiPA50S)

| SEQ | | |
|---|---|---|
| ID | | |
| NO | Primer Name | Sequence (5′-3′) |
| 11 | 50S_1F | ACC GTA TCA TCC CAC AAG TAA A |
| 12 | 50S_1R | GAA ACG ACT CCG CCG TAT AG |
| 13 | 50S_2F | GGG TGG CGT GTT GTG AGA TA |
| 14 | 50S_2R | CTG TTA TCA GGG CCG ACT CC |
| 15 | 50S_2F-NcA | GGG TGG CGT ACC ATG GGA TA |
| 16 | 50S_2F-NcB | GGG TGG CGT ACC ATG GGA TAC ATT |
| 17 | 50S_3R | AAC AAA CCA CCA TCA CTT TTT CAA AC |
| 18 | 50S_3R-St | AAC AAA CCA CCA TCC CTT TTT CAA AC |
| 19 | UniF | GGT GGC CTG TGA GGG TGT GAA A |

TABLE 2B
Characteristics of Primers for Cloning
of Endolysin Gene (phiPA50S)

| SEQ | Primer | Length | G + C | Tm | Start | Stop |
|---|---|---|---|---|---|---|
| ID NO | Name | (bp) | (%) | (° C.) | (nt) | (nt) |
| 11 | 50S_1F | 22 | 40.9 | 53.7 | 45 | 67 |
| 12 | 50S_1R | 20 | 55 | 55.2 | 1102 | 1081 |
| 13 | 50S_2F | 20 | 55 | 57.3 | 105 | 124 |
| 14 | 50S_2R | 20 | 60 | 57.4 | 1134 | 1115 |
| 15 | 50S_2F-NcA | 20 | 60 | 59.2 | 105 | 124 |
| 16 | 50S_2F-NcB | 24 | 54.2 | 61 | 105 | 128 |
| 17 | 50S_3R | 26 | 34.6 | 55.2 | 960 | 986 |
| 18 | 50S_3R-St | 26 | 38.5 | 56.8 | 960 | 986 |
| 19 | UniF | 22 | 59.1 | 61.9 | 9 | 31 |

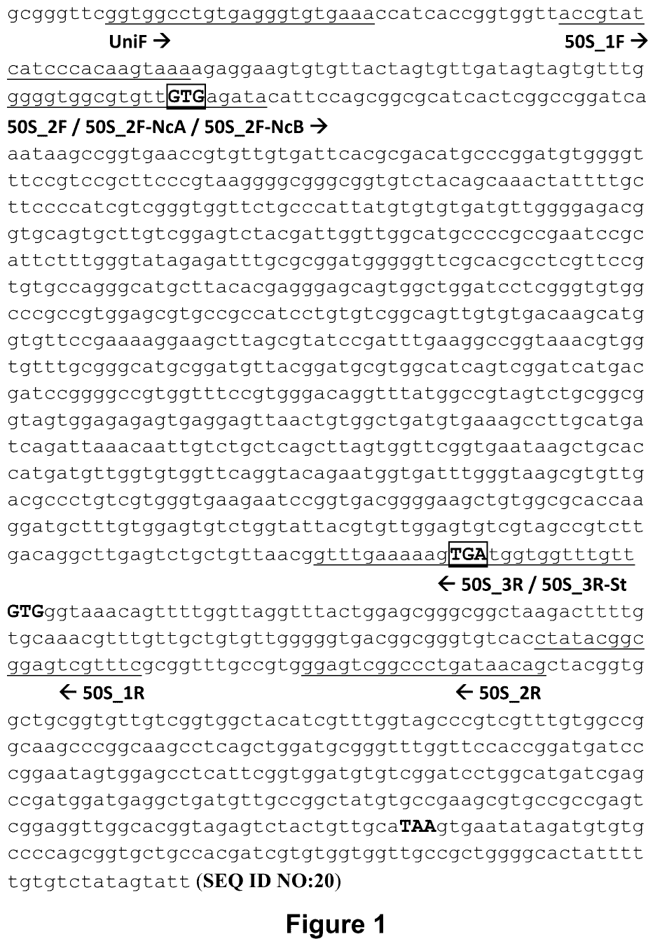

The primer sets: UniF and 50S_3R are universal primers that amplify the entire C. acnes endolysin gene. Primer 50S_2 F contains the native start codon ‘GTG’ of the protein. Primer 50S_2 F-NcA introduces an Ncol enzyme (C/CATGG) site (underlined) and causes the start codon of the protein to become ATG. Use of the 50S_2 F-NcB primer results in the addition of 4 base pairs. Use of the 50S_3R-St primer results in the replacement of the native TGA stop codon with a TTA stop codon. The native stop codon was replaced while preserving the reading frame through the C-terminal tag (V5 epitope and poly-histidine region). The 50S_1 F and 50S_3R-St primer sets are used to produce an amplified product that comprises a V5 epitope and a poly-histidine region. The 50S_2 F-NcA/B and 50S_3R-St primers are used to remove an N-terminal leader (EK) from the amplified product and to include the V5 epitope and poly-histidine region in that product. The 50S_2 F_NcA/B and 50S_3R primers are used to remove the N-terminal leader (EK). The positioning of the employed primers is illustrated in FIG. 1.

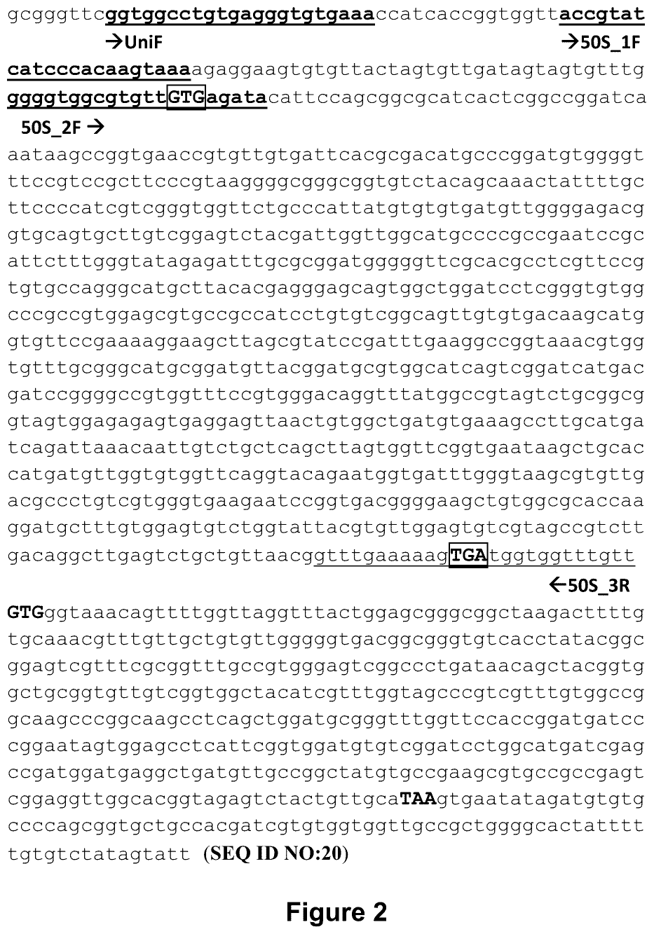

FIG. 2 shows the orientation and positioning of the PCR primers UniF, 50S_1 F, 50S_2 F and Primer 50S_3R used to subclone and modify the phiPA50S endolysin gene. The forward primers UniF and 50S_1 F (both underlined) were designed to cover an upstream region if a native promoter was desired to express the endolysin. Primer 50S_2 F (underlined) includes the start codon GTG (boxed, uppercase). Primer 50S_3R (underlined) includes the stop codon TGA (boxed, uppercase).

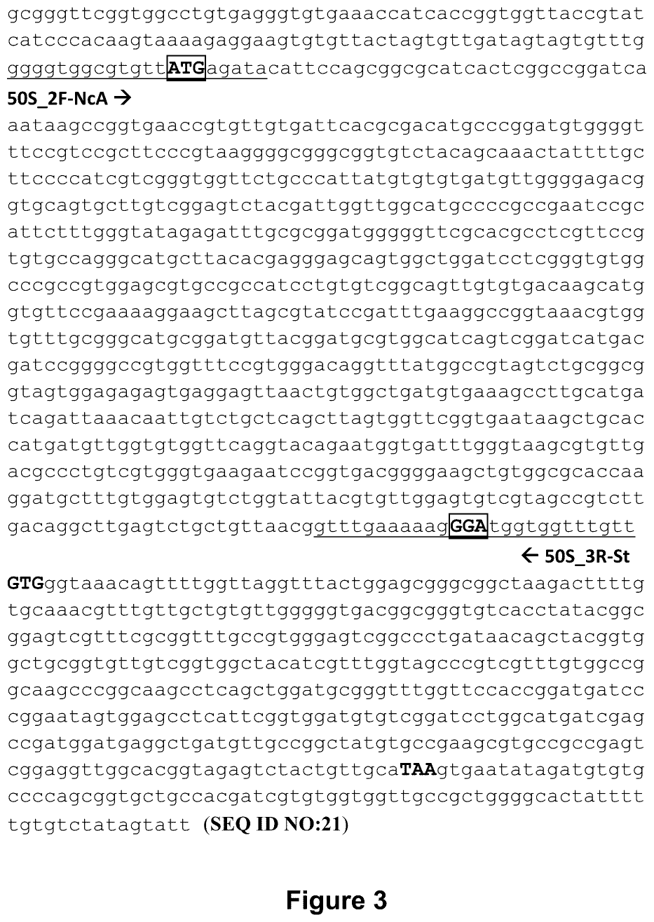

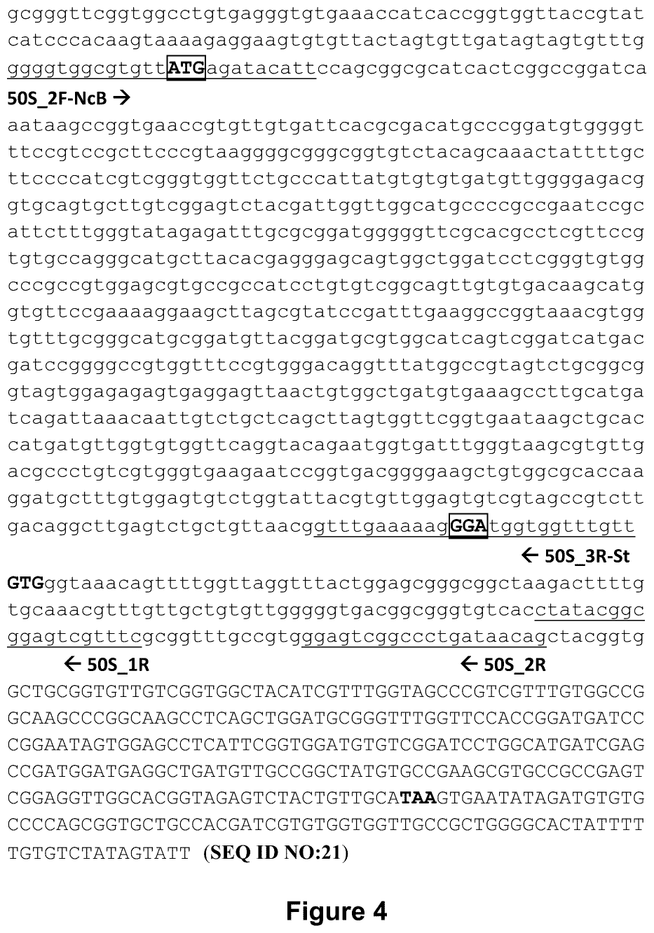

The endolysin gene of the C. acnes-specific bacteriophage 50S was subcloned into the pBAD-TOTO TA vector (ThermoFisher) using either primer 50S_2 F-NcA or primer 50S_2 F-NcB, which change the start codon to ATG, and primer 50S_3R-St, which includes the stop codon change to GGA. Primer 50S_2 F-NcB is four nucleotides longer than primer 50S_2 F-NcA. It can disrupt the stop codon and be continuously expressed with a 6× His tag. The positioning of the employed primers is illustrated in FIG. 3 (for primer 50S_2 F-NcA and primer 50S_3R-St) and FIG. 4 (for primer 50S_2 F-NcB and primer 50S_3R-St, primer 50S_1R or primer 50S_2R).

### Example 4

### Cloning and Modification of the Bacteriophage Ø50S Holin Gene

The holin gene of the C. acnes-specific bacteriophage φ50S was cloned and mutated using nested PCR Primer Sets that were designed over φ50S (“phiPA50S”) genomic sequences flanking N-terminal 58 nucleotides (nt) and C-terminal 71 nts of the Holin gene, which starts 1 nt and ends 399 nt. The employed primers are described in Table 3A and Table 3B.

TABLE 3A
Sequences of Primers for Cloning of Holin Gene (phiPA50S)

| SEQ | | |
|---|---|---|
| ID | | |
| NO | Primer Name | Sequence (5′-3′) |
| 22 | 50S_H1F | CCG TCT TGA CAG GCT TGA GT |
| 23 | 50S_H1R | CAA AAA TAG TGC CCC AGC GG |
| 24 | 50S_H2F | TGG TGG TTT GTT GTG GGT AA |
| 25 | 50S_H2F-NcA | TGG TGG TTT ACC ATG GGT AA |
| 26 | 50S_H2R | TCT ATA TTC ACT TAT GCA ACA GTA GA |
| 27 | 50S_H2R-St | TCT ATA TTC ACT TCT GCA ACA GTA GA |

TABLE 3B
Characteristics of Primers for Cloning of Holin Gene (phiPA50S)

| SEQ | Primer | Length | G + C | Tm | Start | Stop |
|---|---|---|---|---|---|---|
| ID NO | Name | (bp) | (%) | (° C.) | (nt) | (nt) |
| 22 | 50S_H1F | 20 | 55.0 | 59.9 | −58 | −39 |
| 23 | 50S_H1R | 20 | 55.0 | 59.8 | 451 | 470 |
| 24 | 50S_H2F | 20 | 45.0 | 54.6 | −12 | 8 |
| 25 | 50S_H2F-NcA | 20 | 45.0 | 53.7 | −12 | 8 |
| 26 | 50S_H2R | 26 | 30.8 | 51.6 | 385 | 410 |
| 27 | 50S_H2R-St | 26 | 34.6 | 53.4 | 385 | 410 |

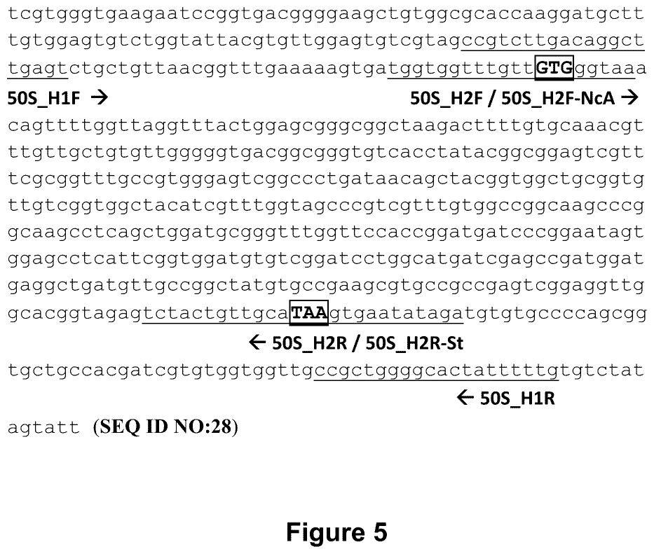

Primer 50S_H2F contains the native start codon ‘GTG’ of the protein. Primer 50S_H2F-NcA introduces an Ncol enzyme (AC/CATGG; SEQ ID NO:20) site (underlined) and causes the start codon of the protein to become ATG. Use of primer 50S_H2R maintains the native TAA stop codon. Use of primer 50S_H2R-St results in the replacement of the native TAA stop codon with a GAA codon. The native stop codon was replaced while preserving the reading frame through the C-terminal tag (V5 epitope and poly-histidine region). The 50S_H1F and 50S_2R-St primer sets are used to produce an amplified product that comprises a V5 epitope and a poly-histidine region. The 50S_H2F-NcA and 50S_H2R-St primers are used to remove an N-terminal leader (EK) from the amplified product and to include the V5 epitope and poly-histidine region in that product. The 50S_H2F_NcA and 50S_H2R primers are used to remove the N-terminal leader (EK). Primer 50S_HIR is positioned after the holin gene stop codon. The positioning of the employed primers is illustrated in FIG. 5.

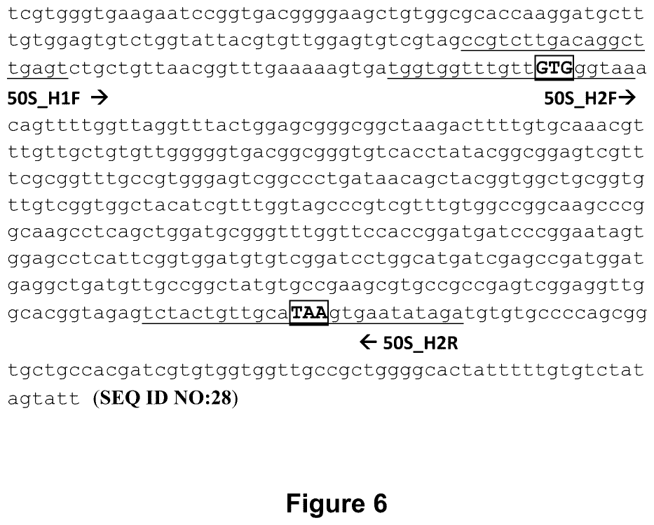

FIG. 6 shows the orientation and positioning of forward primers 50S_H1F, 50S_H2F and a reverse primer 50S_H2R used to subclone and modify the phiPA50S holin gene. The forward primer, 50S_H1F (underlined), was designed to cover an upstream region if a native promoter was desired to express the holin protein. Primer 50S_H2F (underlined) includes the start codon GTG (boxed, uppercase). Primer 50S_H2R (underlined) includes the stop codon TAA (boxed, uppercase).

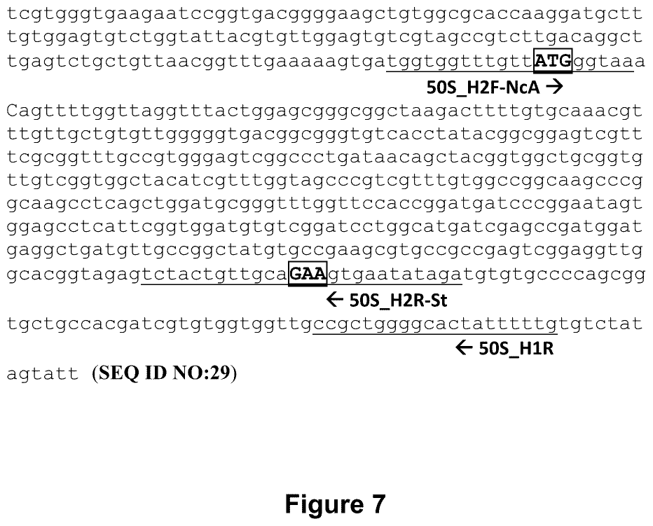

FIG. 7 shows the orientation and positioning of forward primer 50S_H2F_NcA and reverse primers 50S_H2R-St and 50S_HIR used to subclone and modify the phiPA50S holin gene. As stated above, the forward primer, 50S_H2F-NcA (underlined) introduces an Ncol cleavage site and replaces the native start codon with odon ATG. Reverse primer 50S_H2R-St replaces the native TAA stop codon with a GAA codon.

### Example 5

### Lytic Activity of 9 C. Acnes-Specific Bacteriophages

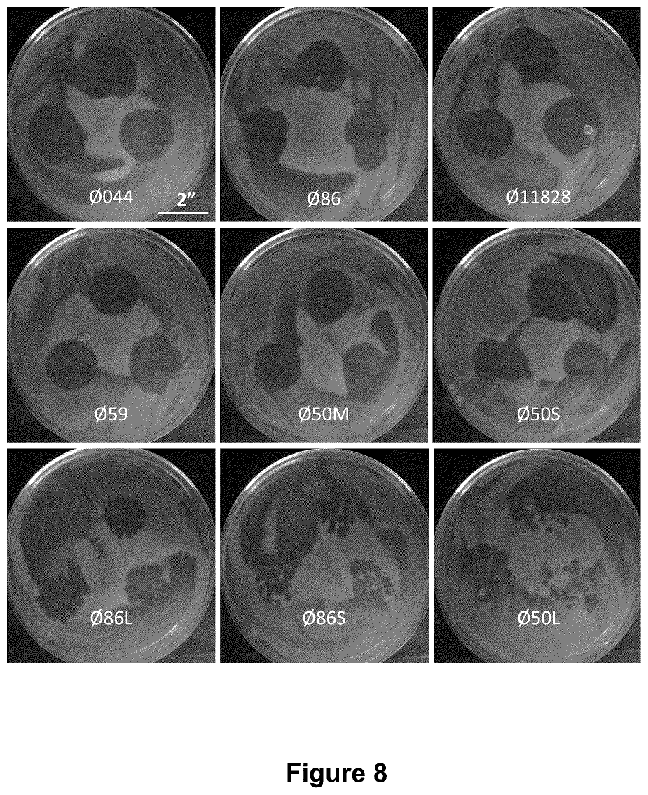

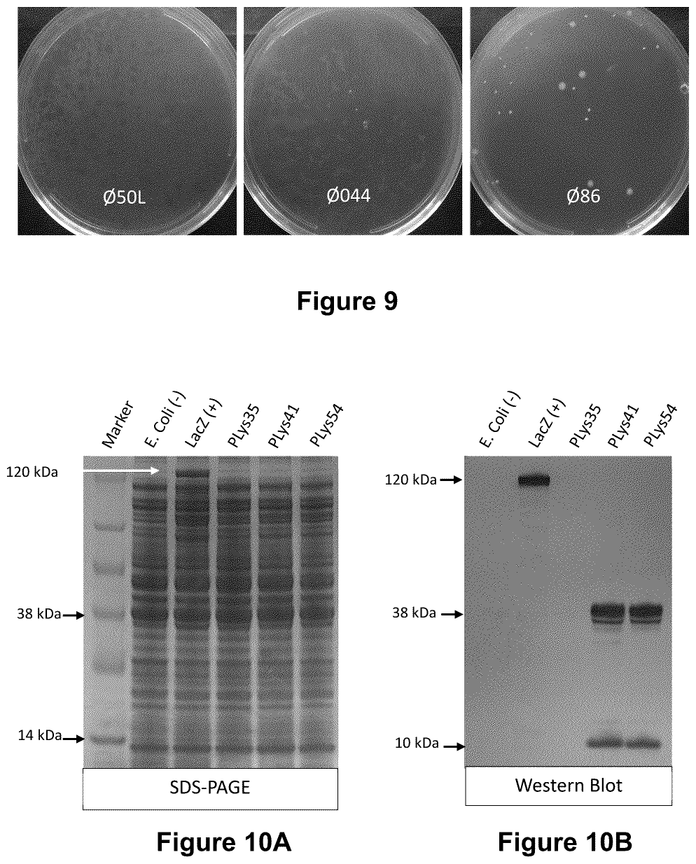

The titers of the nine C. acnes-specific bacteriophages were calculated via a propagation process, and their lytic activities against C. acnes ATCC 6919 strain was confirmed (FIG. 8). C. acnes ATCC 6901 cultures were prepared approximately 10⁸ CFU/ml and plated onto the RCM agar plate. A 20 μl of each of 9 phages was spot in triplicate onto the agar plates. After 48 hours incubation under anaerobic condition, a distance from zone of inhibition (ZOI) was measured. About 2 inches of ZOI was observed for 7 bacteriophages (φ044, φ86, φ11828, φ59, φ50S, φ50S, φ86L) against C. acnes ATCC 6919 strains. Three phages φ86L, φ86S, and φ50L showed different images of zone of inhibition. All of 9 bacteriophages have good lytic activity against C. acnes ATCC 6919 as a host strain. Furthermore, three bacteriophages (+50L, 044, and @86) having MOI=2 were selected and evaluated using a top agar overlay assay against C. acnes ATCC 6919. A bacteriophage Ø86 lysed and eradicated completely C. acnes ATCC 6919 cultures. And two other bacteriophages φ50L and ¢044 were mostly lysed the host bacterial cells as shown in the FIG. 9.

### Example 6

### C. Acnes-Specific Efficacy Testing of 9 Bacteriophages

As many as 42 C. acnes isolates were tested for nine C. acnes-specific bacteriophages. According to the method and procedure of Zone of Inhibition (ZOI) assay (see, e.g., Hudzicki, J. (2009) “Kirby-Bauer Disk Diffusion Susceptibility Test Protocol,” Amer. Soc. Microbiol. Monograph:1-23), 20 μl of approximately 10⁹ PFU/ml of each of 9 bacteriophages was applied onto RCM agar plates, which had been plated with 42 different C. acnes cultures. After 48 hours incubation at 37° C. under anaerobic condition, ZOI was measured and recorded as shown in Table 4. Nine C. acnes-specific bacteriophages showed effectiveness against all of the 42 C. acnes isolates, though the distance of ZOI have varied depending on the isolates.

TABLE 4

| | C. acnes | Distance of ZOI for 9 Phages (Inches) | | | | | | | | |
|---|---|---|---|---|---|---|---|---|---|---|
| No. | Strain | φ044 | φ86 | φ11828 | φ59 | φ50M | φ50S | φ86L | φ86S | φ50L |
| 1 | 122 | 1 | 1 | 1 | 1 | 1 | 1 | 1 | 1 | 1 |
| 2 | 253 | 1 | 1 | 1.5 | 1.5 | 1 | 1 | 1.5 | 1.5 | 1.5 |
| 3 | 489 | 1 | 1.5 | 2 | 1.5 | 1 | 2 | 2 | 1 | >2 |
| 4 | 490 | 1 | 1 | 1 | 1 | 1 | 1 | 1 | 1 | 1.5 |
| 5 | 491 | 1 | 1.5 | 1.5 | 1.5 | 1 | 1 | 1 | 1 | 1 |
| 6 | 493 | >2 | 1 | 1 | 1 | >2 | 2 | 1 | 1 | >2 |
| 7 | 494 | 2 | 1 | 1.5 | 1.5 | 1 | 1 | 1 | 1 | 1 |
| 8 | 495 | 2 | 2 | 1.5 | 1.5 | 1.5 | 1 | 1.5 | 2 | 1.5 |
| 9 | 496 | 1 | 1 | 1 | 1 | 1 | 1 | 1 | 2 | 1 |
| 10 | 497 | 1 | 1.5 | 1.5 | 2 | 2 | 1 | 2 | 1.5 | 1 |
| 11 | 498 | 1 | 1 | 1.5 | 1 | 1 | 1 | 1.5 | 2 | 2 |
| 12 | 499 | 1.5 | 2 | 1.5 | 1.5 | 1 | 1 | 1 | 1 | 1.5 |
| 13 | 500 | 1 | 2 | 1 | 1 | 1 | 1 | 1 | 1.5 | 1 |
| 14 | 501 | 0.5 | 0.5 | 0.5 | 0.5 | 0.1 | 0.5 | 0.5 | 0.5 | 0.5 |
| 15 | 502 | 1.5 | 1.5 | 1 | 1 | 1 | 1 | 2 | 2 | 2 |
| 16 | 503 | 2 | 1 | 2 | 2 | 1.5 | 1 | 1.5 | 2 | 1 |
| 17 | 504 | 1 | 1 | 1.5 | 1 | 2 | 1 | 1 | 2 | 2 |
| 18 | 505 | 1 | 1 | 1 | 1 | 1 | 1 | 1 | 1 | 1 |
| 19 | 506 | 1 | 0.2 | 0.5 | 0.5 | 0.5 | 0.5 | 1 | 1 | 1 |
| 20 | 507 | 2 | 2 | 2 | 2 | 1.5 | 1.5 | 1.5 | 2 | 1.5 |
| 21 | 508 | 2 | 0.5 | 1 | 0.5 | 1.5 | 1.5 | 1.5 | 1.5 | 2 |
| 22 | 509 | 2 | 2 | 2 | 2 | >2 | >2 | >2 | >2 | 2 |
| 23 | 510 | 2 | 2 | 2 | 2 | >2 | 1.5 | >2 | >2 | 2 |
| 24 | 512 | 2 | 1.5 | 1.5 | 2 | 1 | 1.5 | 1.5 | 2 | 2 |
| 25 | 513 | 1 | 0.5 | 0.5 | 1 | 1 | 1 | 2 | 2 | 2 |
| 26 | 514 | 2 | 2 | 1.5 | 1.5 | 1 | 1.5 | 1.5 | >2 | >2 |
| 27 | 515 | 2 | 1.5 | 2 | 2 | >2 | >2 | 1.5 | >2 | >2 |
| 28 | 516 | 0.5 | 0.5 | 1 | 0.2 | 0.5 | 0.5 | 0.2 | 0.2 | 0.1 |
| 29 | 518 | 1 | 1 | 1 | 1 | 1 | 1.5 | 2 | 1 | 1 |
| 30 | 519 | 2 | 2 | 1 | 1 | 1 | 1 | 1 | 2 | 1.5 |
| 31 | 520 | 2 | 1 | 2 | 2 | 2 | 2 | 2 | 2 | 2 |
| 32 | 521 | 1 | 1 | 2 | 1.5 | 1 | 1 | 1.5 | 2 | 1.5 |
| 33 | 525 | 1 | 0.5 | 1.5 | 1.5 | 1 | 1 | 1.5 | 2 | 1.5 |
| 34 | 526 | 2 | 3 | 2 | 3 | 1 | 1 | 1 | 1.5 | 1.5 |
| 35 | 527 | 1.5 | 2 | 2 | 2 | 1 | 1 | 1 | 2 | 1 |
| 36 | 528 | 1 | 1 | 1 | 1 | 1 | 1 | 1 | 1 | 1 |
| 37 | 529 | 1 | 1 | 1.5 | 1.5 | 1 | 1 | 1.5 | 1.5 | 1.5 |
| 38 | 534 | 1 | 1 | 2 | 2 | 2 | 2 | 1 | 2 | 2 |
| 39 | 554 | 1.5 | 1 | 1.5 | 1 | 1 | 2 | 2 | 2 | 2 |
| 40 | 555 | 2 | 2 | 2 | 2 | 2 | >2 | 1.5 | 2 | 2 |
| 41 | 6169 | 1 | 2 | 2 | 1 | 1 | 1 | 1 | 1 | 1 |
| 42 | 11828 | 2 | 2 | 2 | 2 | 2 | 2 | 2 | 2 | 2 |

Based on the results of Table 4, the two bacteriophages 50M and o50S were selected for further analysis. The phages were designated as “phiPA50M” and “phiPA50S,” respectively.

### Example 7

### DNA Sequences of C. acnes Bacteriophages phiPA50M and phiPA50S

The genomic DNA sequences of the phiPA50M and phiPA50S bacteriophage isolates was determined. Phage genomic DNA was extracted and purified with the purified phage lysates based on the procedure of Norgen Biotek Corp (ON, Canada). The purified DNA samples were sent to the Sequencing company (CD Genomics) for sequencing and analysis.

All contigs were perfectly assembled into 29,491 and 29,502 nucleotides for phage phiPA50M and phiPA50S, respectively. Both phages have 99% homology except for unmatched sequences about 12 nucleotides from phiPA50S. The phages were found to not harbor any antibiotic resistant genes or toxins. Blastn searches against other C. acnes phages showed maximum identities (99.88%) with Propionibacterium phage PHL082M03 (GenBank Accession no. NC_041955.1), Propionibacterium phage PHL082M04 (accession no. KJ578771.1). Based on the genomic sequences and homology search results were confirmed to be novel C. acnes phages

Putative endolysin genes from both phages phiPA50M and phiPA50S were analyzed with multiple alignment and found that both have 855 nucleotides (284 amino acids). It matched 99.65% with that of putative endolysin [Propionibacterium phage PHL082M00] (accession no. YP_009150025.1). Based on the genomic sequences and homology search results were confirmed to be novel endolysin derived from two C. acnes phages.

The sequence of the open reading frame (ORF) coding for the phage endolysin was analyzed with the Basic Local Alignment Search Tool (BLAST) against Conserved Domains Database (CDD) for identification of the amidase domain.

Bioinformatic analysis of the phage phiPA50S genome suggested that nucleotides 15095-15952 (855 bps) of ORF 19 encoded the 284 amino acids endolysin and that the protein belonging to the N-acetylmuramoyl-L-alanine amidase AmpD family (COG3023), with residues 13-173 belonging to the amidase 2 family (pfam01510). Residues 175-285 had a sequence identity of 100% with residues 175-285 of putative endolysin (Propionibacterium phage PHL082M00), corresponding to the cell wall binding domain of this enzyme, with no associated catalytic activity. A gene fragment encoding residues 1 to 174 of the putative phiPA50S phage endolysin is the predicted amidase domain.

This endolysin derived from the phage phiPA50S named as ‘LPA50S’. The acne phage lysin constructed consists of one catalytic domain (Amidase-2 or N-acetylmuramoyl-L-alanine amidase) and one cell wall-binding domain

### Example 8

### Recombinant Bacteriophage phiPA50S Endolysin

Based on the DNA sequences of phiPA50S, a pair of primers were designed to cover the full domain of the endolysin LPA50S. Other primer sets were also designed to cover the endolysin gene plus flanking N-terminal and C-terminal residues. The full endolysin gene was polymerase chain reaction (PCR) amplified with an appropriate primer sets and recombinant Taq DNA polymerase (ThermoFisher). A PCR product of the full endolysin representing 855-bp in size was confirmed by DNA gel electrophoresis. A PCR product of the full lysin plus flanking N-terminal and C-terminal residues representing 1015-bp in size was also confirmed by DNA gel electrophoresis.

Both PCR products were purified by PureLink PCR Purification kit (ThermoFisher) and self-ligated with a commercially available expression vector pBAD-TOPOR (Invitrogen) and transformed in Escherichia coli TOP10 chemically competent cells (Invitrogen). The recombinant E. coli transformants including the endolysin gene randomly selected were DNA extracted, cut by restriction enzymes, and confirmed by DNA gel electrophoresis. Additionally, using the primer sets (pBAD forward and reverse primers) that located inside the expression vector was amplified PCR products and confirmed the size by DNA gel electrophoresis.

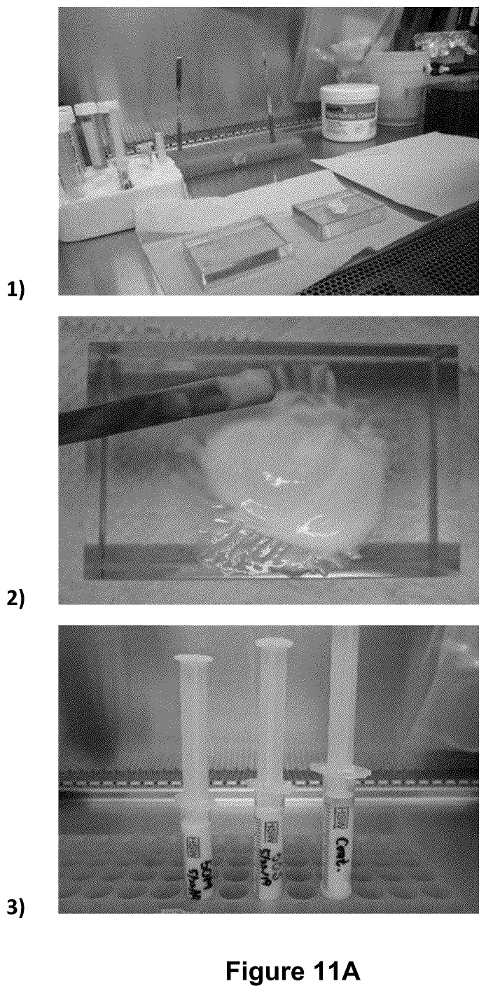

The potential E. coli transformants including the full endolysin gene, LPA50S was expressed in E. coli LMG194 strain under the araBAD promoter (www.invitrogen.com). In the presence of L-arabinose, the AraC gene product encoded on the pBAD-TOPOR plasmid positively regulates this araBAD promoter. Three E. coli clones (PLys35, PLys41 and PLys54) including (′. acnes-specific phage endolysin were expressed and analyzed by SDS-PAGE (FIG. 10A) and western blot analysis (FIG. 10B). Two bands of 38-kDa and 10-kDa from the PLys41 and PLys54 clones were detected by western blot analysis using by 6× His tag epitope antibody, confirming the expression of endolysin in E. coli expression system. It seems the 10-kDa protein band may be a truncated version of the full-length protein of (′. acnes-specific endolysin. The recombinant endolysin enzyme was confirmed its lytic activity against C. acnes strain by spot assay.

Endolysin protein expression levels were optimized by varying the concentration of L-arabinose (0.00002% to 0.2%) in order to determine the approximate amount of L-arabinose needed to ensure maximum expression of soluble protein. The recombinant endolysin protein with the C-terminal poly-histidine (6× His) tag was purified with use of a metal-chelating resin such as ProBond™ and Ni-NTA agarose (Invitrogen). The E. coli clone including the full endolysin gene, LPA50S may be cultured in a fermenter for large-scale cultivation and purification process.

### Example 9

### Topical Formulations of a Therapeutic Composition of Bacteriophage phiPA50M and phiPA50S

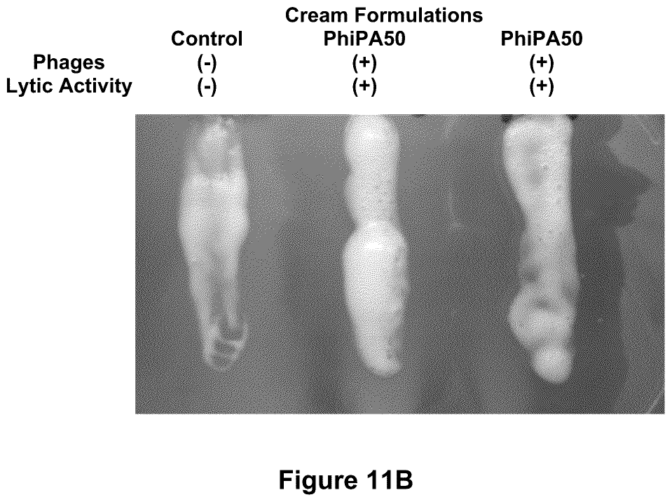

Two C. acnes-specific bacteriophages (phiPA50M and phiPA50S) were selected to prepare cream, hydrogel and poloxamer formulations. Three different types of base materials such as Cetomacrogol cream (a non-ionic base), Hydroxyethylcellulose (HEC, nonionic gelling agent, 1.5% w/v), and Poloxamer 407 (16% w/v) were prepared. One gram of each material on a sterile glass slab was mixed with each phage by gradual serial addition using a sterile stainless-steel blade to the cream to make a final concentration of 1.2×10⁸ PFU per gram for phiPA50M and 3.0×10⁶ PFU per gram for phiPA50S until all the fresh materials have been incorporated, and the phage is evenly dispersed throughout the base material. The phage formulation was added to a 3 ml syringe with the end wrapped with a parafilm for efficacy testing (FIG. 11A).

In order to assess the lytic capacity of the C. acnes phage formulations, C. acnes ATCC 6919 culture was plated on RCM agar plates. Each of the phage cream formulations (white color) was applied onto the top surface of the agar plate. The plates were incubated at 37° C. for 2 days under anaerobic condition to observe any lysis of the bacteria in the presence of the formulation by a clear zone. Both phage phiPA50M and phiPA50S cream formulations showed a lytic activity against (′. acnes 6919 (FIG. 11B). It indicates both phages formulated in cream have the therapeutic effect.

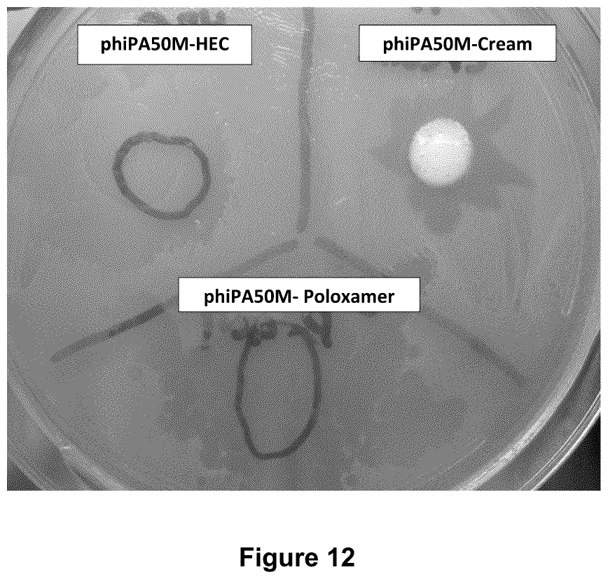

Base materials such as hydroxyethylcellulose (HEC), cetomacrogol cream (Cream), and poloxamer 407 (Poloxamer) were prepared with (′. acnes-specific bacteriophage (phiPA50M). Three types of C. acnes phage formulations (approximately 1.0×10⁸ PFU per gram) were assessed for their lytic activity against C. acnes ATCC 6919. Each of the phage formulations as marked with a black circle (due to no color) or white cream was applied onto the top surface, which C. acnes cultures was plated. Then the plates were incubated at 37° C. for 2 days under anaerobic condition to observe any lysis of the bacteria in the presence of the formulation by a clear zone. All three phiPA50M phage formulations showed the good activity against C. acnes 6919 (FIG. 12). The results indicate that the formulations combined with the C. acnes-specific phage have therapeutic effects.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-23. (canceled)

24. A composition to inhibit the growth of Cutibacterium acnes (C. acnes), the composition comprising:
at least one of:
bacteriophage Ø50S, whose genome comprises the sequence of SEQ ID NO:1 or a variant derived therefrom;
bacteriophage Ø50M, whose genome comprises the sequence of SEQ ID NO:2 or a variant derived therefrom;
a bacteriophage endolysin comprising the amino acid sequence of SEQ ID NO:4, SEQ ID NO:6, or a variant endolysin derived therefrom;
a bacteriophage holin protein comprising the amino acid sequence of SEQ ID NO:9, or a variant holin protein derived therefrom.

25. The composition of claim 24, wherein the composition comprises the bacteriophage endolysin and the bacteriophage holin.

26. The composition of claim 24, wherein the composition is a cream, gel, spray or liquid.

27. The pharmaceutical composition of claim 26, wherein the composition is a cream that comprises a hydrogel.

28. A method for treating or preventing acne, the method comprising the acts of:
providing a prophylactically or therapeutically effective amount of the composition of claim 1.

29. The method of claim 28, wherein the composition is provided using a bandage, medical device, or a medical implant.

30. The method of claim 28, wherein the composition is administered topically.

31. The method of claim 28, wherein the composition is provided to a subject having a C. acnes biofilm infection.

32. The method of claim 28, wherein the composition is applied to a wound or surgical site of a subject.

33. The method of claim 32, wherein the wound or surgical site is a shoulder wound or shoulder surgical site.

34. The method of claim 28, wherein the composition is administered as an aerosol.

35. The method of claim 29, wherein the bandage, medical device, or medical implant is applied to a wound or surgical site of a subject.

36. The method of claim 35, wherein the wound or surgical site is a shoulder wound or shoulder surgical site.


SEQUENCE LISTING (33 sequences)

SEQ ID NO: 1 - DNA, 29502 bp, Cutibacterium acnes (C. acnes) Bacteriophage Phi50S
agtgaaatacctcccttttttggttttgtctgtttgtcgactttttgtgttggtggtgagtgttgtgcagcctgagcttcctggtagtcgtgattggtgtggggagacgcgtcgttggtggcgtgtgtggggtgaggatagtcgcgcgccgtatgtgtctgatgaggagtggttgtttctcatggatgctgcggtgattcatgattgtgtgtggcgtgagggtcgcgcggatttggtggcttcgcttcgtgctcatgtgaaggcttttatgggcatgttggatcgttattcggttgatgtggcgtctggtggccggggtgggggttctgcggtggcgatgattgaccggtataggaagcgcaagggggcctgattaggtgtctggtgttgtgggttctcaggttcctcgtcatcgtgtggctgcggcgtattcggtgtctgctggcggtgatgctggggagttgggtcgtgcgtatgggttgacgcctgatccgtggcagcagcaggtgttggatgattggcttgctgttggtagcaatggcaggcttgcttcgggtgtgtgtggggtgtttgtgcctcgccagaatggcaagaacgcgatccttgaggttgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgggcgacgaatggtcaggaggctattgtgttgcatcacccggattgtgccacgtttgagaagaagtgtggctgtccgggttggggttcggttgagtttgtggcccgttctcggggttcggctcgcgggtttacggttgatgatttggtgtgtgatgaggctcaggagttgtcggatgagcagttggaggcgttgcttcctacagtaagcgctgccccgtctggtgatccgcagcagattttccttggcacgccgcctgggccgttggcggatggttcggttgtgttgcgtttgcgtgggcaggctttgtcgggtggtaaaaggtttgcgtggacggagttttcgattcctgacgagtctgatccggatgatgtgtcacgccagtggcggaagcttgcgggggatacgaatccggcgttgggtcgccgcctgaatttcgggaccgtaagcgatgagcatgagtcgatgtctgctgccgggtttgctcgggagcggcttggatggtgggatcgtggccagtctgctgcgtctgtgattccggcggataagtgggctcagtctgctgtggatgaggcgagtctggttggcgggaaagtgtttggtgtctcgttttctcgttcgggggatcgtgtcgctttggcgggtgccggccggactgatgctggggttcatgttgaggttattgatgggctgtcgggaacgattgttgatggtgtgggccggttggcggactggttggcggttcgttggggtgatactggccggatcatggttgccgggtctggtgcggtgttgttgcagaaggcgttgacggatcgtggtgttccgggccgtggcgtggtggttgccgatactggcatctatgtggaggcgtgtcaagccttcctggaaggtgtaaggtctgggaatgtttctcatcctcgtgctgattcgaggcgtgacatgttggatattgctgtgaggtcggcggttcagaaaaagaaaggctctgcgtggggttggggttcctcgtttaaggatggttctgaggttcctttggaggctgtgtctttggcgtttttgggggctaaacgtgttcgtcgtggccgtcgggatcgtagtggtaggaagcgggtgtctgtggtatgaacgtggacgagttggctctgattgagggcatgtacgatcgtatccaaaggttgtcttcgtggcattgtcgtattgagggctactatgagggttctagccgggtgcgtgatttgggggtggctattcctccggagttgcagcgtgtgcagactgtggtgtcgtggcctggtatagctgtggatgctttggaggagcgtctggattggctgggctggactaatggtgacggctacggcctggatggtgtgtatgctgcgaatcggcttgctacggcgtcgtgtgatgtgcatttggatgcgctgatttttggtttgtcgtttgtggctgttattccccaggatgatgggtcggtgttggttcgtccgcagtcaccaaagaattgtactggccggttttcggctgacggttctcgtttggatgcgggtttggtggtgcagcagacgtgtgatcctgaggttgttgaggcggagttgttgctgcctgatgtgattgttcaggtggagcggcgtgggtctcgtgagtgggttgagacgggccgtataccgaatgtgttgggtgctgttccgttggtgcctgttgtgaatcgtcgccgtacgtcgaggattgatggccgttcggagatcactcggtcgattcgtgcttacacggatgaggctgttcgcacactgttggggcagtctgtgaatcgtgacttctatgcgtatcctcagcgttgggtgactggcgtgagcgcggatgagttttcgcaacctggctgggtgttgtcgatggcttctgtgtgggctgtggataaggatgatgatggtgacaccccgaatgtggggtcgtttcctgtgaattctcctacaccgtattcggatcagatgcgtttgttggcgcagttgactgcgggtgaggcggctgttccggaacgctatttcgggtttatcacgtctaacccgccttctggggaggctttggctgcggaggagtctcggcttgtgaagcgtgctgaacgcaggcagacgtcgtttggtcagggttggttgtcggttggtttcctggctgcccgggcgttggattcgagtgttgatgaggccgcgttttttggtgatgttggtttgcgttggcgtgatgcttcgacgcctacccgggcggctacggctgatgctgtgacgaagcttgttggtgccggtattttgcctgctgattctcgtacggtgttggagatgttgggtttggatgatgtgcaggttgaggctgtgatgcggcatcgtgccgagtcttcggatccgttggcggcgctggctggggctatttctcgtcaaactaacgaggtttgataggcgatggcttcgggggctatgtcgaggcttgctgcgactgagtatcagcgtgaggctgtcaggtttgctgggaagtatgcgggctattattctgagcttggtcgtttgtggcgtgccggcaggatgagtgacacgcagtatgttcgtttgtgtgtggagttggagcgtgccggccatgatggttcggcatcgttggctgccaggtttgtgtcggattttcgccggttgaatggtgtggatccgggtttgattgtgtatgacgagtttgatgctgcggcggctttggctaggtcgttttcgactatgaagattcttgagagtgacccggatagggcgaatgacacgattgatgcgatggctgcgggttttgatcgggctgtcatgaatgctggccgtgacacggttgagtggtctgcgggtgcgcagggtaggtcgtggcgcagggtgactgatggtgatccgtgcgcgttttgtgccatgttggctacgaggtcggattatacgactaaggaaagggcgcttactactggtcatacgcggcgtcataagcgtgccggtaggcgtccgtttggttcgaagtatcatgatcattgtggttgtacggtggttgaggttgttggcccttgggaaccaaatagggctgatgccgagtctcagaggacgtatgagaaggcccgtgagtgggttgatgatcatgggttgcagcagtcgcctggcaatattttgaaggctatgcgtactgttggcgacatgagatgatggtttccggttgtgtgccgccggttatcggtgcacagggttgtctcccgcacgggggtcaacaatgttgtgttgttttccgcaaggagtatagggttaggctatggccgatcaaaaagttgaagaacagaatgttgacaatgatgctgttgagcccggaaagggtggagacgttgttgatgttgtgaaggatgggcaggcttccggcgatgatcatgccggtgatgtttccgtgaaggaggagtcttcttctggcacggattggaaggctgaggcccgtaagtgggagtctcgtgctaaaagtaatttcgccgagttggagaagcttcgtacatcgagtgacgattctggatctactattgatgagcttcgccgcaagaatgaggaactcgaagacaggatcaacgggtttgttcttgagggtgtgaagcgcgaggtggcttcagagtatggtttgtccagtgatgcggtcgctttcttgcacggtggcgatcgtgaagcgctggtggagtctgctaaggctttgaagggtttgatcgaccagagtggtggtggcgcgggtgtgcgccgtcttgcggggagtgcccccgttgatgatgttaaacgacgtgagggtgtcgcgtttgtggatgctcttgtcaataattctaggagatgatttctgatggctgacgattttctttctgcagggaagcttgagcttcctggttctatgattggtgcggttcgtgaccgtgctatcgattctggtgttttggcgaaactgtcgccggagcagccgactatttttggcccggtgaagggtgccgtgtttagtggtgttcctcgcgctaagattgttggtgagggcgaggttaagccttccgcttcggttgatgtttcggcgtttactgcgcagcctatcaaggttgtgactcagcagcgtgtctcggacgagtttatgtgggctgatgctgattaccgtctgggtgttttgcaggatctgatttctcccgctcttggtgcttctattggtcgcgccgtggatctgattgctttccatggtgttgatccggctacgggtaagcctgctgcggctgtcaaggtgtcgctggataagacgaagaagacggttgatgccaccgattccgctacgactgatcttgttaaggctgtcggcttgattgcgggtgccggtttgcaggttcctaacggggtcgccctggatccggcgttctcgtttgctctgtctaccgaggtgtatccgaaggggtctccgcttgccggtcagcctatgtatcctgccgccgggtttgccggtttggataattggcgtgggctgaatgttggtgcttcttcgactgtttcgggtgccccggagatgtcgcctgcctctggtgttaaggctattgttggtgatttctcgcgtgttcattggggtttccagcgcaatttcccgatcgagctgatcgagtatggtgatccggatcagactgggcgtgacctgaagggccataatgaggttatggttcgcgccgaggctgtgctgtatgtggctatcgagtcgcttgattcgtttgctgttgtgaaggagaaggctgcaccgactcctcctccggccccgaagcctgagcctaatcctccgtctggtaactgatacaagataagcgaatgtgtactatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgcagggctttgttgcgctggaatgataccggggtttcgggtcaggtgcagtatgagtctgcgggtcctttcgctcagactacacggtctaatactcccacgaatttgttgtggccttctgagattgccgcgttgaagaagctgtgtgagggtgatggtggggctggtaaagcgttcactatcacacccactattaatggtcgatatgtgcattctgaggtgtgttccacggtgtggggtgagggttgctcgtgcggatctgatattaacggctacgctggccctttgtgggagatatgatatgaccagttttccttatggtgaaacggttgtgatgcttcagccgactgttcgtgtcgatgatcttggcgacaaggtggaagactggtctaagcctgtcgagactgtgtaccataacgtggccgtttatgcttccgtttcgcaggaggatgaggcggctgggcgtgactctgactatgagcattggtcgatgctttttaagcagtctgttgtgggtgccggttatcgttgccggtggcgtattcggggtgttgtgtgggaggctgacgggtctcctatcgtgtggcatcacccgatgtctggctgggatgcgggcacgcagatcaatgtgaagcgtaagaagggctgatagattgtggctcaggatgtgaatgtgaagctgaacttgccgggtattcgtgaggtgttgaagtcttttggggtgcaggctatgttggctgagcgtggcgagcgtgtcaagcgtgcagcctcggcgaatgtgggcggtaacgctttcgataaggcccagtatcgtgcagggttatcatcggaggtgcaggttcaccgtgttgaggctgtggcccgtattggcaccacatataagggtgggaagcgtattgaggcgaagcatggcacgctggctcgttcgattggggctgcgtcgtgattgtttacggtgaccccaggaagtgggctaaacgcgtgctcaaggatgatggctggctgtctgggataccgtgtacagggacggtgcccgatgattttacgggtgatctgatttggttggctcttgatggtggcccgcagttgcatgttcgtgagcgtgtttttttgcgggtgaatgtgttttctgatatgccggatcgtgctatgtctttggctcgtcgtgttgaggctgtgctggctgatggtgtggacggtgaccctgtggtgtactgtaggcgttctactggacctgatttgctggttgatggtgcacgttttgatgtgtattcgctttttgagcttatatgtaggcctgcggagtctgaataagcttattgtttttgttttaatgtaattgtttgatatttaatgggggttatgatggctgcaacacgtaaagcgtctaatgttcgctctgctgttacgggtgacgtctatattggtaaagctcatgccggtgacactattgatggtgtgaagacggttcctgacggtcttaccgctttagggtacctgtcggatgacgggtttaagattaagcctgagcgtaaaacggatgatttgaaggcttggcagaatgcggatgttgttcgcactgtggctacagagtcgtctatcgagatctctttccagctgatcgagtctaagaaggaggttatcgaactgttttggcagtcgaaggttactgccggagccgattctggttcgttcgatatttctcctggtgccaccactggcgtgcacgccctgttgatggatattattgatggcgatcaggttattcgctactatttccctgaggtcgagttgatcgatcgtgacgagatcaagggcaagaatggcgaggtgtatgggtatggtgtgacgttgaaggcgtatcctgcccagattaataagaagggtgatgcggtgtctggtcgggggtggatgacggctttaaaagctgatactcccccggttcctccttctccgaagccggagcctcctaagcctgagccggatccgaatccgccgtctaataactgatacacatagtttgagggattgttgatagatgagtgacacaggttacacgttgaagattggtgaccgtagctgggtgttggcggatgcggaggaaacagcgcaggctgttcctgcccgcgtttttcgccgtgccgccaggattgcccagtctggggagtctgctgatttcgcccaggttgaggtgatgttttccatgttggaggctgccgccccagaggatgctgtggaggccttggaggggcttcctatggttcgtgtggccgagattttccgtgagtggatggaatataagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccacctgaatggttggcagcgcccgtttgagtggtgcgagtgggctgtgctggacatgctggatcattacaggtctgctaatagtgaggggcagccggagcctgtggcgaggcctacggatgagcgtagggcccggtttacgtctgggcaggtggacgatattttggcgcgtgttcgtgccggtggcggggtgtctcgcgagattaatattatggggtgaatagtgtatgtctggtgagattgcttccgcatatgtgtcgttgtatacgaagatgcctggccttaaaagtgatgttggtaaacagttgtcgggtgtgatgcctgctgagggtcagcgttcgggtagcttgtttgctggcgggatgaagttggcgcttggtggtgcggcgatgatgggtgccatcaatgttgctaagaagggcctcaagtctatctatgatgtgactattggtggcggtatagctagggcgatggctattgatgaggctcaggctaaactgactggtttgggtcatacgtcgtctgacacgtcttcgattatgaattcggctattgaggctgtgactggtacgtcgtatgcgttgggtgatgcggcgtctacggctgcggcgttgtctgcttcgggtgtgcagtctggcgggcagatgacggatgtgttgaagactatcgccgatgtgtcttatatttcgggtaagtcgtttcaggatacgggcgctatttttacgtctgtgatggctcgcggtaagttgcagggcgatgacatgttgcagcttacgatggcgggtgttcctgtgctgtctctgttggctaggcagacgggtaaaacgtctgctgaggtgtcgcagatggtgtcgaaggggcagattgattttaacacgtttgcggctgcgatgaagcttggcatgggtggtgctgcgcaggcgtctggtaagacgtttgagggcgctatgaagaatgttaagagcgccctgggctatcttggtgctacggctatggccccgtttttgaacggcctgcggcagatttttgttgcgttgaatccggttattaagtctatcacggagtctgtgaagcctttgtttgcgtcggtggatcaggggattcagcggatgatgccgtctattttggcgtggattaaccggatgccggctatgatcacaagaatgaatgcacagatgcgcgccaaggtggagcagttgaagggcatttttgcgagaatgcatttgcctgtccctaaagtgaatttggctgccatgtttgctggcggcaccgcagtgttcggtcttgtggctgccggtgtggggaagcttgttgcggggtttgccccgctggcggtgtcgttgaagaatctactgccgtcgtttggtgctttgaagggtgccgctggggggcttggcggcgtgtttcgcgccctgggtggccctgtcggtattgtgatcggcttgtttgcggcaatgtttgctacgaacgcccagttccgtgccgcggtgatgcagcttgtggctgtggttggccaggctttggggcagattatggccgctgtgcagcctgtgtttggtctaattgctggtttggtggcacggttggctcccgtttttggccagattatcggtatggttgccgggttggctgcgcagcttatgcctgtgattagtatgcttgtcgcccggctggttcctgttatcacgcagattattggtgcggtgacgcaggttgctgcaatgttgttgccggcgttgatgccggtgttgcaggctgttgttgctgtgatacggcaggttgttggcgtggtcatgcagttggtgcctgttttgatgcctgtgattcagcagattttgggtgctgtcatgtctgtgctgccacctattattggtcttatccggtcgttgatgcctgtgattgcggcggttatgcgtgtggtggttcaggttgttgcggttgtgatacaggtggtggcccgtattcttgctgttgtggctccgatggtggcggccgtggtcgggtttgttgcccgtattgttggtgctgtcgtgtcggctgtggcccgtgttattgccgctgttgcccgtgtcatctcgtgggttgttgcccattttgtgtctggtttggcgcgtatgggttcggtggttcaggctggctggaatcatattagggcgtttacgtctgcgtttattaacggtttcaagtcggtgatttctggcggcgtgaacgcggttgtggggttttttgcccggcttggttcttcggttgcctcccatgtgaggtctggttttaacgcggctcgtggcgctgtttcttctgcgatgaatgctatccggagtgttgtgtcttcggtggcgtctgctgttggcgggtttttcagttcgatggcgtctagggttcgtagtggtgctgtgcgcgggtttaatggtgcccgtagtgcggcttcttctgctatgcacgctatgggctcggctgtgtctagtggtgtgcatagtgtgatagggtttttccggaatctgcccagcaatattaggggcgctttgggtagtatggggtccctgttgatgtctgctggccgtgacgtggtggccggtttgggtaatggtattaagaatgctatgagtggcctgttggatacggtgcgtaatatgggttctcaggttgctaatgcggcgaagtcggtgttgggtattcattccccgtctcgggtgtttcgtgacgaggttggccggcaggttgttgctggtttggctgagggtattactggtaatgctggtttggcgttggatgcgatgtcgggtgttgcttcgcagcttccggatgctgtggatgcccggtttggtgtgcgatcgtctgtgggctcgtttaccccgtacgaccggtatcggcgtgctcagggtgagagtgttgtggtgaatgtgaatggcccgacgtatggtgatccggccgagtttgcgaagcggattgagcggcagcagcgtgacgctttgaacgcgttggcttacatgtgagtaaaggggtatgcatgtttattcctgacccgtctgatcgttctggtttgactgtgacatggtcgatggatccgcggtttggcgacgagcgtgtgcttcatttgacggattatacggggtcgtctcctgtcatgttgttgaatgattcgttgcgcggtttgggtgttcctgaggtggagcatttttctcaaacgcatgttggggtgcacggttcggagtggcgcgggtttaatgtgaagcctcgcgaggtgacgctgccggtgttggtgtcgggtgttgacccggatccggtgggcgggtttcgtgacggttttttgaaagcctatgacgagttgtggtctgcgtttcctcctggcgaggagggggagttgtctgtgcagactcctgccggtcgtgagcgtgtgttgaagtgccggtttgattcggtggatgacacgtttacggttgatccggtgaatcgcggctatgcccgctatgtgattcatttgacggcctatgacccgttttggtatggggatgagcagaagtttcgttttagtaacgctaagttgcaggattggctgggtggcggccctgtcggcaaggatggcacggcgtttcctgtggtgttaacaccgggtgtgggctcgggttgggataatctgtctaataagggtgatgtgcctgcgtggcctgtgattcgtgttgagggcccgttggagtcgtggtctgtgcagattgatggtttgcgtgtgtcttcggattatcctgtcgaggagtatgattggatcactattgatacggatcctcgccagcagtcggcgttgttgaacgggtttgaggatgtgatggatcgtttgacagagtgggagtttgcgcctatcccgcctggcggttcgaagagtgtgaatattgagatggttggtttgggtgccattgttgtgtcggtgcagtacaggtttttgagggcttggtgaatggttgatggctggtcttgttccgcatgtaacgttgtttacgccggattatcgccgtgtggcgcctatcaatttttttgagtcgttgaaactgtcgttgaagtggaatggtttgtccactttggagttggtggtgtcgggtgatcattctaggcttgacgggttgactaggccgggtgcacggctggttgttgattatggtggtggccagattttttctgggcctgtgcgtcgggttcatggtgtgggtccttggcagtcttcgcgggtgactatcacgtgtgaggatgatatccgcatgttgtggcgtatgctgttgtggcctgtgccttatcgttccagcatcattgggatggagtggcgtgccaatagggattatgcccactattcgggtgcggcggagtcggtcgctaagaaggcgttgagggataattcttggcgttttccgcctgatatatttatggtgaatgataagagtcgtggccgctatattaaggattttcaggcgcggtttcacgtgtttgccgataagttgttgccggtgttgtcgtgggctcggatgactgtcacggtgaaacagtttgagaatgtgaagcaggatcagcgtggtttactgtttgattgtgtgcctgctgtgacgcgtgagcatgtgttgactgccgagtctgggtctattgtgtcgtgggagtatgtgcgtgacgccccgaaggctacggcggtggttgtgggtggccgcggcgaaggcaaagatcgtctgttttgtgaggattttgacgcgttggccgaggatgagtggtttgatcgtgtcgaggtgtttaaggatgcccgtaacacggattctgagcatgtgcatctcattgatgaggcggagcgggtgttgtccgagtcgggggccacgtcggggtttaagatcgagttggctgagtcggatgtgttgcggtttgggccaggcaatctgatgccgggtgatttgatctatgtggatgtgggctcggggcctattgcggagattgttcggcagattgatgtggagtgtgtatcgcctggtgacgggtggacgaaggtgactcctattgcgggggattatgaggataatccgtcggccctgttggctcgccgtgtggctgatttggctgcgggtgtgcgggatttgcaaaaattttagaaaagaatcgggggtttgttgtgggtattgtgtgcaagggttttgatggtgtgttgaccgagtatgattgggctcaaatgtctggtctgatgggtaatatgccgtctgtgaagggcccggacgattttcgtgtcggcactactgttcagggtgccacagtgttgtgtgaggtgttgccggggcaggcttgggctcacggggtgatgtgcacgtcgaatagtgttgagacggtgacagggcagctgcctggtcctggcgagacccgatacgactatgttgtcctgtctcgggattgggagcagaatacggccaagttggagattgttcctggggggcgtgcggagcgtgccagggatgtgttgagggctgagcctggcgtgtaccatcagcagttgttggcgactttggtgttgtcgtctaacgggttgcagcagcagttggataggcgtgctgtggcggcccgtgtggcgtttggggagtctgctgcgtgtgaccctacccctgtggagggtgaccgggtgatggtgccttcgggggctgtgtgggctaaccatgctaacgagtggatgctgttgtccccgcgtatcgagacgggttcgaagcagatccagtttggcgggtctgctgtgtatgcttacacgatcccgtttggccgcccgtttggtagtgcgcctgtggtggtggcgtctatggcgacggcggctggtggtacgcagcagattgatgtgaaagcctacaatgttactagcaaggattttcagttggcgtttatcacgaataatggttctaagccttctggtgtgcctgcggttgctaactggattgctgtcggcgtgtaatgtacggcttgcgggtatgtgacgtgttgtggttgttgtagtggtagtgggctgtagtgtcatggcttacacccacactcgtagcctctctttgtaccgctatcgctactgttcttggttcgattcaggcggctacgtacaggtcgaagaagaggctgaggcagttgtctgcgcaggttgatgcgatggaagaatacacgtggaatattcgccatactgttcaccgctataatgcgaatctgccggagaatgttgagccggtaaagatgcctgatttgcccgagtttttgaaggatactgttgatggtggtggggggtgaattgtgagggagttggaggaagagaagcggcagcgccgctcgtttgagaaggcttccatgatactgttgttcctgtcgcttgtactactggcggtggtcgccgggggtgctttacgtttcggtgctgtggcttcccagcgggattcggagcaggctaaagcccagtctaatggtacagccgctaaagggttggctagccgtgtgcggcaggtgtgtgcctctggcgggcaggagtcggtgcggcttcaccagtctggcttgtgtgtggatgctcagcgtgttgagcgtagcgtgcagggtgttcctggtcctgccggtgtgcgtggcccgcaagggcctgcaggggttgacggccgggatggtgttgatggttcggctgggctggttggccctgttggcccgcagggttcccctggtttgaatggtgtgaagggtcctgacgggctgcccggtgctaacggttcggatggccgtgatggtgttgccggtgtgaacggggctgatggcaaggatggtgtgccaggtagggatggcgctgatggggctgatggtggccgcggccctgttgggcctcaggggccgcagggttctgccggtgccgatggcaaggacggtaaggatggcgctgatgggcgctcggtgatatcggtgtactgttccgggggccgcctggttgtgaaatatagtgacggtacggcctctaccatagcgggttcggtggcctgtgagggtgtgaaaccatcaccggtggttaccgtatcatcccacaagtaaaagaggaagtgtgttactagtgttgatagtagtgtttgggggtggcgtgttgtgagatacattccagcggcgcatcactcggccggatcaaataagccggtgaaccgtgttgtgattcacgcgacatgcccggatgtggggtttccgtccgcttcccgtaaggggcgggcggtgtctacagcaaactattttgcttccccatcgtcgggtggttctgcccattatgtgtgtgatgttggggagacggtgcagtgcttgtcggagtctacgattggttggcatgccccgccgaatccgcattctttgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtgatggtggtttgttgtgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcataagtgaatatagatgtgtgccccagcggtgctgccacgatcgtgtggtggttgccgctggggcactatttttgtgtctatagtattctatgattcgttgctgtcgatggtttcttcgatcagctggtccaggtggatgcaggcggagatagtatcgttggcctggtgcagaacgttctggccgataacatttttataattgtcgcggtggcagatgatagactgcatgatatcgtcggcttccgcttgcaatagtttggcctggtatgcgattccggcgagccaatctatggcttcctggcttgcccgtgtgtcgtctggaatgccacgggtgttgctgttgtttgggtatcctgcactgtcgcagtcccacaagatttcgctgcactcgtctagcgtgtcctgatcgatagcgagatcgtcgaggctgacttctttgacggtaaggttcacgttgtcgagtgagatgggtacacggtactggttttcgacaccgtcaacaatgttttccaattgctgcatgttggtgggctgttgttggatgattcggtgtactactgttttgagggcggtgtaggggatattgtgtgtgttgtccatcgtgttatgccattccttcgttatcgtctggcatgtagtatgtgctgtttgcgtactcggttaacgtcatcagtgtttggtctgcccactgtttcacggtttgccgggtgactccgagtcgttgggctgccaccgaataggtttggtcatacccgtagacttcccggaacgctgccaacctagctagccgtttcctttgtttggatggctggcaggtgagggtgtagtcgtcgatggctagttgtagatcgatcatggtgacaatgttgttgccgtggtgttgtggcgcggtgggtggtggtggcattcctggttcaacactcggtttccatggtccgccgttccagatccattgcgcggcttggataatatcggctgtggtgtaggtccggttcatgtgtcatcccctgaataggttgtcgaggttgtctgggttgctggtgtcgaatcgtccgacgcagtggcagtagtcgtacatgagtttgatgatgtgttggtggtctcccaaataggtgtttccgctgatgctgtaggtggctgtgccgtctttactaatagtgtatttggcggtgatggtttcgggtgtttcggtgttggtgatgatggcggtggtggtggcgcctactgtttgtagcctggtggtttgggtgccgtcgtcgaggatggtggtgaccattatgattctccttagttgctggtttggttgtcggctagatgaataatatcggataaaggtttcggctggtctaaatgttgtgtggttttgttggctagccgtttggctaccctgtagcacattttggtgtagtgtttgttgtctaggttgtggtattgttcccgcaccgcaatatatagtagggagtcttggtagaggtcgtctgcgctgattgcggggtagtgtgtaaccactttggtgcatgcccggttgagtgtgcgtagatgatggtctgtggcccatccccacgatgcggtggtggctaggtctgcttttgttggtcgcctgctcatggcactattacctggctatctggtagttgtttggtgttttgttgttgatagtgtagcacacgagtccggggtttccggtggtgcccgtcttgtgccggaaccatgtggattcgccttccatggatgggcattggatgaaggtgcgtgtgccttgctcagagatttctaggtggtgccggtgcccggccatcaggatgtgggatgtggtgccgttgtggaattcttggccgcgccaccattcgtagtgtttgccggtgcgccattggtgcccgtgggcgtgcaggatttgtgtgcctgccacatcgacggtggtggtcatttcgtctcggctggggaagtggaagtgaaggttggggtattggttgttgagctggtaggcttctgcgatggcgcggcagcagtccacgtcgaaggagtcgtcgtaggtggtgactccttttccgaagcgtacggcttctccgtggttgccggggatggatgtgatggtgacgttggcgcagtggtcgaattggtggatgagttgcatcatggccatgcgggtgagccggatttgttccgtcaggggtgtttgtgtgcgccaggcgttgttgcctccttgtgacacgtatccttcgatcatgtcgccgaggaaggcgatgtggactcgttgcggtttgcctgcttgctgccagtagtgttttgcgacgatgagggtgtgtaggtagttgtcggcgaagtgtgctgtttctccgccggggatgcctttgccgatttggaagtctcctgccccgatgatgaaggctgcagtgctgtagtcggtgtgggtgtcttgttcgggttttggtggctgccattcggctagtttatcgacgagttcgtctatagggtaggggttggttacgggttggtggtcgatgattttttgtatggatcggcctgtttctccgttggggagtgtccattcggagatgcgtgtgcggcgcacggtgccgttggctagattgtcgtcgatggtgtcgatggcgttgtcgtggttggctagctgtgtgaggagccggtctatgttgtctatcactgggtatcatcctcttcctcgtgtgtggtggtggcttgtttgcggcgatggtctttgataacggtggcggagatggggtatccggcttcagtgagcatttgggctagctgtgtggcggggatggacctgtcggcgaggacgtctgcggctttgttgccgtagcgttggatgagggtttcagttttggttgccatgatgtcctatcggttgtgtggtgggttgccatcctgtgcggcagtcgccgtcgtgtcctggtttgcgtgtgcaccatgagacttcgccggcattgtggatgatggcacggccgcatatgacgtcatgtaggtgttcgggaaacttatcgttgttgttgtccccgtacatgtcgatcaagtgttgggttttagtaaccatcatgtctcctatgtgtgaaagagtgtgcaaatactatgctggtgtcatggatgtttatgcgggtatggttttcatcaccttgctgaacgttacttggttactgtacatcatctgggtgatttcctgatccgttttgtcggggtgctgttttcgcaggtttgcccattggcaggcgttgtcggtttcctgctgtaaacgtgtcaggtgttgttcgttgatgatgtgtttccacattgtccacgagacgtcgagtcgtttgagcatgtcgatggctggcacgttgaaggagttgaggaagagtatttcctccgtgtagtagtctttttcgtattggtcccatccgcttcggtgcctgttgggctggtttttagggtaggcttcccggcatactttgtgcaaacgcttggccatgtctttgggtagcctaatgtcggggttggcgcggatcatggatcgcatcccatcataggtggtgccccaggtgtgcatgatgtggagtgggtcttcaccatcagcccatttttctgcacagatggcgaggcggatacgcctccgggcggctttgctagtgtcgcggcggccggggatggggcatgtgtcgagggggtccatgatgctttttatgcctttctttgtttggtttgcttgtgtggttttattgtagcactgtgtctagtgcttgtgtcaaccctgtttttccggcctgcaggtaggtgtctgtgacatcgcccagggtgaggggcacgcgggtggcttgggggagtgccgtctggagggtttgggccatctggtctcctgctttgtcggggtctgaccagatgtagatgtggtcgtagccttcgaagaatttggtccaaaagttttgccacgaggttgcgccgggtagggctacggccggccatccgcattgttcgaggatcatggagtcgaattcgccttcgcaaatgtgcatttcggctgccgggttggccatggcggccatgttgtagatggagcctgtgtcccctgccggggttagatatttggggtggttgtgggttttgcaatcatgctggagtgagcagcggaaacgcatttttcgtatttcggctgggccgtcccagacggggtacatgtaggggatggtgatgcactggttgtagtcttcgtggcctgggatggggtcattgtcgatgtatccaaggtggtggtagcgggctgtttcttcgctgatgcctcttgccgagagcaggtcgagtatgttttcgaggtgggtttcgtagcgggctgaggctttctggattcggcggcgttccgcaatgttgtagggttgtagtgagtcgtacattcgggttttctttctctaattgttgtttcagtttggcgaggcctcctccgatgccgcatgtgtggcagtaccagacgcccttgtcgaggttgatgctcatggagggctggtggtcgtcgtggaacgggcagaggatgtgttgctcgttcctggatggattgtaccgtatctggtaggtgttgaggaggcggcaggtgtcagaggtgtgggaggagctcgttgagggttgataccacataggcttcgctccatggcttgttgcgctgtttcatcactacgagtccgatggtggaattgttttctcggtttcggtgggtttcgtagttgcgtgcctcccggctggcttgtttcacgaattcggcgaggtgtgcctgtcctgctttggcttcgatcacataggttttgtggccggttgtgaggatgaggtcgccttcatcctctttaccgttgaggtggaggcgttctatatcatagccggtgtcgcgtagctggtggaggagtcgtgtttcccattcggctccggcccggcggttgcgtgcctgttgtgttgacatgatagtcctttgtggtgttcggtcatgttccatggctgtttttcggcgagtggcccgaagaatgtgtattcggggtaggctcgcagtcgttcgtatcgggtgccgtctgggctggatttgcctgtgcgctgtttgagtacggcgatgcgtgcctctgccggtatcgtgagcccgttgccgttatcttcgccaccatacagggagactcccaatatgagttgtggtttttcggagaggccgtttttgatttcccgcctagccggggggtgttcgatgtcggtgccggttttgtcggttgcgtggtgtgtgacaataatggtggagccagtatccctacccaatgctgtgatccattgcatggcttcttgctgtgcctggtagtcggattcgcagtcttggatgtccatcaggttgtcgatcacaatgagtggtgggaaggtgttccacatttccatgtaggcttgcagttccatggtgatgtctgtccatgtgatgggtgactggaatgagaatgtgatgtgttggccgtggtggatgctgtctcgatagtattctggcccgtagtcgtcgatgttttgttgtatctgggcggtggtgtgttgagtgttgagtgagatgattcgtgtggaggcctcccagggtgtcatgtcccctgatatgtagagtgcgggctggttgagcatcgctgtgatgaacatggctagcccggatttttggctgccggaccgccccgcgatcatgacgagatcccctttgtgaatgtgcatgtccaggttgcggtagaggggttctagttgtggtatgcggggcagttcggctgcggtttgggaggctctctcgaaggatctttggagagagagcatcggagccttaatctatctgtctatcggttggatgatgttttggtggtcagatggagtcgatgtcgatgtcagcatcagcaggggctgtggtgtcgtctagctggccgttatcgcgtttgtctacgtattcggcaaccttatcgtagatggcgtcgtctaatggtttgagcacgaccgcgttgaacccgtttttggtgcgcacggtggcgagtttgaatgcctgttcttcgccaaggtaggcttctaggtcgcggatcatggagtgtgggcggtcgttgttgccgcgggctttctcgataatggcgttggggatggtttctggggtgccgttgttgagatcgtctagggtgtggaagattgtgacatcagcgtagatgcgatcggcgacctgtccaccgtagccttcggtgttgtgctggacgtcgcggattttgaaggcgatggcggtggcgtcctggtttcgggaggggttgaagaaggtgctgttgctgttgttgcggtagtttgcgagtcccattgttgtttcctttactgttttgttggtttgtttgttggttttatcgggtgaggctgtttcgtttgctgcggaaagcctcggaaacgtcactgttactggtgatgatctttttgtactgtttgaggaggtcggctagctgtgctttgcttgttgcattgttgattttgtcgatgatggtgttgtttccttctgaggcgatgttgtctacgtagtctttggcggcctggttgtatcggtcttggaggattatggatgctgtggcgatcagtgttgccaggtcccagttccttgccgcggagctgtttttgagtccgccgaggaggtcgatgatagtcttctttacctggtcggcggtgtctccgcggatgacggtccatggggcggcgtagtcgcctccgtatttgagggtgacggtgaatcggtcgtcgtctgtgttgtcggtcactggtgctccttgtcttcttttgttggggctgtgatggtggtttctatggggtacctgtaggcgtctttcccgttgacagcccagcaggcgtccttgacggggcagcctttacagagtgctgtgacgtggggtacgaagatgccttggctgattcctttcattgcttgactgtacatggatgatacatgccggtaggtgttgttgtcaaggtcatagagttcggttgctgtgccttgtgtcggagactggtcgtcgttgcggctggtggctggcgtccaaaacatgcctttcgtcacatcgttgccgtgttggttgagcatgtaccggtatgtgtgcagctgcatactgtcggcgggtaggcgtccggttttgaggtccaaaatgaaggtttcaccggtgtcagtgtcggtgaaaacgcggtcgatgtagccgacaatagtggtgccatcctggagggtggtttctaccgggtattcgatgcccggctggccgtcaataacagcggtgatgtattctgggtggttgcgcctccatgttttccaccggttgacgaaggtggggccgtatatcatccaccaattgtagtcttttttgtgtggcccgcccgattcgcacatgtttttgcatattcggccggagggtttgatgtttgtgccttctgatttggcgagggcgacttgggtgtcgaaaatgtttttgaaggatgagagtttgtctggcagtgcagggtattcggcgggattgtacaggtgtaggtcgtattgttcggtgatgtggtgtatggcgcttccggcgatggtggcgtaccaggtgtggtgttgggtgtggtagccgtgggataggcgccatttttctccgcattcggcccactgggtgagtgaactgtaggagatgtggcctggatggtcaatggtggacggtttttgtgctagaggcattacttgtcgcctttgtgggtgttccatgggttacgggtgtcttggccggcgtggtgttgctggtatgcgaggagtgcgaggcagtgccaggcagcatgggctagatggggtagcccggattcatcatcgaggttgtttccttgctgccatgatagcaggtgcctgtagagggcgtcgacgctgtggctccacggatagccgccggtccagttgttgtcgccgtatttggtggcaccgtatcctgccacggagccgagggcgtgcaaggctgcggggtcgatgagggatagcctgcaaagtttcaattcttttttggcaccgctgttggggtcggtgtacatgcgggttggcttatccatgagatgtgtgctccttaggggtgggttactggttggggttgtgggcgagtgctacggcgagaataatgatggcgagggtttcagcgatcagtatgggtgttgtgatcatttagtgtttcggggattgttggtgagtgtggaggcgcctaggagggtggtgagggcgcatgcggcgatgatggcgagggctgccttgtgtggggtgccggttgcgtacatccatgtgatgatgccgccttggatccatgccagtgtggtgaagaaagtttcgtagctgtgtagctcaatgttgttgttgggtgtgttcatgcttgctcctgaagaatggtgttgatggttttgtaaatgttgtacaggtcggcttcgatggtttgtagctgtttgatttggtggtcgagatcaatgtctgggttgagggtgttgatgcgggaggcgatgtcggtggctgtgcgtagtgtgccgccggtgtggtgaatgatgtgtgccgtgtcggcgagtccggtggtgacggcgtagtgggagaggagaggcatagctggggggtgctccttggcgggttactgttgcgggttgatgttgaggtcggtgacgtgcgggtggtcttctgttccggtgacgaggcagtggacggtgacgggtagtttggatgctccgggctgtttcatggttgcgccgtagacgatggagaaggtgtctttgccaataattttgtggagttggaggtcgatgtcggggttgccgttccatttgacaccttgcgtggcggccttctgttcggctttgcggttgcaggtgtgtgcggcggtgatcatggtgagaccttgtgaggtttcttcaccccttgcctgggcttgcttgtgggttttggcctgctcggctcgcagtgactgttctgcggctgcctgccgtgccgctttctcggctttgcgctgttgggtggttttgggggtccattcggtgttggctgtggtggcctgtggggctggctgtgaggcgagtggcggattgtcgtctggggctggcatgaatgaggcggcggcgatgatggcggctgtgattccggcgatggtgtagcctgttttcttgttcatgactgttgtcccctttccggggtgttgttcgttgctgacatgatcaatcatggtgtgggcggttccccatgtcaaggctgcgctcaacgattgtgagcgatccttgtgtggctaggggtgatggcttctttcgcccaataggatgtgccaccgctggtccagtatccgagtttgttgcgctgcatgcccttcgcctccatctcgtccacggtgaggcacctgcggcgattggggcctgtcttgaccccgtggtcgcctacccggtgcatgtcgcctgaggtggtactcgtgaatgtctcgtgacagactgtgcagtgctctggcttgtatccgatgattgtgctatcgcacttgtggcatgtccattgcatgattgctcctattttccattataagacttcctgtagtgccattttagcgccttgcgagtcttgggggtacaactatataggtcaggtatttctaggcgattctaggctcattgtgtgcgactgggtgttatcgggcacacagggtgagcaggcttccgatgttgatgcggctcacattccagtagagttgtgtggcttcaccgccggtgagtggcttccactcgtcatggctgaacacggtgccatcggatgcgatgaatgtgttggggcgtagcttgtggagttcagtctctacgctctgccggtaggcttcggcgaggccctcaaaatccatgtggtcgcaggagaggttttcgaggcgtgtcaggtcgaaaggctcagggcagtccggggtgggggtgtaaagctgggtgaagtggttggcgatcttctgcatgacgggttccttttctcgtgtggtgggttgatagttttatcgtgtggcttcggcaatgatggcgtccacatcgatcatgtcgatgagatcgttgagttcctcggcctcgttctcggagaggtggcgccagtcgggtggcccgtatacggcaccgtcgagggtgacagtccacaggggccggatgagccgtacggcttcctcgactttggcacggtataggcggcagatgatagacgtgtgggtgttgcctatgtcacatcctgccaggtgtgcggggtggagtgggtggatttctgtctgcccgtagaggctggtgaaggatggtgtgatgagtgtgccatccatgagggtgtgctcctttcggtggtgtatgggttgttgtggtttctagagtgtgtagatggcgatcccatagtcaaggctgcgctcatttggattgagcgtttcatgggatgtggcaggggatgtggcgtatctcacttaagcctttatggcctctctcagcgcctgaaatcttctggcggtaggattatgcagggttgaccctgctaatcgattctaggggccttctagagcgtctcaggggtatgtctgggtgatagcgggtgtggcagatgatctagcgagtcaaggtgccgagctgagacataagatctatcatctaggtgtgtgagatgcatcacatcctcctggcgtggtgtacacccttaaggctactcggtcgatctggcgtggagggtgtagcccagaaatgccgtttaaagctgtgactcggtggattagagcgccttacagggtgggggctaggtatttatacccccaagcaattctgatcgattctagacgcctacaggagcccgatacacgatcagctatccagacgcagatcaccagtccctatcctggttagctcagcctacactatgtggacagtgtggaatactgtaggggaagaaggacacggtaaaagaaagagggaggcatcagccttcaagccttaaggtcttagcgcttagcaccgatggtcttagcagttagcaccgagcccctcaagggctcggcatcagcccgaacaggcacagccctgaaaggagtacacgccatcagggaaggcttgagagtacgaggagccctagcgacgagtactcgaaagcctgagggaacaccctcagcactgatgggcctagcgtgttcggaaaggacacaagagtgaagtgtgagagctgttcgggagtgaaacctgttctgactaggggtttcggtcttaaccaccctcaaaggttacaagactctaagaaaatttaaggaaaagtttaggtttaatttttggacctttactaccaaaaacacccgtttacacccctcaaacccgcctatagagccaaaaccaccagtttgactcatcccaggtggcatatgataggctggacaggtagccagctggacgcaaggccgaaatccgctgacgcggctttcacccttacatccatcagtctaccaaacactttaaagcttcaaggcttagcgctaagcccttaagaccttaacgcttagcaccgagcccctcaagggctcggcatcagtcttaaagctttaaacactttaagtaaacttaagagcttagcacttaaagttaattaataaccttaaaggcttagcacttaaggatataaacttaacatcagtgtttaagactttaaaacttaaaataactattaagacttaaagacttataagctttaaacacttaaagtaactataagactttaaagaccttaagtacttaaagttaaccatcagtcttaaactttaatattataacctataagtcttaaagcttataggtataataatataatataagttataaaagttttagaagagctaaggggttaacttctttacttctctactctctttggttctttctctcttctcttcttttcttcatcaggggagaagaggaacctttaccatcaacgctgatgggctttcaccgtgtgactcgtgtgcttctggtcgcacgctcccggttgcacactcaccacacttactctacctgtgtccctttcaggcttagcgtgttcggctgaaggcgtacggcgtgtcacgctaacacccttaacaccaggtaagacttaaagtgtatattataagtagaagactttaaaaccttaaggtgttcccgcttagcctgtgtcctttagcgctaggcgctaggcgctaagctgtgaaacgcgaacacccatccacccccttttttcttccgtgtccttcttcttttgacaccgctggggggcgatgtgatctttctcacactcatggggggtagtggagaaaacaaccaccccggcacaaacagaacaccccctcaaacgaacaaaacagcccctaggatcgatcagcagggcaagggcaaggtattcatacccccagacgattcccaggccgttacaggagcaatgagaggctcacaggggcaaggggagatcaggggacgtgatggcacataccaaccgcaccgcatccgcctcacaccggcgctggcggcaacgactcatcacccaagcccaacaacaaggccaaaccgaatgcccactctgcggagcaaccatcacctggggcacacacgatctaccaactagccccgaagccgaccacatcacacccgtcagcaggggaggactcaacaccctcgacaacgggcaaatcatctgcagaacatgcaacagaagcaaaggcaatcgcagcgaaccaaacatcaagttccaacaacaaaccacaaaaacattgattccatggtgaaaaacctgccaacccccaccggggacaccccctgcacacccgtgcaagacctcgtacggctt

SEQ ID NO: 2 - DNA, 29491 bp, Cutibacterium acnes (C. acnes) Bacteriophage Phi50M
agtgaaatacctcccttttttggttttgtctgtttgtcgactttttgtgttggtggtgagtgttgtgcagcctgagcttcctggtagtcgtgattggtgtggggagacgcgtcgttggtggcgtgtgtggggtgaggatagtcgcgcgccgtatgtgtctgatgaggagtggttgtttctcatggatgctgcggtgattcatgattgtgtgtggcgtgagggtcgcgcggatttggtggcttcgcttcgtgctcatgtgaaggcttttatgggcatgttggatcgttattcggttgatgtggcgtctggtggccggggtgggggttctgcggtggcgatgattgaccggtataggaagcgcaagggggcctgattaggtgtctggtgttgtgggttctcaggttcctcgtcatcgtgtggctgcggcgtattcggtgtctgctggcggtgatgctggggagttgggtcgtgcgtatgggttgacgcctgatccgtggcagcagcaggtgttggatgattggcttgctgttggtagcaatggcaggcttgcttcgggtgtgtgtggggtgtttgtgcctcgccagaatggcaagaacgcgatccttgaggttgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgggcgacgaatggtcaggaggctattgtgttgcatcacccggattgtgccacgtttgagaagaagtgtggctgtccgggttggggttcggttgagtttgtggcccgttctcggggttcggctcgcgggtttacggttgatgatttggtgtgtgatgaggctcaggagttgtcggatgagcagttggaggcgttgcttcctacagtaagcgctgccccgtctggtgatccgcagcagattttccttggcacgccgcctgggccgttggcggatggttcggttgtgttgcgtttgcgtgggcaggctttgtcgggtggtaaaaggtttgcgtggacggagttttcgattcctgacgagtctgatccggatgatgtgtcacgccagtggcggaagcttgcgggggatacgaatccggcgttgggtcgccgcctgaatttcgggaccgtaagcgatgagcatgagtcgatgtctgctgccgggtttgctcgggagcggcttggatggtgggatcgtggccagtctgctgcgtctgtgattccggcggataagtgggctcagtctgctgtggatgaggcgagtctggttggcgggaaagtgtttggtgtctcgttttctcgttcgggggatcgtgtcgctttggcgggtgccggccggactgatgctggggttcatgttgaggttattgatgggctgtcgggaacgattgttgatggtgtgggccggttggcggactggttggcggttcgttggggtgatactggccggatcatggttgccgggtctggtgcggtgttgttgcagaaggcgttgacggatcgtggtgttccgggccgtggcgtggtggttgccgatactggcatctatgtggaggcgtgtcaagccttcctggaaggtgtaaggtctgggaatgtttctcatcctcgtgctgattcgaggcgtgacatgttggatattgctgtgaggtcggcggttcagaaaaagaaaggctctgcgtggggttggggttcctcgtttaaggatggttctgaggttcctttggaggctgtgtctttggcgtttttgggggctaaacgtgttcgtcgtggccgtcgggatcgtagtggtaggaagcgggtgtctgtggtatgaacgtggacgagttggctctgattgagggcatgtacgatcgtatccaaaggttgtcttcgtggcattgtcgtattgagggctactatgagggttctagccgggtgcgtgatttgggggtggctattcctccggagttgcagcgtgtgcagactgtggtgtcgtggcctggtatagctgtggatgctttggaggagcgtctggattggctgggctggactaatggtgacggctacggcctggatggtgtgtatgctgcgaatcggcttgctacggcgtcgtgtgatgtgcatttggatgcgctgatttttggtttgtcgtttgtggctgttattccccaggatgatgggtcggtgttggttcgtccgcagtcaccaaagaattgtactggccggttttcggctgacggttctcgtttggatgcgggtttggtggtgcagcagacgtgtgatcctgaggttgttgaggcggagttgttgctgcctgatgtgattgttcaggtggagcggcgtgggtctcgtgagtgggttgagacgggccgtataccgaatgtgttgggtgctgttccgttggtgcctgttgtgaatcgtcgccgtacgtcgaggattgatggccgttcggagatcactcggtcgattcgtgcttacacggatgaggctgttcgcacactgttggggcagtctgtgaatcgtgacttctatgcgtatcctcagcgttgggtgactggcgtgagcgcggatgagttttcgcaacctggctgggtgttgtcgatggcttctgtgtgggctgtggataaggatgatgatggtgacaccccgaatgtggggtcgtttcctgtgaattctcctacaccgtattcggatcagatgcgtttgttggcgcagttgactgcgggtgaggcggctgttccggaacgctatttcgggtttatcacgtctaacccgccttctggggaggctttggctgcggaggagtctcggcttgtgaagcgtgctgaacgcaggcagacgtcgtttggtcagggttggttgtcggttggtttcctggctgcccgggcgttggattcgagtgttgatgaggccgcgttttttggtgatgttggtttgcgttggcgtgatgcttcgacgcctacccgggcggctacggctgatgctgtgacgaagcttgttggtgccggtattttgcctgctgattctcgtacggtgttggagatgttgggtttggatgatgtgcaggttgaggctgtgatgcggcatcgtgccgagtcttcggatccgttggcggcgctggctggggctatttctcgtcaaactaacgaggtttgataggcgatggcttcgggggctatgtcgaggcttgctgcgactgagtatcagcgtgaggctgtcaggtttgctgggaagtatgcgggctattattctgagcttggtcgtttgtggcgtgccggcaggatgagtgacacgcagtatgttcgtttgtgtgtggagttggagcgtgccggccatgatggttcggcatcgttggctgccaggtttgtgtcggattttcgccggttgaatggtgtggatccgggtttgattgtgtatgacgagtttgatgctgcggcggctttggctaggtcgttttcgactatgaagattcttgagagtgacccggatagggcgaatgacacgattgatgcgatggctgcgggttttgatcgggctgtcatgaatgctggccgtgacacggttgagtggtctgcgggtgcgcagggtaggtcgtggcgcagggtgactgatggtgatccgtgcgcgttttgtgccatgttggctacgaggtcggattatacgactaaggaaagggcgcttactactggtcatacgcggcgtcataagcgtgccggtaggcgtccgtttggttcgaagtatcatgatcattgtggttgtacggtggttgaggttgttggcccttgggaaccaaatagggctgatgccgagtctcagaggacgtatgagaaggcccgtgagtgggttgatgatcatgggttgcagcagtcgcctggcaatattttgaaggctatgcgtactgttggcgacatgagatgatggtttccggttgtgtgccgccggttatcggtgcacagggttgtctcccgcacgggggtcaacaatgttgtgttgttttccgcaaggagtatagggttaggctatggccgatcaaaaagttgaagaacagaatgttgacaatgatgctgttgagcccggaaagggtggagacgttgttgatgttgtgaaggatgggcaggcttccggcgatgatcatgccggtgatgtttccgtgaaggaggagtcttcttctggcacggattggaaggctgaggcccgtaagtgggagtctcgtgctaaaagtaatttcgccgagttggagaagcttcgtacatcgagtgacgattctggatctactattgatgagcttcgccgcaagaatgaggaactcgaagacaggatcaacgggtttgttcttgagggtgtgaagcgcgaggtggcttcagagtatggtttgtccagtgatgcggtcgctttcttgcacggtggcgatcgtgaagcgctggtggagtctgctaaggctttgaagggtttgatcgaccagagtggtggtggcgcgggtgtgcgccgtcttgcggggagtgcccccgttgatgatgttaaacgacgtgagggtgtcgcgtttgtggatgctcttgtcaataattctaggagatgatttctgatggctgacgattttctttctgcagggaagcttgagcttcctggttctatgattggtgcggttcgtgaccgtgctatcgattctggtgttttggcgaaactgtcgccggagcagccgactatttttggcccggtgaagggtgccgtgtttagtggtgttcctcgcgctaagattgttggtgagggcgaggttaagccttccgcttcggttgatgtttcggcgtttactgcgcagcctatcaaggttgtgactcagcagcgtgtctcggacgagtttatgtgggctgatgctgattaccgtctgggtgttttgcaggatctgatttctcccgctcttggtgcttctattggtcgcgccgtggatctgattgctttccatggtgttgatccggctacgggtaagcctgctgcggctgtcaaggtgtcgctggataagacgaagaagacggttgatgccaccgattccgctacgactgatcttgttaaggctgtcggcttgattgcgggtgccggtttgcaggttcctaacggggtcgccctggatccggcgttctcgtttgctctgtctaccgaggtgtatccgaaggggtctccgcttgccggtcagcctatgtatcctgccgccgggtttgccggtttggataattggcgtgggctgaatgttggtgcttcttcgactgtttcgggtgccccggagatgtcgcctgcctctggtgttaaggctattgttggtgatttctcgcgtgttcattggggtttccagcgcaatttcccgatcgagctgatcgagtatggtgatccggatcagactgggcgtgacctgaagggccataatgaggttatggttcgcgccgaggctgtgctgtatgtggctatcgagtcgcttgattcgtttgctgttgtgaaggagaaggctgcaccgactcctcctccggccccgaagcctgagcctaatcctccgtctggtaactgatacaagataagcgaatgtgtactatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgcagggctttgttgcgctggaatgataccggggtttcgggtcaggtgcagtatgagtctgcgggtcctttcgctcagactacacggtctaatactcccacgaatttgttgtggccttctgagattgccgcgttgaagaagctgtgtgagggtgatggtggggctggtaaagcgttcactatcacacccactattaatggtcgatatgtgcattctgaggtgtgttccacggtgtggggtgagggttgctcgtgcggatctgatattaacggctacgctggccctttgtgggagatatgatatgaccagttttccttatggtgaaacggttgtgatgcttcagccgactgttcgtgtcgatgatcttggcgacaaggtggaagactggtctaagcctgtcgagactgtgtaccataacgtggccgtttatgcttccgtttcgcaggaggatgaggcggctgggcgtgactctgactatgagcattggtcgatgctttttaagcagtctgttgtgggtgccggttatcgttgccggtggcgtattcggggtgttgtgtgggaggctgacgggtctcctatcgtgtggcatcacccgatgtctggctgggatgcgggcacgcagatcaatgtgaagcgtaagaagggctgatagattgtggctcaggatgtgaatgtgaagctgaacttgccgggtattcgtgaggtgttgaagtcttttggggtgcaggctatgttggctgagcgtggcgagcgtgtcaagcgtgcagcctcggcgaatgtgggcggtaacgctttcgataaggcccagtatcgtgcagggttatcatcggaggtgcaggttcaccgtgttgaggctgtggcccgtattggcaccacatataagggtgggaagcgtattgaggcgaagcatggcacgctggctcgttcgattggggctgcgtcgtgattgtttacggtgaccccaggaagtgggctaaacgcgtgctcaaggatgatggctggctgtctgggataccgtgtacagggacggtgcccgatgattttacgggtgatctgatttggttggctcttgatggtggcccgcagttgcatgttcgtgagcgtgtttttttgcgggtgaatgtgttttctgatatgccggatcgtgctatgtctttggctcgtcgtgttgaggctgtgctggctgatggtgtggacggtgaccctgtggtgtactgtaggcgttctactggacctgatttgctggttgatggtgcacgttttgatgtgtattcgctttttgagcttatatgtaggcctgcggagtctgaataagcttattgtttttgttttaatgtaattgtttgatatttaatgggggttatgatggctgcaacacgtaaagcgtctaatgttcgctctgctgttacgggtgacgtctatattggtaaagctcatgccggtgacactattgatggtgtgaagacggttcctgacggtcttaccgctttagggtacctgtcggatgacgggtttaagattaagcctgagcgtaaaacggatgatttgaaggcttggcagaatgcggatgttgttcgcactgtggctacagagtcgtctatcgagatctctttccagctgatcgagtctaagaaggaggttatcgaactgttttggcagtcgaaggttactgccggagccgattctggttcgttcgatatttctcctggtgccaccactggcgtgcacgccctgttgatggatattattgatggcgatcaggttattcgctactatttccctgaggtcgagttgatcgatcgtgacgagatcaagggcaagaatggcgaggtgtatgggtatggtgtgacgttgaaggcgtatcctgcccagattaataagaagggtgatgcggtgtctggtcgggggtggatgacggctttaaaagctgatactcccccggttcctccttctccgaagccggagcctcctaagcctgagccggatccgaatccgccgtctaataactgatacacatagtttgagggattgttgatagatgagtgacacaggttacacgttgaagattggtgaccgtagctgggtgttggcggatgcggaggaaacagcgcaggctgttcctgcccgcgtttttcgccgtgccgccaggattgcccagtctggggagtctgctgatttcgcccaggttgaggtgatgttttccatgttggaggctgccgccccagaggatgctgtggaggccttggaggggcttcctatggttcgtgtggccgagattttccgtgagtggatggaatataagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccacctgaatggttggcagcgcccgtttgagtggtgcgagtgggctgtgctggacatgctggatcattacaggtctgctaatagtgaggggcagccggagcctgtggcgaggcctacggatgagcgtagggcccggtttacgtctgggcaggtggacgatattttggcgcgtgttcgtgccggtggcggggtgtctcgcgagattaatattatggggtgaatagtgtatgtctggtgagattgcttccgcatatgtgtcgttgtatacgaagatgcctggccttaaaagtgatgttggtaaacagttgtcgggtgtgatgcctgctgagggtcagcgttcgggtagcttgtttgctggcgggatgaagttggcgcttggtggtgcggcgatgatgggtgccatcaatgttgctaagaagggcctcaagtctatctatgatgtgactattggtggcggtatagctagggcgatggctattgatgaggctcaggctaaactgactggtttgggtcatacgtcgtctgacacgtcttcgattatgaattcggctattgaggctgtgactggtacgtcgtatgcgttgggtgatgcggcgtctacggctgcggcgttgtctgcttcgggtgtgcagtctggcgggcagatgacggatgtgttgaagactatcgccgatgtgtcttatatttcgggtaagtcgtttcaggatacgggcgctatttttacgtctgtgatggctcgcggtaagttgcagggcgatgacatgttgcagcttacgatggcgggtgttcctgtgctgtctctgttggctaggcagacgggtaaaacgtctgctgaggtgtcgcagatggtgtcgaaggggcagattgattttaacacgtttgcggctgcgatgaagcttggcatgggtggtgctgcgcaggcgtctggtaagacgtttgagggcgctatgaagaatgttaagagcgccctgggctatcttggtgctacggctatggccccgtttttgaacggcctgcggcagatttttgttgcgttgaatccggttattaagtctatcacggagtctgtgaagcctttgtttgcgtcggtggatcaggggattcagcggatgatgccgtctattttggcgtggattaaccggatgccggctatgatcacaagaatgaatgcacagatgcgcgccaaggtggagcagttgaagggcatttttgcgagaatgcatttgcctgtccctaaagtgaatttggctgccatgtttgctggcggcaccgcagtgttcggtcttgtggctgccggtgtggggaagcttgttgcggggtttgccccgctggcggtgtcgttgaagaatctactgccgtcgtttggtgctttgaagggtgccgctggggggcttggcggcgtgtttcgcgccctgggtggccctgtcggtattgtgatcggcttgtttgcggcaatgtttgctacgaacgcccagttccgtgccgcggtgatgcagcttgtggctgtggttggccaggctttggggcagattatggccgctgtgcagcctgtgtttggtctaattgctggtttggtggcacggttggctcccgtttttggccagattatcggtatggttgccgggttggctgcgcagcttatgcctgtgattagtatgcttgtcgcccggctggttcctgttatcacgcagattattggtgcggtgacgcaggttgctgcaatgttgttgccggcgttgatgccggtgttgcaggctgttgttgctgtgatacggcaggttgttggcgtggtcatgcagttggtgcctgttttgatgcctgtgattcagcagattttgggtgctgtcatgtctgtgctgccacctattattggtcttatccggtcgttgatgcctgtgattgcggcggttatgcgtgtggtggttcaggttgttgcggttgtgatacaggtggtggcccgtattcttgctgttgtggctccgatggtggcggccgtggtcgggtttgttgcccgtattgttggtgctgtcgtgtcggctgtggcccgtgttattgccgctgttgcccgtgtcatctcgtgggttgttgcccattttgtgtctggtttggcgcgtatgggttcggtggttcaggctggctggaatcatattagggcgtttacgtctgcgtttattaacggtttcaagtcggtgatttctggcggcgtgaacgcggttgtggggttttttgcccggcttggttcttcggttgcctcccatgtgaggtctggttttaacgcggctcgtggcgctgtttcttctgcgatgaatgctatccggagtgttgtgtcttcggtggcgtctgctgttggcgggtttttcagttcgatggcgtctagggttcgtagtggtgctgtgcgcgggtttaatggtgcccgtagtgcggcttcttctgctatgcacgctatgggctcggctgtgtctagtggtgtgcatagtgtgatagggtttttccggaatctgcccagcaatattaggggcgctttgggtagtatggggtccctgttgatgtctgctggccgtgacgtggtggccggtttgggtaatggtattaagaatgctatgagtggcctgttggatacggtgcgtaatatgggttctcaggttgctaatgcggcgaagtcggtgttgggtattcattccccgtctcgggtgtttcgtgacgaggttggccggcaggttgttgctggtttggctgagggtattactggtaatgctggtttggcgttggatgcgatgtcgggtgttgcttcgcagcttccggatgctgtggatgcccggtttggtgtgcgatcgtctgtgggctcgtttaccccgtacgaccggtatcggcgtgctcagggtgagagtgttgtggtgaatgtgaatggcccgacgtatggtgatccggccgagtttgcgaagcggattgagcggcagcagcgtgacgctttgaacgcgttggcttacatgtgagtaaaggggtatgcatgtttattcctgacccgtctgatcgttctggtttgactgtgacatggtcgatggatccgcggtttggcgacgagcgtgtgcttcatttgacggattatacggggtcgtctcctgtcatgttgttgaatgattcgttgcgcggtttgggtgttcctgaggtggagcatttttctcaaacgcatgttggggtgcacggttcggagtggcgcgggtttaatgtgaagcctcgcgaggtgacgctgccggtgttggtgtcgggtgttgacccggatccggtgggcgggtttcgtgacggttttttgaaagcctatgacgagttgtggtctgcgtttcctcctggcgaggagggggagttgtctgtgcagactcctgccggtcgtgagcgtgtgttgaagtgccggtttgattcggtggatgacacgtttacggttgatccggtgaatcgcggctatgcccgctatgtgattcatttgacggcctatgacccgttttggtatggggatgagcagaagtttcgttttagtaacgctaagttgcaggattggctgggtggcggccctgtcggcaaggatggcacggcgtttcctgtggtgttaacaccgggtgtgggctcgggttgggataatctgtctaataagggtgatgtgcctgcgtggcctgtgattcgtgttgagggcccgttggagtcgtggtctgtgcagattgatggtttgcgtgtgtcttcggattatcctgtcgaggagtatgattggatcactattgatacggatcctcgccagcagtcggcgttgttgaacgggtttgaggatgtgatggatcgtttgacagagtgggagtttgcgcctatcccgcctggcggttcgaagagtgtgaatattgagatggttggtttgggtgccattgttgtgtcggtgcagtacaggtttttgagggcttggtgaatggttgatggctggtcttgttccgcatgtaacgttgtttacgccggattatcgccgtgtggcgcctatcaatttttttgagtcgttgaaactgtcgttgaagtggaatggtttgtccactttggagttggtggtgtcgggtgatcattctaggcttgacgggttgactaggccgggtgcacggctggttgttgattatggtggtggccagattttttctgggcctgtgcgtcgggttcatggtgtgggtccttggcagtcttcgcgggtgactatcacgtgtgaggatgatatccgcatgttgtggcgtatgctgttgtggcctgtgccttatcgttccagcatcattgggatggagtggcgtgccaatagggattatgcccactattcgggtgcggcggagtcggtcgctaagaaggcgttgagggataattcttggcgttttccgcctgatatatttatggtgaatgataagagtcgtggccgctatattaaggattttcaggcgcggtttcacgtgtttgccgataagttgttgccggtgttgtcgtgggctcggatgactgtcacggtgaaacagtttgagaatgtgaagcaggatcagcgtggtttactgtttgattgtgtgcctgctgtgacgcgtgagcatgtgttgactgccgagtctgggtctattgtgtcgtgggagtatgtgcgtgacgccccgaaggctacggcggtggttgtgggtggccgcggcgaaggcaaagatcgtctgttttgtgaggattttgacgcgttggccgaggatgagtggtttgatcgtgtcgaggtgtttaaggatgcccgtaacacggattctgagcatgtgcatctcattgatgaggcggagcgggtgttgtccgagtcgggggccacgtcggggtttaagatcgagttggctgagtcggatgtgttgcggtttgggccaggcaatctgatgccgggtgatttgatctatgtggatgtgggctcggggcctattgcggagattgttcggcagattgatgtggagtgtgtatcgcctggtgacgggtggacgaaggtgactcctattgcgggggattatgaggataatccgtcggccctgttggctcgccgtgtggctgatttggctgcgggtgtgcgggatttgcaaaaattttagaaaagaatcgggggtttgttgtgggtattgtgtgcaagggttttgatggtgtgttgaccgagtatgattgggctcaaatgtctggtctgatgggtaatatgccgtctgtgaagggcccggacgattttcgtgtcggcactactgttcagggtgccacagtgttgtgtgaggtgttgccggggcaggcttgggctcacggggtgatgtgcacgtcgaatagtgttgagacggtgacagggcagctgcctggtcctggcgagacccgatacgactatgttgtcctgtctcgggattgggagcagaatacggccaagttggagattgttcctggggggcgtgcggagcgtgccagggatgtgttgagggctgagcctggcgtgtaccatcagcagttgttggcgactttggtgttgtcgtctaacgggttgcagcagcagttggataggcgtgctgtggcggcccgtgtggcgtttggggagtctgctgcgtgtgaccctacccctgtggagggtgaccgggtgatggtgccttcgggggctgtgtgggctaaccatgctaacgagtggatgctgttgtccccgcgtatcgagacgggttcgaagcagatccagtttggcggggctgctgtgtatgcttacacgatcccgtttggccgcccgtttggtagtgcgcctgtggtggtggcgtctatggcgacggcggctggtggtacgcagcagattgatgtgaaagcctacaatgttactagcaaggattttcagttggcgtttatcacgaatgatggttctaagccttctggtgtgcctgcggttgctaactggattgctgtcggcgtgtaatgtacggcttgcgggtatgtgacgtgttgtggttgttgtagtggtagtgggctgtagtgtcatggcttacacccacactcgtagcctctctttgtaccgctatcgctactgttcttggttcgattcaggcggctacgtacaggtcgaagaagaggctgaggcagttgtctgcgcaggttgatgcgatggaagaatacacgtggaatattcgccatactgttcaccgctataatgcgaatctgccggagaatgttgagccggtaaagatgcctgatttgcccgagtttttgaaggatactgttgatggtggtggggggtgaattgtgagggagttggaggaagagaagcggcagcgccgctcgtttgagaaggcttccatgatactgttgttcctgtcgcttgtactactggcggtggtcgccgggggtgctttacgtttcggtgctgtggcttcccagcgggattcggagcaggctaaagcccagtctaatggtacagccgctaaagggttggctagccgtgtgcggcaggtgtgtgcctctggcgggcaggagtcggtgcggcttcaccagtctggcttgtgtgtggatgctcagcgtgttgagcgtagcgtgcagggtgtgcctggtcctgccggtgtgcgtggcccgcaagggcctgcaggggttgacggccgggatggtgttgatggttcggctgggctggttggccctgttggcccgcagggttcccctggtttgaatggtgtgaagggtcctgacgggctgcccggtgctaacggttcggatggccgtgatggtgttgccggtgtgaacggggctgatggcaaggatggtgtgccaggtagggatggcgctgatggggctgatggtggccgcggccctgttgggcctcaggggccgcagggttctgccggtgccgatggcaaggacggtaaggatggcgctgatgggcgctcggtgatatcggtgtactgttccgggggccgcctggttgtgaaatatagtgacggtacggcctctaccatagcgggttcggtggcctgtgagggtgtgaaaccatcaccggtggttaccgtatcatcccacaagtaaaagaggaagtgtgttactagtgttgatagtagtgtttgggggtggcgtgttgtgagatacattccagcggcgcatcactcggccggatcaaataagccggtgaaccgtgttgtgattcacgcgacatgcccggatgtggggtttccgtccgcttcccgtaaggggcgggcggtgtctacagcaaactattttgcttccccatcgtcgggtggttctgcccattatgtgtgtgatgttggggagacggtgcagtgcttgtcggagtctacgattggttggcatgccccgccgaatccgcattctttgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtgatggtggtttgttgtgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcataagtgaatatagatgtgtgccccagcggtgctgccacgatcgtgtggtggttgccgctggggcactatttttgtgtctatagtattctatgattcgttgctgtcgatggtttcttcgatcagctggtccaggtggatgcaggcggagatagtatcgttggcctggtgcagaacgttctggccgataacatttttataattgtcgcggtggcagatgatagactgcatgatatcgtcggcttccgcttgcaatagtttggcctggtatgcgattccggcgagccaatctatggcttcctggcttgcccgtgtgtcgtctggaatgccacgggtgttgctgttgtttgggtatcctgcactgtcgcagtcccacaagatttcgctgcactcgtctagcgtgtcctgatcgatagcgagatcgtcgaggctgacttctttgacggtaaggttcacgttgtcgagtgagatgggtacacggtactggttttcgacaccgtcaacaatgttttccaattgctgcatgttggtgggctgttgttggatgattcggtgtactactgttttgagggcggtgtaggggatattgtgtgtgttgtccatcgtgttatgccattccttcgttatcgtctggcatgtagtatgtgctgtttgcgtactcggttaacgtcatcagtgtttggtctgcccactgtttcacggtttgccgggtgactccgagtcgttgggctgccaccgaataggtttggtcatacccgtagacttcccggaacgctgccaacctagctagccgtttcctttgtttggatggctggcaggtgagggtgtagtcgtcgatggctagttgtagatcgatcatggtgacaatgttgttgccgtggtgttgtggcgcggtgggtggtggtggcattcctggttcaacactcggtttccatggtccgccgttccagatccattgcgcggcttggataatatcggctgtggtgtaggtccggttcatgtgtcatcccctgaataggttgtcgaggttgtctgggttgctggtgtcgaatcgtccgacgcagtggcagtagtcgtacatgagtttgatgatgtgttggtggtctcccaaataggtgtttccgctgatgctgtaggtggctgtgccgtctttactaatagtgtatttggcggtgatggtttcgggtgtttcggtgttggtgatgatggcggtggtggtggcgcctactgtttgtagcctggtggtttgggtgccgtcgtcgaggatggtggtgaccattatgattctccttagttgctggtttggttgtcggctagatgaataatatcggataaaggtttcggctggtctaaatgttgtgtggttttgttggctagccgtttggctaccctgtagcacattttggtgtagtgtttgttgtctaggttgtggtattgttcccgcaccgcaatatatagtagggagtcttggtagaggtcgtctgcgctgattgcggggtagtgtgtaaccactttggtgcatgcccggttgagtgtgcgtagatgatggtctgtggcccatccccacgatgcggtggtggctaggtctgcttttgttggtcgcctgctcatggcactattacctggctatctggtagttgtttggtgttttgttgttgatagtgtagcacacgagtccggggtttccggtggtgcccgtcttgtgccggaaccatgtggattcgccttccatggatgggcattggatgaaggtgcgtgtgccttgctcagagatttctaggtggtgccggtgcccggccatcaggatgtgggatgtggtgccgttgtggaattcttggccgcgccaccattcgtagtgtttgccggtgcgccattggtgcccgtgggcgtgcaggatttgtgtgcctgccacatcgacggtggtggtcatttcgtctcggctggggaagtggaagtgaaggttggggtattggttgttgagctggtaggcttctgcgatggcgcggcagcagtccacgtcgaaggagtcgtcgtaggtggtgactccttttccgaagcgtacggcttctccgtggttgccggggatggatgtgatggtgacgttggcgcagtggtcgaattggtggatgagttgcatcatggccatgcgggtgagccggatttgttccgtcaggggtgtttgtgtgcgccaggcgttgttgcctccttgtgacacgtatccttcgatcatgtcgccgaggaaggcgatgtggactcgttgcggtttgcctgcttgctgccagtagtgttttgcgacgatgagggtgtgtaggtagttgtcggcgaagtgtgctgtttctccgccggggatgcctttgccgatttggaagtctcctgccccgatgatgaaggctgcagtgctgtagtcggtgtgggtgtcttgttcgggttttggtggctgccattcggctagtttatcgacgagttcgtctatagggtaggggttggttgcgggttggtggtcgatgattttttgtatggatcggcctgtttctccgttggggagtgtccattcggagatgcgtgtgcggcgcacggtgccgttggctagattgtcgtcgatggtgtcgatggcgttgtcgtggttggctagctgtgtgaggagccggtctatgttgtctatcactgggtatcatcctcttcctcgtgtgtggtggtggcttgtttgcggcgatggtctttgataacggtggcggagatggggtatccggcttcagtgagcatttgggctagctgtgtggcggggatggacctgtcggcgaggacgtctgcggctttgttgccgtagcgttggatgagggtttcagttttggttgccatgatgtcctatcggttgtgtggtgggttgccatcctgtgcggcagtcgccgtcgtgtcctggtttgcgtgtgcaccatgagacttcgccggcattgtggatgatggcacggccgcatatgacgtcatgtaggtgttcgggaaacttatcgttgttgttgtccccgtacatgtcgatcaagtgttgggttttagtaaccatcatgtctcctatgtgtgaaagagtgtgcaaatactatgctggtgtcatggatgtttatgcgggtatggttttcatcaccttgctgaacgttacttggttactgtacatcatctgggtgatttcctgatccgttttgtcggggtgctgttttcgcaggtttgcccattggcaggcgttgtcggtttcctgctgtaaacgtgtcaggtgttgttcgttgatgatgtgtttccacattgtccacgagacgtcgagtcgtttgagcatgtcgatggctggcacgttgaaggagttgaggaagagtatttcctccgtgtagtagtctttttcgtattggtcccatccgcttcggtgcctgttgggctggtttttagggtaggcttcccggcatactttgtgcaaacgcttggccatgtctttgggtagcctaatgtcggggttggcgcggatcatggatcgcatcccatcataggtggtgccccaggtgtgcatgatgtggagtgggtcttcaccatcagcccatttttctgcacagatggcgaggcggatacgcctccgggcggctttgctagtgtcgcggcggccggggatggggcatgtgtcgagggggtccatgatgctttttatgcctttctttgtttggtttgcttgtgtggttttattgtagcactgtgtctagtgcttgtgtcaaccctgtttttccggcctgcaggtaggtgtctgtgacatcgcccagggtgaggggcacgcgggtggcttgggggagtgccgtctggagggtttgggccatctggtctcctgctttgtcggggtctgaccagatgtagatgtggtcgtagccttcgaagaatttggtccaaaagttttgccacgaggttgcgccgggtagggctacggccggccatccgcattgttcgaggatcatggagtcgaattcgccttcgcaaatgtgcatttcggctgccgggttggccatggcggccatgttgtagatggagcctgtgtcccctgccggggttagatatttggggtggttgtgggttttgcaatcatgctggagtgagcagcggaaacgcatttttcgtatttcggctgggccgtcccagacggggtacatgtaggggatggtgatgcactggttgtagtcttcgtggcctgggatggggtcattgtcgatgtatccaaggtggtggtagcgggctgtttcttcgctgatgcctcttgccgagagcaggtcgagtatgttttcgaggtgggtttcgtagcgggctgaggctttctggattcggcggcgttccgcaatgttgtagggttgtagtgagtcgtacattcgggttttctttctctaattgttgtttcagtttggcgaggcctcctccgatgccgcatgtgtggcagtaccagacgcccttgtcgaggttgatgctcatggagggctggtggtcgtcgtggaacgggcagaggatgtgttgctcgttcctggatggattgtaccgtatctggtaggtgttgaggaggcggcaggtgtcagaggtgtgggaggagctcgttgagggttgataccacataggcttcgctccatggcttgttgcgctgtttcatcactacgagtccgatggtggaattgttttctcggtttcggtgggtttcgtagttgcgtgcctcccggctggcttgtttcacgaattcggcgaggtgtgcctgtcctgctttggcttcgatcacataggttttgtggccggttgtgaggatgaggtcgccttcatcctctttaccgttgaggtggaggcgttctatatcatagccggtgtcgcgtagctggtggaggagtcgtgtttcccattcggctccggcccggcggttgcgtgcctgttgtgttgacatgatagtcctttgtggtgttcggtcatgttccatggctgtttttcggcgagtggcccgaagaatgtgtattcggggtaggctcgcagtcgttcgtatcgggtgccgtctgggctggatttgcctgtgcgctgtttgagtacggcgatgcgtgcctctgccggtatcgtgagcccgttgccgttatcttcgccaccatacagggagactcccaatatgagttgtggtttttcggagaggccgtttttgatttcccgcctagccggggggtgttcgatgtcggtgccggttttgtcggttgcgtggtgtgtgacaataatggtggagccagtatccctacccaatgctgtgatccattgcatggcttcttgctgtgcctggtagtcggattcgcagtcttggatgtccatcaggttgtcgatcacaatgagtggtgggaaggtgttccacatttccatgtaggcttgcagttccatggtgatgtctgtccatgtgatgggtgactggaatgagaatgtgatgtgttggccgtggtggatgctgtctcgatagtattctggcccgtagtcgtcgatgttttgttgtatctgggcggtggtgtgttgagtgttgagtgagatgattcgtgtggaggcctcccagggtgtcatgtcccctgatatgtagagtgcgggctggttgagcatcgctgtgatgaacatggctagcccggatttttggctgccggaccgccccgcgatcatgacgagatcccctttgtgaatgtgcatgtccaggttgcggtagaggggttctagttgtggtatgcggggcagttcggctgcggtttgggaggctctctcgaaggatctttggagagagagcatcggagccttaatctatctgtctatcggttggatgatgttttggtggtcagatggagtcgatgtcgatgtcagcatcagcaggggctgtggtgtcgtctagctggccgttatcgcgtttgtctacgtattcggcaaccttatcgtagatggcgtcgtctaatggtttgagcacgaccgcgttgaacccgtttttggtgcgcacggtggcgagtttgaatgcctgttcttcgccaaggtaggcttctaggtcgcggatcatggagtgtgggcggtcgttgttgccgcgggctttctcgataatggcgttggggatggtttctggggtgccgttgttgagatcgtctagggtgtggaagattgtgacatcagcgtagatgcgatcggcgacctgtccaccgtagccttcggtgttgtgctggacgtcgcggattttgaaggcgatggcggtggcgtcctggtttcgggaggggttgaagaaggtgctgttgctgttgttgcggtagtttgcgagtcccattgttgtttcctttactgttttgttggtttgtttgttggttttatcgggtgaggctgtttcgtttgctgcggaaagcctcggaaacgtcactgttactggtgatgatctttttgtactgtttgaggaggtcggctagctgtgctttgcttgttgcattgttgattttgtcgatgatggtgttgtttccttctgaggcgatgttgtctacgtagtctttggcggcctggttgtatcggtcttggaggattatggatgctgtggcgatcagtgttgccaggtcccagttccttgccgcggagctgtttttgagtccgccgaggaggtcgatgatagtcttctttacctggtcggcggtgtctccgcggatgacggtccatggggcggcgtagtcgcctccgtatttgagggtgacggtgaatcggtcgtcgtctgtgttgtcggtcactggtgctccttgtcttcttttgttggggctgtgatggtggtttctatggggtacctgtaggcgtctttcccgttgacagcccagcaggcgtccttgacggggcagcctttacagagtgctgtgacgtggggtacgaagatgccttggctgattcctttcattgcttgactgtacatggatgatacatgccggtaggtgttgttgtcaaggtcatagagttcggttgctgtgccttgtgtcggagactggtcgtcgttgcggctggtggctggcgtccaaaacatgcctttcgtcacatcgttgccgtgttggttgagcatgtaccggtatgtgtgcagctgcatactgtcggcgggtaggcgtccggttttgaggtccaaaatgaaggtttcaccggtgtcagtgtcggtgaaaacgcggtcgatgtagccgacaatagtggtgccatcctggagggtggtttctaccgggtattcgatgcccggctggccgtcaataacagcggtgatgtattctgggtggttgcgcctccatgttttccaccggttgacgaaggtggggccgtatatcatccaccaattgtagtcttttttgtgtggcccgcccgattcgcacatgtttttgcatattcggccggagggtttgatgtttgtgccttctgatttggcgagggcgacttgggtgtcgaaaatgtttttgaaggatgagagtttgtctggcagtgcagggtattcggcgggattgtacaggtgtaggtcgtattgttcggtgatgtggtgtatggcgcttccggcgatggtggcgtaccaggtgtggtgttgggtgtggtagccgtgggataggcgccatttttctccgcattcggcccactgggtgagtgaactgtaggagatgtggcctggatggtcaatggtggacggtttttgtgctagaggcattacttgtcgcctttgtgggtgttccatgggttacgggtgtcttggccggcgtggtgttgctggtatgcgaggagtgcgaggcagtgccaggcagcatgggctagatggggtagcccggattcatcatcgaggttgtttccttgctgccatgatagcaggtgcctgtagagggcgtcgacgctgtggctccacggatagccgccggtccagttgttgtcgccgtatttggtggcaccgtatcctgccacggagccgagggcgtgcaaggctgcggggtcgatgagggatagcctgcaaagtttcaattcttttttggcaccgctgttggggtcggtgtacatgcgggttggcttatccatgagatgtgtgctccttaggggtgggttactggttggggttgtgggcgagtgctacggcgagaataatgatggcgagggtttcagcgatcagtatgggtgttgtgatcatttagtgtttcggggattgttggtgagtgtggaggcgcctaggagggtggtgagggcgcatgcggcgatgatggcgagggctgccttgtgtggggtgccggttgcgtacatccatgtgatgatgccgccttggatccatgccagtgtggtgaagaaagtttcgtagctgtgtagctcaatgttgttgttgggtgtgttcatgcttgctcctgaagaatggtgttgatggttttgtaaatgttgtacaggtcggcttcgatggtttgtagctgtttgatttggtggtcgagatcaatgtctgggttgagggtgttgatgcgggaggcgatgtcggtggctgtgcgtagtgtgccgccggtgtggtgaatgatgtgtgccgtgtcggcgagtccggtggtgacggcgtagtgggagaggagaggcatagctggggggtgctccttggcgggttactgttgcgggttgatgttgaggtcggtgacgtgcgggtggtcttctgttccggtgacgaggcagtggacggtgacgggtagtttggatgctccgggctgtttcatggttgcgccgtagacgatggagaaggtgtctttgccaataattttgtggagttggaggtcgatgtcggggttgccgttccatttgacaccttgcgtggcggccttctgttcggctttgcggttgcaggtgtgtgcggcggtgatcatggtgagaccttgtgaggtttcttcaccccttgcctgggcttgcttgtgggttttggcctgctcggctcgcagtgactgttctgcggctgcctgccgtgccgctttctcggctttgcgctgttgggtggttttgggggtccattcggtgttggctgtggtggcctgtggggctggctgtgaggcgagtggcggattgtcgtctggggctggcatgaatgaggcggcggcgatgatggcggctgtgattccggcgatggtgtagcctgttttcttgttcatgactgttgtcccctttccggggtgttgttcgttgctgacatgatcaatcatggtgtgggcggttccccatgtcaaggctgcgctcaacgattgtgagcgatccttgtgtggctaggggtgatggcttctttcgcccaataggatgtgccaccgctggtccagtatccgagtttgttgcgctgcatgcccttcgcctccatctcgtccacggtgaggcacctgcggcgattggggcctgtcttgaccccgtggtcgcctacccggtgcatgtcgcctgaggtggtactcgtgaatgtctcgtgacagactgtgcagtgctctggcttgtatccgatgattgtgctatcgcacttgtggcatgtccattgcatgattgctcctattttccattataagacttcctgtagtgccattttagcgccttgcgagtcttgggggtacaactatataggtcaggtatttctaggcgattctaggctcattgtgtgcgactgggtgttatcgggcacacagggtgagcaggcttccgatgttgatgcggctcacattccagtagagttgtgtggcttcaccgccggtgagtggcttccactcgtcatggctgaacacggtgccatcggatgcgatgaatgtgttggggcgtagcttgtggagttcagtctctacgctctgccggtaggcttcggcgaggccctcaaaatccatgtggtcgcaggagaggttttcgaggcgtgtcaggtcgaaaggctcagggcagtccggggtgggggtgtaaagctgggtgaagtggttggcgatcttctgcatgacgggttccttttctcgtgtggtgggttgatagttttatcgtgtggcttcggcaatgatggcgtccacatcgatcatgtcgatgagatcgttgagttcctcggcctcgttctcggagaggtggcgccagtcgggtggcccgtatacggcaccgtcgagggtgacagtccacaggggccggatgagccgtacggcttcctcgactttggcacggtataggcggcagatgatagacgtgtgggtgttgcctatgtcacatcctgccaggtgtgcggggtggagtgggtggatttctgtctgcccgtagaggctggtgaaggatggtgtgatgagtgtgccatccatgagggtgtgctcctttcggtggtgtatgggttgttgtggtttctagagtgtgtagatggcgatcccatagtcaaggctgcgctcatttggattgagcgtttcatgggatgtggcaggggatgtggcgtatctcacttaagcctttatggcctctctcagcgcctgaaatcttctggcggtaggattatgcagggttgaccctgctaatcgattctaggggccttctagagcgtctcaggggtatgtctgggtgatagcgggtgtggcagatgatctagcgagtcaaggtgccgagctgagacataagatctatcatctaggtgtgtgagatgcatcacatcctcctggcgtggtgtacacccttaaggctactcggtcgatctggcgtggagggtgtagcccagaaatgccgtttaaagctgtgactcggtggattagagcgccttacagggtgggggctaggtatttatacccccaagcaattctgatcgattctagacgcctacaggagcccgatacacgatcagctatccagacgcagatcaccagtccctatcctggttagctcagcctacactatgtggacagtgtggaatactgtaggggaagaaggacacggtaaaagaaagagggaggcatcagccttcaagccttaaggtcttagcgcttagcaccgatggtcttagcagttagcaccgagcccctcaagggctcggcatcagcccgaacaggcacagccctgaaaggagtacacgccatcagggaaggcttgagagtacgaggagccctagcgacgagtactcgaaagcctgagggaacaccctcagcactgatgggcctagcgtgttcggaaaggacacaagagtgaagtgtgagagctgttcgggagtgaaacctgttctgactaggggtttcggtcttaaccaccctcaaaggttacaagactctaagaaaatttaaggaaaagtttaggtttaatttttggacctttactaccaaaaacacccgtttacacccctcaaacccgcctatagagccaaaaccaccagtttgactcatcccaggtggcatatgataggctggacaggtagccagctggacgcaaggccgaaatccgctgacgcggctttcacccttacatccatcagtctaccaaacactttaaagcttcaaggcttagcgctaagcccttaagaccttaacgcttagcaccgagcccctcaagggctcggcatcagtcttaaagctttaaacactttaagtaaacttaagagcttagcacttaaagttaattaataaccttaaaggcttagcacttaaggatataaacttaacatcagtgtttaagactttaaaacttaaaataactattaagacttaaagacttataagctttaaacacttaaagtaactataagactttaaagaccttaagtacttaaagttaaccatcagtcttaaactttaatattataacctataagtcttaaagcttataggtataataatataatataagttataaaagttttagaagagctaaggggttaacttctttacttctctactctctttggttctttctctcttctcttcttttcttcatcaggggagaagaggaacctttaccatcaacgctgatgggctttcaccgtgtgactcgtgtgcttctggtcgcacgctcccggttgcacactcaccacacttactctacctgtgtccctttcaggcttagcgtgttcggctgaaggcgtacggcgtgtcacgctaacacccttaacaccaggtaagacttaaagtgtatattataagtagaagactttaaaaccttaaggtgttcccgcttagcctgtgtcctttagcgctaggcgctaggcgctaagctgtgaaacgcgaacacccatccacccccttttttcttccgtgtccttcttcttttgacaccgctggggggcgatgtgatctttctcacactcatggggggtagtggagaaaacaaccaccccggcacaaacagaacaccccctcaaacgaacaaaacagcccctaggatcgatcagcagggcaagggcaaggtattcatacccccagacgattcccaggccgttacaggagcaatgagaggctcacaggggcaaggggagatcaggggacgtgatggcacataccaaccgcaccgcatccgcctcacaccggcgctggcggcaacgactcatcacccaagcccaacaacaaggccaaaccgaatgcccactctgcggagcaaccatcacctggggcacacacgatctaccaactagccccgaagccgaccacatcacacccgtcagcaggggaggactcaacaccctcgacaacgggcaaatcatctgcagaacatgcaacagaagcaaaggcaatcgcagcgaaccaaacatcaagttccaacaacaaaccacaaaaacattgattccatggtgaaaaacctgccaacccccaccggggacaccccctgcacacccgtgcaagacc

SEQ ID NO: 3 - DNA, 858 bp, Artificial Sequence
  Polynucleotide Encoding Endolysin of C. acnes-Specific Bacteriophage Phi50S
  misc_feature (1)..(1): T, A or G
  misc_feature (856)..(858): TGA, TAA or TAG
dtgagatacattccagcggcgcatcactcggccggatcaaataagccggtgaaccgtgttgtgattcacgcgacatgcccggatgtggggtttccgtccgcttcccgtaaggggcgggcggtgtctacagcaaactattttgcttccccatcgtcgggtggttctgcccattatgtgtgtgatgttggggagacggtgcagtgcttgtcggagtctacgattggttggcatgccccgccgaatccgcattctttgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtrr

SEQ ID NO: 4 - protein, 285 aa, Artificial Sequence
  Edolysin Protein Encoded by SEQ ID NO3
  MISC_FEATURE (1)..(1): Val, Met or Leu
XRYIPAAHHSAGSNKPVNRVVIHATCPDVGFPSASRKGRAVSTANYFASPSSGGSAHYVCDVGETVQCLSESTIGWHAPPNPHSLGIEICADGGSHASFRVPGHAYTREQWLDPRVWPAVERAAILCRQLCDKHGVPKRKLSVSDLKAGKRGVCGHADVTDAWHQSDHDDPGPWFPWDRFMAVVCGGSGESEELTVADVKALHDQIKQLSAQLSGSVNKLHHDVGVVQVQNGDLGKRVDALSWVKNPVTGKLWRTKDALWSVWYYVLECRSRLDRLESAVNGLKK

SEQ ID NO: 5 - DNA, 606 bp, Artificial Sequence
  Polynucleotide Encoding Endolysin of C. acnes-specific Bacteriophage Phi50S
  misc_feature (1)..(1): T, A or G
  misc_feature (604)..(606): TAA, TAG or TGA
dtgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagdrr

SEQ ID NO: 6 - protein, 201 aa, Artificial Sequence
  Endolysin Protein Encoded by SEQ ID NO5
  MISC_FEATURE (1)..(1): Leu, Met or Val
XGIEICADGGSHASFRVPGHAYTREQWLDPRVWPAVERAAILCRQLCDKHGVPKRKLSVSDLKAGKRGVCGHADVTDAWHQSDHDDPGPWFPWDRFMAVVCGGSGESEELTVADVKALHDQIKQLSAQLSGSVNKLHHDVGVVQVQNGDLGKRVDALSWVKNPVTGKLWRTKDALWSVWYYVLECRSRLDRLESAVNGLKK

SEQ ID NO: 7 - DNA, 858 bp, Artificial Sequence; identical to SEQ ID NO: 3
  Polynucleotide Encoding Endolysin of C. acnes-Specific Bacteriophage Phi50M
  misc_feature (1)..(1): T, A or G
  misc_feature (856)..(858): TGA, TAA or TAG

SEQ ID NO: 8 - DNA, 606 bp, Artificial Sequence
  Polynucleotide Encoding Endolysin of C. acnes-Specific Bacteriophage Phi50M
  misc_feature (1)..(1): T, A or G
  misc_feature (604)..(606): TGA, TAA or TAG
dtgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtrr

SEQ ID NO: 9 - protein, 132 aa, Artificial Sequence
  C. acnes-specific bacteriophages Phi50M and Phi50S Holin Protein
  MISC_FEATURE (1)..(1): Leu, Met or Val
XGKQFWLGLLERAAKTFVQTFVAVLGVTAGVTYTAESFRGLPWESALITATVAAVLSVATSFGSPSFVAGKPGKPQLDAGLVPPDDPGIVEPHSVDVSDPGMIEPMDEADVAGYVPKRAAESEVGTVESTVA

SEQ ID NO: 10 - DNA, 399 bp, Artificial Sequence
  Polyncleotide Encoding C. acnes-specific bacteriophages Phi50M and Phi50S Holin Protein
  misc_feature (1)..(1): T, A or G
  misc_feature (397)..(399): TAA, TAG or TGA
dtgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcatrr

SEQ ID NO: 11 - DNA, 22 bp, Artificial Sequence
  Primer 50S_1F
accgtatcatcccacaagtaaa

SEQ ID NO: 12 - DNA, 20 bp, Artificial Sequence
  Primer 50S_1R
gaaacgactccgccgtatag

SEQ ID NO: 13 - DNA, 20 bp, Artificial Sequence
  Primer 50S_2F
gggtggcgtgttgtgagata

SEQ ID NO: 14 - DNA, 20 bp, Artificial Sequence
  Primer 50S_2R
ctgttatcagggccgactcc

SEQ ID NO: 15 - DNA, 20 bp, Artificial Sequence
  Primer 50S_2F-NcA
gggtggcgtaccatgggata

SEQ ID NO: 16 - DNA, 24 bp, Artificial Sequence
  Primer 50S_2F-NcB
gggtggcgtaccatgggatacatt

SEQ ID NO: 17 - DNA, 26 bp, Artificial Sequence
  Primer 50S_3R
aacaaaccaccatcactttttcaaac

SEQ ID NO: 18 - DNA, 26 bp, Artificial Sequence
  Primer 50S_3R-St
aacaaaccaccatccctttttcaaac

SEQ ID NO: 19 - DNA, 22 bp, Artificial Sequence
  Primer UniF
ggtggcctgtgagggtgtgaaa

SEQ ID NO: 20 - DNA, 1470 bp, Cutibacterium acnes (C. acnes) Bacteriophage Phi50S
  misc_feature (1)..(1470): Nucleotide Residues 14,978-16,447 of SEQ ID NO1
gcgggttcggtggcctgtgagggtgtgaaaccatcaccggtggttaccgtatcatcccacaagtaaaagaggaagtgtgttactagtgttgatagtagtgtttgggggtggcgtgttgtgagatacattccagcggcgcatcactcggccggatcaaataagccggtgaaccgtgttgtgattcacgcgacatgcccggatgtggggtttccgtccgcttcccgtaaggggcgggcggtgtctacagcaaactattttgcttccccatcgtcgggtggttctgcccattatgtgtgtgatgttggggagacggtgcagtgcttgtcggagtctacgattggttggcatgccccgccgaatccgcattctttgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtgatggtggtttgttgtgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcataagtgaatatagatgtgtgccccagcggtgctgccacgatcgtgtggtggttgccgctggggcactatttttgtgtctatagtatt

SEQ ID NO: 21 - DNA, 1470 bp, Artificial Sequence; identical to SEQ ID NO: 20
  Variant of Polynucleotide Residues 14,978-16,447 of SEQ ID NO1

SEQ ID NO: 22 - DNA, 20 bp, Artificial Sequence
  Primer 50S_H1F
ccgtcttgacaggcttgagt

SEQ ID NO: 23 - DNA, 20 bp, Artificial Sequence
  Primer 50S_H1R
caaaaatagtgccccagcgg

SEQ ID NO: 24 - DNA, 20 bp, Artificial Sequence
  Primer 50S_H2F
tggtggtttgttgtgggtaa

SEQ ID NO: 25 - DNA, 20 bp, Artificial Sequence
  Primer 50S_H2F-NcA
tggtggtttaccatgggtaa

SEQ ID NO: 26 - DNA, 26 bp, Artificial Sequence
  Primer 50S_H2R
tctatattcacttatgcaacagtaga

SEQ ID NO: 27 - DNA, 26 bp, Artificial Sequence
  Primer 50S_H2R-St
tctatattcacttctgcaacagtaga

SEQ ID NO: 28 - DNA, 630 bp, Cutibacterium acnes (C. acnes) Bacteriophage Phi50S
  misc_feature (1)..(630): Residues 15,818-16,447 of SEQ ID NO1
tcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtgatggtggtttgttgtgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcataagtgaatatagatgtgtgccccagcggtgctgccacgatcgtgtggtggttgccgctggggcactatttttgtgtctatagtatt

SEQ ID NO: 29 - DNA, 630 bp, Artificial Sequence
  Variant of Polynucleotide Residues 15,818-16,447 of SEQ ID NO1
tcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtgatggtggtttgttatgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcagaagtgaatatagatgtgtgccccagcggtgctgccacgatcgtgtggtggttgccgctggggcactatttttgtgtctatagtatt

SEQ ID NO: 30 - DNA, 975 bp, Artificial Sequence
  Polynucleotide for Expressing Endolysin of C. acnes-specific bacteriophages Phi50M and Phi50S
gcgggttcggtggcctgtgagggtgtgaaaccatcaccggtggttaccgtatcatcccacaagtaaaagaggaagtgtgttactagtgttgatagtagtgtttgggggtggcgtgttatgagatacattccagcggcgcatcactcggccggatcaaataagccggtgaaccgtgttgtgattcacgcgacatgcccggatgtggggtttccgtccgcttcccgtaaggggcgggcggtgtctacagcaaactattttgcttccccatcgtcgggtggttctgcccattatgtgtgtgatgttggggagacggtgcagtgcttgtcggagtctacgattggttggcatgccccgccgaatccgcattctttgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagtga

SEQ ID NO: 31 - DNA, 12 bp, Artificial Sequence
  Linker Polynucleotide
tggtggtttgtt

SEQ ID NO: 32 - DNA, 1386 bp, Artificial Sequence
  Polynucleotide Encoding the Endolysin and Holin Protein of C. acnes-specific bacteriophage Phi50S and Phi50M
gcgggttcggtggcctgtgagggtgtgaaaccatcaccggtggttaccgtatcatcccacaagtaaaagaggaagtgtgttactagtgttgatagtagtgtttgggggtggcgtgttatgagatacattccagcggcgcatcactcggccggatcaaataagccggtgaaccgtgttgtgattcacgcgacatgcccggatgtggggtttccgtccgcttcccgtaaggggcgggcggtgtctacagcaaactattttgcttccccatcgtcgggtggttctgcccattatgtgtgtgatgttggggagacggtgcagtgcttgtcggagtctacgattggttggcatgccccgccgaatccgcattctttgggtatagagatttgcgcggatgggggttcgcacgcctcgttccgtgtgccagggcatgcttacacgagggagcagtggctggatcctcgggtgtggcccgccgtggagcgtgccgccatcctgtgtcggcagttgtgtgacaagcatggtgttccgaaaaggaagcttagcgtatccgatttgaaggccggtaaacgtggtgtttgcgggcatgcggatgttacggatgcgtggcatcagtcggatcatgacgatccggggccgtggtttccgtgggacaggtttatggccgtagtctgcggcggtagtggagagagtgaggagttaactgtggctgatgtgaaagccttgcatgatcagattaaacaattgtctgctcagcttagtggttcggtgaataagctgcaccatgatgttggtgtggttcaggtacagaatggtgatttgggtaagcgtgttgacgccctgtcgtgggtgaagaatccggtgacggggaagctgtggcgcaccaaggatgctttgtggagtgtctggtattacgtgttggagtgtcgtagccgtcttgacaggcttgagtctgctgttaacggtttgaaaaagggatggtggtttgttatgggtaaacagttttggttaggtttactggagcgggcggctaagacttttgtgcaaacgtttgttgctgtgttgggggtgacggcgggtgtcacctatacggcggagtcgtttcgcggtttgccgtgggagtcggccctgataacagctacggtggctgcggtgttgtcggtggctacatcgtttggtagcccgtcgtttgtggccggcaagcccggcaagcctcagctggatgcgggtttggttccaccggatgatcccggaatagtggagcctcattcggtggatgtgtcggatcctggcatgatcgagccgatggatgaggctgatgttgccggctatgtgccgaagcgtgccgccgagtcggaggttggcacggtagagtctactgttgcataa

SEQ ID NO: 33 - DNA, 84 bp, Cutibacterium acnes (C. acnes) Bacteriophage Phi50S
gtgaatatagatgtgtgccccagcggtgctgccacgatcgtgtggtggttgccgctggggcactatttttgtgtctatagtatt